SEQUENCE LISTING (12 sequences)

SEQ ID NO: 1 - protein, 1669 aa, Homo sapiens
MGPRLSVWLLLLPAALLLHEEHSRAAAKGGCAGSGCGKCDCHGVKGQKGERGLPGLQGVIGFPGMQGPEGPQGPPGQKGDTGEPGLPGTKGTRGPPGASGYPGNPGLPGIPGQDGPPGPPGIPGCNGTKGERGPLGPPGLPGFAGNPGPPGLPGMKGDPGEILGHVPGMLLKGERGFPGIPGTPGPPGLPGLQGPVGPPGFTGPPGPPGPPGPPGEKGQMGLSFQGPKGDKGDQGVSGPPGVPGQAQVQEKGDFATKGEKGQKGEPGFQGMPGVGEKGEPGKPGPRGKPGKDGDKGEKGSPGFPGEPGYPGLIGRQGPQGEKGEAGPPGPPGIVIGTGPLGEKGERGYPGTPGPRGEPGPKGFPGLPGQPGPPGLPVPGQAGAPGFPGERGEKGDRGFPGTSLPGPSGRDGLPGPPGSPGPPGQPGYTNGIVECQPGPPGDQGPPGIPGQPGFIGEIGEKGQKGESCLICDIDGYRGPPGPQGPPGEIGFPGQPGAKGDRGLPGRDGVAGVPGPQGTPGLIGQPGAKGEPGEFYFDLRLKGDKGDPGFPGQPGMPGRAGSPGRDGHPGLPGPKGSPGSVGLKGERGPPGGVGFPGSRGDTGPPGPPGYGPAGPIGDKGQAGFPGGPGSPGLPGPKGEPGKIVPLPGPPGAEGLPGSPGFPGPQGDRGFPGTPGRPGLPGEKGAVGQPGIGFPGPPGPKGVDGLPGDMGPPGTPGRPGFNGLPGNPGVQGQKGEPGVGLPGLKGLPGLPGIPGTPGEKGSIGVPGVPGEHGAIGPPGLQGIRGEPGPPGLPGSVGSPGVPGIGPPGARGPPGGQGPPGLSGPPGIKGEKGFPGFPGLDMPGPKGDKGAQGLPGITGQSGLPGLPGQQGAPGIPGFPGSKGEMGVMGTPGQPGSPGPVGAPGLPGEKGDHGFPGSSGPRGDPGLKGDKGDVGLPGKPGSMDKVDMGSMKGQKGDQGEKGQIGPIGEKGSRGDPGTPGVPGKDGQAGQPGQPGPKGDPGISGTPGAPGLPGPKGSVGGMGLPGTPGEKGVPGIPGPQGSPGLPGDKGAKGEKGQAGPPGIGIPGLRGEKGDQGIAGFPGSPGEKGEKGSIGIPGMPGSPGLKGSPGSVGYPGSPGLPGEKGDKGLPGLDGIPGVKGEAGLPGTPGPTGPAGQKGEPGSDGIPGSAGEKGEPGLPGRGFPGFPGAKGDKGSKGEVGFPGLAGSPGIPGSKGEQGFMGPPGPQGQPGLPGSPGHATEGPKGDRGPQGQPGLPGLPGPMGPPGLPGIDGVKGDKGNPGWPGAPGVPGPKGDPGFQGMPGIGGSPGITGSKGDMGPPGVPGFQGPKGLPGLQGIKGDQGDQGVPGAKGLPGPPGPPGPYDIIKGEPGLPGPEGPPGLKGLQGLPGPKGQQGVTGLVGIPGPPGIPGFDGAPGQKGEMGPAGPTGPRGFPGPPGPDGLPGSMGPPGTPSVDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 2 - protein, 1712 aa, Homo sapiens
MGRDQRAVAGPALRRWLLLGTVTVGFLAQSVLAGVKKFDVPCGGRDCSGGCQCYPEKGGRGQPGPVGPQGYNGPPGLQGFPGLQGRKGDKGERGAPGVTGPKGDVGARGVSGFPGADGIPGHPGQGGPRGRPGYDGCNGTQGDSGPQGPPGSEGFTGPPGPQGPKGQKGEPYALPKEERDRYRGEPGEPGLVGFQGPPGRPGHVGQMGPVGAPGRPGPPGPPGPKGQQGNRGLGFYGVKGEKGDVGQPGPNGIPSDTLHPIIAPTGVTFHPDQYKGEKGSEGEPGIRGISLKGEEGIMGFPGLRGYPGLSGEKGSPGQKGSRGLDGYQGPDGPRGPKGEAGDPGPPGLPAYSPHPSLAKGARGDPGFPGAQGEPGSQGEPGDPGLPGPPGLSIGDGDQRRGLPGEMGPKGFIGDPGIPALYGGPPGPDGKRGPPGPPGLPGPPGPDGFLFGLKGAKGRAGFPGLPGSPGARGPKGWKGDAGECRCTEGDEAIKGLPGLPGPKGFAGINGEPGRKGDRGDPGQHGLPGFPGLKGVPGNIGAPGPKGAKGDSRTITTKGERGQPGVPGVPGMKGDDGSPGRDGLDGFPGLPGPPGDGIKGPPGDPGYPGIPGTKGTPGEMGPPGLGLPGLKGQRGFPGDAGLPGPPGFLGPPGPAGTPGQIDCDTDVKRAVGGDRQEAIQPGCIGGPKGLPGLPGPPGPTGAKGLRGIPGFAGADGGPGPRGLPGDAGREGFPGPPGFIGPRGSKGAVGLPGPDGSPGPIGLPGPDGPPGERGLPGEVLGAQPGPRGDAGVPGQPGLKGLPGDRGPPGFRGSQGMPGMPGLKGQPGLPGPSGQPGLYGPPGLHGFPGAPGQEGPLGLPGIPGREGLPGDRGDPGDTGAPGPVGMKGLSGDRGDAGFTGEQGHPGSPGFKGIDGMPGTPGLKGDRGSPGMDGFQGMPGLKGRPGFPGSKGEAGFFGIPGLKGLAGEPGFKGSRGDPGPPGPPPVILPGMKDIKGEKGDEGPMGLKGYLGAKGIQGMPGIPGLSGIPGLPGRPGHIKGVKGDIGVPGIPGLPGFPGVAGPPGITGFPGFIGSRGDKGAPGRAGLYGEIGATGDFGDIGDTINLPGRPGLKGERGTTGIPGLKGFFGEKGTEGDIGFPGITGVTGVQGPPGLKGQTGFPGLTGPPGSQGELGRIGLPGGKGDDGWPGAPGLPGFPGLRGIRGLHGLPGTKGFPGSPGSDIHGDPGFPGPPGERGDPGEANTLPGPVGVPGQKGDQGAPGERGPPGSPGLQGFPGITPPSNISGAPGDKGAPGIFGLKGYRGPPGPPGSAALPGSKGDTGNPGAPGTPGTKGWAGDSGPQGRPGVFGLPGEKGPRGEQGFMGNTGPTGAVGDRGPKGPKGDPGFPGAPGTVGAPGIAGIPQKIAVQPGTVGPQGRRGPPGAPGEMGPQGPPGEPGFRGAPGKAGPQGRGGVSAVPGFRGDEGPIGHQGPIGQEGAPGRPGSPGLPGMPGRSVSIGYLLVKHSQTDQEPMCPVGMNKLWSGYSLLYFEGQEKAHNQDLGLAGSCLARFSTMPFLYCNPGDVCYYASRNDKSYWLSTTAPLPMMPVAEDEIKPYISRCSVCEAPAIAIAVHSQDVSIPHCPAGWRSLWIGYSFLMHTAAGDEGGGQSLVSPGSCLEDFRATPFIECNGGRGTCHYYANKYSFWLTTIPEQSFQGSPSADTLKAGLIRTHISRCQVCMKNL

SEQ ID NO: 3 - protein, 1670 aa, Homo sapiens
MSARTAPRPQVLLLPLLLVLLAAAPAASKGCVCKDKGQCFCDGAKGEKGEKGFPGPPGSPGQKGFTGPEGLPGPQGPKGFPGLPGLTGSKGVRGISGLPGFSGSPGLPGTPGNTGPYGLVGVPGCSGSKGEQGFPGLPGTPGYPGIPGAAGLKGQKGAPAKGEDIELDAKGDPGLPGAPGPQGLPGPPGFPGPVGPPGPPGFFGFPGAMGPRGPKGHMGERVIGHKGERGVKGLTGPPGPPGTVIVTLTGPDNRTDLKGEKGDKGAMGEPGPPGPSGLPGESYGSEKGAPGDPGLQGKPGKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEKGETLQPEGQVGVPGDPGLRGQPGRKGLDGIPGTLGVKGLPGPKGELALSGEKGDQGPPGDPGSPGSPGPAGPAGPPGYGPQGEPGLQGTQGVPGAPGPPGEAGPRGELSVSTPVPGPPGPPGPPGHPGPQGPPGIPGSLGKCGDPGLPGPDGEPGIPGIGFPGPPGPKGDQGFPGTKGSLGCPGKMGEPGLPGKPGLPGAKGEPAVAMPGGPGTPGFPGERGNSGEHGEIGLPGLPGLPGTPGNEGLDGPRGDPGQPGPPGEQGPPGRCIEGPRGAQGLPGLNGLKGQQGRRGKTGPKGDPGIPGLDRSGFPGETGSPGIPGHQGEMGPLGQRGYPGNPGILGPPGEDGVIGMMGFPGAIGPPGPPGNPGTPGQRGSPGIPGVKGQRGTPGAKGEQGDKGNPGPSEISHVIGDKGEPGLKGFAGNPGEKGNRGVPGMPGLKGLKGLPGPAGPPGPRGDLGSTGNPGEPGLRGIPGSMGNMGMPGSKGKRGTLGFPGRAGRPGLPGIHGLQGDKGEPGYSEGTRPGPPGPTGDPGLPGDMGKKGEMGQPGPPGHLGPAGPEGAPGSPGSPGLPGKPGPHGDLGFKGIKGLLGPPGIRGPPGLPGFPGSPGPMGIRGDQGRDGIPGPAGEKGETGLLRAPPGPRGNPGAQGAKGDRGAPGFPGLPGRKGAMGDAGPRGPTGIEGFPGPPGLPGAIIPGQTGNRGPPGSRGSPGAPGPPGPPGSHVIGIKGDKGSMGHPGPKGPPGTAGDMGPPGRLGAPGTPGLPGPRGDPGFQGFPGVKGEKGNPGFLGSIGPPGPIGPKGPPGVRGDPGTLKIISLPGSPGPPGTPGEPGMQGEPGPPGPPGNLGPCGPRGKPGKDGKPGTPGPAGEKGNKGSKGEPGPAGSDGLPGLKGKRGDSGSPATWTTRGFVFTRHSQTTAIPSCPEGTVPLYSGFSFLFVQGNQRAHGQDLGTLGSCLQRFTTMPFLFCNVNDVCNFASRNDYSYWLSTPALMPMNMAPITGRALEPYISRCTVCEGPAIAIAVHSQTTDIPPCPHGWISLWKGFSFIMFTSAGSEGTGQALASPGSCLEEFRASPFLECHGRGTCNYYSNSYSFWLASLNPERMFRKPIPSTVKAGELEKIISRCQVCMKKRH

SEQ ID NO: 4 - protein, 1690 aa, Homo sapiens
MWSLHIVLMRCSFRLTKSLATGPWSLILILFSVQYVYGSGKKYIGPCGGRDCSVCHCVPEKGSRGPPGPPGPQGPIGPLGAPGPIGLSGEKGMRGDRGPPGAAGDKGDKGPTGVPGFPGLDGIPGHPGPPGPRGKPGMSGHNGSRGDPGFPGGRGALGPGGPLGHPGEKGEKGNSVFILGAVKGIQGDRGDPGLPGLPGSWGAGGPAGPTGYPGEPGLVGPPGQPGRPGLKGNPGVGVKGQMGDPGEVGQQGSPGPTLLVEPPDFCLYKGEKGIKGIPGMVGLPGPPGRKGESGIGAKGEKGIPGFPGPRGDPGSYGSPGFPGLKGELGLVGDPGLFGLIGPKGDPGNRGHPGPPGVLVTPPLPLKGPPGDPGFPGRYGETGDVGPPGPPGLLGRPGEACAGMIGPPGPQGFPGLPGLPGEAGIPGRPDSAPGKPGKPGSPGLPGAPGLQGLPGSSVIYCSVGNPGPQGIKGKVGPPGGRGPKGEKGNEGLCACEPGPMGPPGPPGLPGRQGSKGDLGLPGWLGTKGDPGPPGAEGPPGLPGKHGASGPPGNKGAKGDMVVSRVKGHKGERGPDGPPGFPGQPGSHGRDGHAGEKGDPGPPGDHEDATPGGKGFPGPLGPPGKAGPVGPPGLGFPGPPGERGHPGVPGHPGVRGPDGLKGQKGDTISCNVTYPGRHGPPGFDGPPGPKGFPGPQGAPGLSGSDGHKGRPGTPGTAEIPGPPGFRGDMGDPGFGGEKGSSPVGPPGPPGSPGVNGQKGIPGDPAFGHLGPPGKRGLSGVPGIKGPRGDPGCPGAEGPAGIPGFLGLKGPKGREGHAGFPGVPGPPGHSCERGAPGIPGQPGLPGYPGSPGAPGGKGQPGDVGPPGPAGMKGLPGLPGRPGAHGPPGLPGIPGPFGDDGLPGPPGPKGPRGLPGFPGFPGERGKPGAEGCPGAKGEPGEKGMSGLPGDRGLRGAKGAIGPPGDEGEMAIISQKGTPGEPGPPGDDGFPGERGDKGTPGMQGRRGELGRYGPPGFHRGEPGEKGQPGPPGPPGPPGSTGLRGFIGFPGLPGDQGEPGSPGPPGFSGIDGARGPKGNKGDPASHFGPPGPKGEPGSPGCPGHFGASGEQGLPGIQGPRGSPGRPGPPGSSGPPGCPGDHGMPGLRGQPGEMGDPGPRGLQGDPGIPGPPGIKGPSGSPGLNGLHGLKGQKGTKGASGLHDVGPPGPVGIPGLKGERGDPGSPGISPPGPRGKKGPPGPPGSSGPPGPAGATGRAPKDIPDPGPPGDQGPPGPDGPRGAPGPPGLPGSVDLLRGEPGDCGLPGPPGPPGPPGPPGYKGFPGCDGKDGQKGPMGFPGPQGPHGFPGPPGEKGLPGPPGRKGPTGLPGPRGEPGPPADVDDCPRIPGLPGAPGMRGPEGAMGLPGMRGPPGPGCKGEPGLDGRRGVDGVPGSPGPPGRKGDTGEDGYPGGPGPPGPIGDPGPKGFGPGYLGGFLLVLHSQTDQEPTCPLGMPRLWTGYSLLYLEGQEKAHNQDLGLAGSCLPVFSTLPFAYCNIHQVCHYAQRNDRSYWLASAAPLPMMPLSEEAIRPYVSRCAVCEAPAQAVAVHSQDQSIPPCPQTWRSLWIGYSFLMHTGAGDQGGGQALMSPGSCLEDFRAAPFLECQGRQGTCHFFANKYSFWLTTVKADLQFSSAPAPDTLKESQAQRQKISRCQVCVKYS

SEQ ID NO: 5 - protein, 1685 aa, Homo sapiens
MKLRGVSLAAGLFLLALSLWGQPAEAAACYGCSPGSKCDCSGIKGEKGERGFPGLEGHPGLPGFPGPEGPPGPRGQKGDDGIPGPPGPKGIRGPPGLPGFPGTPGLPGMPGHDGAPGPQGIPGCNGTKGERGFPGSPGFPGLQGPPGPPGIPGMKGEPGSIIMSSLPGPKGNPGYPGPPGIQGLPGPTGIPGPIGPPGPPGLMGPPGPPGLPGPKGNMGLNFQGPKGEKGEQGLQGPPGPPGQISEQKRPIDVEFQKGDQGLPGDRGPPGPPGIRGPPGPPGGEKGEKGEQGEPGKRGKPGKDGENGQPGIPGLPGDPGYPGEPGRDGEKGQKGDTGPPGPPGLVIPRPGTGITIGEKGNIGLPGLPGEKGERGFPGIQGPPGLPGPPGAAVMGPPGPPGFPGERGQKGDEGPPGISIPGPPGLDGQPGAPGLPGPPGPAGPHIPPSDEICEPGPPGPPGSPGDKGLQGEQGVKGDKGDTCFNCIGTGISGPPGQPGLPGLPGPPGSLGFPGQKGEKGQAGATGPKGLPGIPGAPGAPGFPGSKGEPGDILTFPGMKGDKGELGSPGAPGLPGLPGTPGQDGLPGLPGPKGEPGGITFKGERGPPGNPGLPGLPGNIGPMGPPGFGPPGPVGEKGIQGVAGNPGQPGIPGPKGDPGQTITQPGKPGLPGNPGRDGDVGLPGDPGLPGQPGLPGIPGSKGEPGIPGIGLPGPPGPKGFPGIPGPPGAPGTPGRIGLEGPPGPPGFPGPKGEPGFALPGPPGPPGLPGFKGALGPKGDRGFPGPPGPPGRTGLDGLPGPKGDVGPNGQPGPMGPPGLPGIGVQGPPGPPGIPGPIGQPGLHGIPGEKGDPGPPGLDVPGPPGERGSPGIPGAPGPIGPPGSPGLPGKAGASGFPGTKGEMGMMGPPGPPGPLGIPGRSGVPGLKGDDGLQGQPGLPGPTGEKGSKGEPGLPGPPGPMDPNLLGSKGEKGEPGLPGIPGVSGPKGYQGLPGDPGQPGLSGQPGLPGPPGPKGNPGLPGQPGLIGPPGLKGTIGDMGFPGPQGVEGPPGPSGVPGQPGSPGLPGQKGDKGDPGISSIGLPGLPGPKGEPGLPGYPGNPGIKGSVGDPGLPGLPGTPGAKGQPGLPGFPGTPGPPGPKGISGPPGNPGLPGEPGPVGGGGHPGQPGPPGEKGKPGQDGIPGPAGQKGEPGQPGFGNPGPPGLPGLSGQKGDGGLPGIPGNPGLPGPKGEPGFHGFPGVQGPPGPPGSPGPALEGPKGNPGPQGPPGRPGLPGPEGPPGLPGNGGIKGEKGNPGQPGLPGLPGLKGDQGPPGLQGNPGRPGLNGMKGDPGLPGVPGFPGMKGPSGVPGSAGPEGEPGLIGPPGPPGLPGPSGQSIIIKGDAGPPGIPGQPGLKGLPGPQGPQGLPGPTGPPGDPGRNGLPGFDGAGGRKGDPGLPGQPGTRGLDGPPGPDGLQGPPGPPGTSSVAHGFLITRHSQTTDAPQCPQGTLQVYEGFSLLYVQGNKRAHGQDLGTAGSCLRRFSTMPFMFCNINNVCNFASRNDYSYWLSTPEPMPMSMQPLKGQSIQPFISRCAVCEAPAVVIAVHSQTIQIPHCPQGWDSLWIGYSFMMHTSAGAEGSGQALASPGSCLEEFRSAPFIECHGRGTCNYYANSYSFWLATVDVSDMFSKPQSETLKAGDLRTRISRCQVCMKRT

SEQ ID NO: 6 - protein, 1691 aa, Homo sapiens
MLINKLWLLLVTLCLTEELAAAGEKSYGKPCGGQDCSGSCQCFPEKGARGRPGPIGIQGPTGPQGFTGSTGLSGLKGERGFPGLLGPYGPKGDKGPMGVPGFLGINGIPGHPGQPGPRGPPGLDGCNGTQGAVGFPGPDGYPGLLGPPGLPGQKGSKGDPVLAPGSFKGMKGDPGLPGLDGITGPQGAPGFPGAVGPAGPPGLQGPPGPPGPLGPDGNMGLGFQGEKGVKGDVGLPGPAGPPPSTGELEFMGFPKGKKGSKGEPGPKGFPGLRGPPGFPGLGTTGEKGEKGEKGIPGLPGPRGPMGSEGVQGPPGQQGKKGTLGFPGLNGFQGIEGQKGDIGLPGPDVFIDIDGAVISGNPGDPGDPGLPGLKGDEGIQGLRGPSGVPGLPALSGVPGALGPQGFPGLKGDQGNPGRTTIGAAGLPGRDGLPGPPGPPGPPSPEFETETLHNKEAGFPGLRGEQGPKGNLGLKGIKGDSGFCACDGGVPNTGPPGEPGPPGPWGLIGLPGLKGARGDQGSGGAQGPAGAPGLVGPLGPSGPKGKKGEPILSTIQGMPGDRGDSGSQGFRGVIGEPGKDGVPGLPGLPGLPGDGGQGFPGEKGLPGLPGEKGHPGPPGLPGNGLPGLPGPRGLPGDKGKDGLPGQQGLPGSKGITLPCIIPGSYGPSGFPGTPGFPGPKGSRGLPGTPGQPGSSGSKGEPGSPGLVHLPELPGFPGPRGEKGLPGFPGLPGKDGLPGMIGSPGLPGSKGATGDIFGAENGAPGEQGLQGLTGHKGFLGDSGLPGLKGVHGKPGLLGPKGERGSPGTPGQVGQPGTPGSSGPYGIKGKSGLPGAPGFPGISGHPGKKGTRGKKGPPGSIVKKGLPGLKGLPGNPGLVGLKGSPGSPGVAGLPALSGPKGEKGSVGFVGFPGIPGLPGISGTRGLKGIPGSTGKMGPSGRAGTPGEKGDRGNPGPVGIPSPRRPMSNLWLKGDKGSQGSAGSNGFPGPRGDKGEAGRPGPPGLPGAPGLPGIIKGVSGKPGPPGFMGIRGLPGLKGSSGITGFPGMPGESGSQGIRGSPGLPGASGLPGLKGDNGQTVEISGSPGPKGQPGESGFKGTKGRDGLIGNIGFPGKKGEDGKVGVSGDVGLPGAPGFPGVAGMRGEPGLPGSSGHQGAIGPLGSPGLIGPKGFPGFPGLHGLNGLPGTKGTHGTPGPSITGVPGPAGLPGPKGEKGYPGIGIGAPGKPGLRGQKGDRGFPGLQGPAGLPGAPGISLPSLIAGQPGDPGRPGLDGERGRPGPAGPPGPPGPSSNQGDTGDPGFPGIPGPKGPKGDQGIPGFSGLPGELGLKGMRGEPGFMGTPGKVGPPGDPGFPGMKGKAGPRGSSGLQGHPGQTPTAEAVQVPPGPLGLPGIDGIPGLTGDPGAQGPVGLQGSKGLPGIPGKDGPSGLPGPPGALGDPGLPGLQGPPGFEGAPGQQGPFGMPGMPGQSMRVGYTLVKHSQSEQVPPCPIGMSQLWVGYSLLFVEGQEKAHNQDLGFAGSCLPRFSTMPFIYCNINEVCHYARRNDKSYWLSTTAPIPMMPVSQTQIPQYISRCSVCEAPSQAIAVHSQDITIPQCPLGWRSLWIGYSFLMHTAAGAEGGGQSLVSPGSCLEDFRATPFIECSGARGTCHYFANKYSFWLTTVEERQQFGELPVSETLKAGQLHTRVSRCQVCMKSL

SEQ ID NO: 7 - protein, 225 aa, Homo sapiens
GFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 8 - protein, 224 aa, Homo sapiens
GYLLVKHSQTDQEPMCPVGMNKLWSGYSLLYFEGQEKAHNQDLGLAGSCLARFSTMPFLYCNPGDVCYYASRNDKSYWLSTTAPLPMMPVAEDEIKPYISRCSVCEAPAIAIAVHSQDVSIPHCPAGWRSLWIGYSFLMHTAAGDEGGGQSLVSPGSCLEDFRATPFIECNGGRGTCHYYANKYSFWLTTIPEQSFQGSPSADTLKAGLIRTHISRCQVCMKNL

SEQ ID NO: 9 - protein, 225 aa, Homo sapiens
GFVFTRHSQTTAIPSCPEGTVPLYSGFSFLFVQGNQRAHGQDLGTLGSCLQRFTTMPFLFCNVNDVCNFASRNDYSYWLSTPALMPMNMAPITGRALEPYISRCTVCEGPAIAIAVHSQTTDIPPCPHGWISLWKGFSFIMFTSAGSEGTGQALASPGSCLEEFRASPFLECHGRGTCNYYSNSYSFWLASLNPERMFRKPIPSTVKAGELEKIISRCQVCMKKR

SEQ ID NO: 10 - protein, 226 aa, Homo sapiens
GFLLVLHSQTDQEPTCPLGMPRLWTGYSLLYLEGQEKAHNQDLGLAGSCLPVFSTLPFAYCNIHQVCHYAQRNDRSYWLASAAPLPMMPLSEEAIRPYVSRCAVCEAPAQAVAVHSQDQSIPPCPQTWRSLWIGYSFLMHTGAGDQGGGQALMSPGSCLEDFRAAPFLECQGRQGTCHFFANKYSFWLTTVKADLQFSSAPAPDTLKESQAQRQKISRCQVCVKYS

SEQ ID NO: 11 - protein, 225 aa, Homo sapiens
GFLITRHSQTTDAPQCPQGTLQVYEGFSLLYVQGNKRAHGQDLGTAGSCLRRFSTMPFMFCNINNVCNFASRNDYSYWLSTPEPMPMSMQPLKGQSIQPFISRCAVCEAPAVVIAVHSQTIQIPHCPQGWDSLWIGYSFMMHTSAGAEGSGQALASPGSCLEEFRSAPFIECHGRGTCNYYANSYSFWLATVDVSDMFSKPQSETLKAGDLRTRISRCQVCMKRT

SEQ ID NO: 12 - protein, 225 aa, Homo sapiens
GYTLVKHSQSEQVPPCPIGMSQLWVGYSLLFVEGQEKAHNQDLGFAGSCLPRFSTMPFIYCNINEVCHYARRNDKSYWLSTTAPIPMMPVSQTQIPQYISRCSVCEAPSQAIAVHSQDITIPQCPLGWRSLWIGYSFLMHTAAGAEGGGQSLVSPGSCLEDFRATPFIECSGARGTCHYFANKYSFWLTTVEERQQFGELPVSETLKAGQLHTRVSRCQVCMKSL

(19) United States
(12) Patent Application Publication
(10) Pub. No.: US 2018/0207240 A1
(43) Pub. Date: Jul. 26, 2018

(54) COLLAGEN IV REPLACEMENT

(71) Applicant: Goldfinch Biopharma, Inc., Cambridge, MA (US)

(72) Inventors: Bradley L. Hodges, Milford, MA (US); Thomas M. Barnes, Brookline, MA (US); Philip R. Reilly, Concord, MA (US); Walter E. Kowtoniuk, South Boston, MA (US)

(21) Appl. No.: 15/328,215

(22) PCT Filed: Jul. 23, 2015
(86) PCT No.: PCT/US15/41712

§ 371 Date: Jan. 23, 2017

Related U.S. Application Data
(60) Provisional application No. 62/128,729, filed on Mar. 5, 2015.
(60) Provisional application No. 62/072,490, filed on Oct. 30, 2014.
(60) Provisional application No. 62/029,135, filed on Jul. 25, 2014.

(51) Int. Cl. A61K 38/39; A61P 13/12; A61K 45/06; C07K 14/78

(52) U.S. Cl. CPC A61K 38/39; C07K 14/78; A61K 45/06; A61P 13/12

(57) ABSTRACT

Disclosed are pharmaceutical compositions, formulations, and methods for treating Alport syndrome by administering recombinant human collagen IV protein to a patient in need.

### CROSS REFERENCES

This application claims priority of U.S. Provisional Application Ser. No. 62/128,729 filed on Mar. 5, 2015; U.S. Provisional Application Ser. No. 62/072,490 filed on Oct. 30, 2014; and U.S. Provisional Application Ser. No. 62/029,135, filed on Jul. 25, 2014; the content of each of which is herein incorporated by reference in their entirety.

### REFERENCE TO THE SEQUENCE LISTING

The present application is being filed along with a Sequence Listing in electronic format. The Sequence Listing is provided as a file entitled 20721004PCTSEQLST.txt, created on Jul. 23, 2015, which is 100,507 bytes in size. The information in the electronic format of the sequence listing is incorporated herein by reference in its entirety.

### FIELD OF THE INVENTION

The present invention relates to collagen replacement for treating collagen associated diseases, in particular collagen IV and Alport syndrome. Provided are recombinant collagen IV molecules, pharmaceutical compositions and methods for treating collagen IV associated disorders such as Alport syndrome.

### BACKGROUND OF THE INVENTION

Alport Syndrome is an inherited disease that primarily affects the glomeruli, the tiny tufts of capillaries in the kidneys that filter wastes from the blood. The earliest symptom of the disease is blood in the urine (hematuria). Patients often present hearing loss and/or ocular complications as well. Fifty percent of Alport patients develop end stage renal disease (ESRD) by age 20 with a median time of death of 25 years of age and ninety percent by age 45. Without intervention progression to ESRD is inexorable. Alport syndrome has been reported worldwide without restriction to particular geographic areas. The prevalence is estimated to be about 1 in 5000 newborns in the United States. In UK, about 40 per million (including disease carriers) persons suffer Alport syndrome and Alport patients account for about 1% of patients on renal transplantation therapy. The incidence of Alport syndrome was found to be 1:53,000 in Finland and 1:17,000 in southern Sweden (Pajari et al., Acta Paediatr, 1996, 85, 1300-1306; and Persson et al., Clin Nephrol, 2005, 64, 85-90).

The glomerular basement membrane (GBM) is the site of the Alport lesion. Characteristic GBM ultrastructure changes in patients with Alport syndrome are irregular thickening of the GBM and multilamellation of the lamina densa forming a “basket weave” pattern. These changes are minimal in the early stages of the disease, but are widespread in adult patients. The widespread changes of the GBM are indicative of a tendency towards a progressive disease course. A good correlation between the severity of the GBM irregular thickening and the clinical course has been reported (Basta-Jovanovic et al., Am J Kid Dis, 1990, 16, 51-56). Young patients are likely the most amenable to therapy.

Alport syndrome is caused by changes in genes (mutations) that affect type IV collagen, a protein that is important to the normal structure and function of glomerular basement membrane. This disease is mainly due to recessive mutations in the Collagen IV genes (COL4A3, COL4A4 or COL4A5) that encode collagen IV α3, α4 and α5 chains. Since COL4A5 is X-linked, a single defective gene in males is sufficient to produce the disease. Collagen IV α3-α4-α5 is an important constituent of glomerular basement membranes in the kidney.

Diagnosis of Alport Syndrome relies on careful evaluation of the patient's signs and symptoms, along with their family history. Sometimes hearing and vision tested. The evaluation can also include blood tests, urine tests, and a kidney biopsy to determine Alport syndrome. A genetic test can help confirm the diagnosis and determine the genetic type of Alport syndrome.

Currently, aside from renal transplant, ACE inhibitors are the only therapy, and these can delay ESRD. Alport patients impose a heavy burden on the health care system, comprising 1-2% of all European ESRD patients and 2-3% of all US patients requiring renal transplant. Furthermore, transplantation often leads to immune rejection of the transplanted allografts. Therefore, there is an unmet medical need to develop novel therapies for this serious and life threatening rare disorder.

Medical researchers are very interested in understanding why people with Alport syndrome develop kidney failure, and in developing treatments that can slow or prevent the development of kidney failure. Several treatments are being tested in animals with a condition equivalent to Alport syndrome, including inhibitors of enzymes which mediate collagen IV assembly and stem cell therapy. Given the fact that collagen IV protein is the key component of the GBM and is deficient in Alport GBM, the present invention develops a novel treatment for Alport syndrome in which functional recombinant human collagen IV (rhCol4) protein is delivered back to the affected GBM. It is shown, according to the present invention, surprisingly that a recombinant human collagen would easily exit the vasculature and embed in the affected GBM. Such collagen IV replacement could restore the filtering function of the glomeruli in the kidney, therefore treat Alport syndrome.

### SUMMARY OF THE INVENTION

The present invention relates to collagen replacement for treating collagen associated diseases, in particular collagen IV and Alport syndrome. Provided are recombinant collagen IV proteins, pharmaceutical compositions and methods for treating collagen IV associated disorders such as Alport syndrome.

In some embodiments, the invention provides pharmaceutical compositions and formulations that include recombinant collagen IV protein and one or more pharmaceutically acceptable excipients which facilitate collagen IV stability, delivery, penetration and/or functionality. The recombinant collagen IV protein can be collagen IV protomers, dimers, tetramers, multimers and/or the mixture thereof. The collagen IV protomer may contain three polypeptides selected from the group consisting of α1(IV), α2(IV), α3(IV), α4(IV), α5(IV) and α6(IV) chain polypeptides.

In some embodiments, the collagen IV protomer is a heterotrimer comprising an α3(IV) chain polypeptide, an α4(IV) chain polypeptide and an α5(IV) chain polypeptide, wherein the α3(IV) chain polypeptide comprises the amino acid sequence of SEQ ID NO. 3 and variants thereof; the α4(IV) chain polypeptide comprises the amino acid sequence of SEQ ID NO. 4 and variants thereof; and the α5(IV) chain polypeptide comprises the amino acid sequence of SEQ ID NO.5 and variants thereof.

In other embodiments, the collagen IV protomer is a heterotrimer comprising two copies of α1(IV) chain polypeptides, and an α2(IV) chain polypeptide, wherein the α1(IV) chain polypeptide comprises the amino acid sequence of SEQ ID NO. 1 and variants thereof; the α2(IV) chain polypeptide comprises the amino acid sequence of SEQ ID NO. 2 and variants thereof.

Because specific T cell epitopes that can drive immune rejection are found in the NC1 domain of the α(IV) chains, in other embodiments, said collagen IV protomer is a heterotrimer comprising one, two or three chimeric collagen IV α polypeptides selected from the chimeric α3(IV), α4(VI) and α5(IV) polypeptides. As disclosed in the present invention, the chimeric α3(IV) chain polypeptide is a chimeric polypeptide in which all or part of the NC1 domain of the α3(IV) chain is replaced with all or part of the NC1 domain of the α1(IV) and/or α2(IV) chains. The chimeric α4(IV) chain polypeptide is a chimeric polypeptide in which all or part of the NC1 domain of the α4(IV) chain is replaced with all or part of the NC1 domain of the α1(IV) and/or α2(IV) chains. The chimeric α5 (IV) chain polypeptide is a chimeric polypeptide in which all or part of the NC1 domain of the α5 (IV) chain is replaced with all or part of the NC1 domain of the α1(IV) and/or α2(IV) chains.

As an example of the recombinant collagen IV protomer containing chimeric α(IV) polypeptides, a collagen IV heterotrimeric protomer may consist of one chimeric α3(IV) chain polypeptide in which all or part of the NC1 domain of the α3(IV) chain is replaced with all or part of the NC1 domain of α1(IV) or α2(IV) chains; one chimeric α4(IV) chain polypeptide in which all or part of the NC1 domain of the α4(IV) chain is replaced with all or part of the NC1 domain of α1(IV) or α2(IV) chains; and one chimeric α5(IV) chain polypeptide in which all or part of the NC1 domain of the α5(IV) chain is replaced with all or part of the NC1 domain of α1(IV) or α2(IV) chains.

In some aspects, the NC1 domains of α1(IV), α2(IV), α3(IV), α4(IV), α5(IV) comprise the amino acid sequences of SEQ ID NO.7, SEQ ID NO.8, SEQ ID NO.9, SEQ ID NO.10 and SEQ ID NO.11, respectively.

In other embodiments, said recombinant collagen IV protein is in the form of collagen IV dimers, which comprise two protomers that are dimerized non-covalently or covalently, wherein the protomers may be the heterotrimer α3(IV)-α4(IV)-α5(IV), or the heterotrimer comprising chimeric α3(IV), α4(IV) and/or α5(IV) chains.

In some embodiments, said recombinant collagen IV is recombinant human collagen IV, in particular human collagen IV α3-α4-α5.

According to the present invention, collagen IV protein may be produced via the extraction and purification of human natural collagen IV from collagen IV containing tissues and organs, or through expression of recombinant collagen IV protein in mammalian cell lines, insects, plant cells and/or bacteria and yeast. In some aspects, the collagen IV protein is further modified to achieve a particular percentage of 3-hydroxyproline, 4-hydroxyproline and/or hydroxylysine, as compared to naturally occurring collagen IV protein. For example, the collagen IV protein of the present invention contains about 6.5% to about 14% of 4-hydroxyprolines (i.e. between 65-140 3-hydroxyproline residues/1000 AA) and/or about 0.2% to about 1.6% of 3-hydroxyprolines (i.e. between 6-16 3-hydroxyproline residues/1000 AA).

In a further aspect, as tested in the present invention, the collagen IV protein used in the present invention may contain modified amino acids and/or other amino acid substitutes. Such modifications and substitutes would not change the functionality of collagen IV protein, but may improve some chemical and physical features of collagen IV protein, such as increased stability, and reduced immunoreactivity.

In one embodiment, the pharmaceutical composition comprising recombinant human collagen IV protein may be used for improving glomerular structures and functions in a patient with Alport syndrome, wherein the recombinant human collagen IV protein comprises collagen IV protein protomers, dimers, tetramers, multimers and/or the mixture thereof, and one or more pharmaceutically acceptable excipients, wherein said collagen IV protein protomers, dimers, multimers consisting of three α chain polypeptides selected from the group consisting of α3 (IV), α 4 (IV) and α5 (IV) chain polypeptides.

According to the present invention, the pharmaceutically acceptable excipients comprise one or more antioxidants, one or more tonicity agents, one or more chelators, and agents that can assist in collagen IV assembly in the glomerular sites, such as bromine.

Provided in the present inventions also include methods, vectors, chimeric cDNA constructs, cell lines and functional assays for producing normal and chimeric collagen IV α polypeptides of the present invention. In some aspects, the host cells may be genetically engineered to express prolyl 3-hydroxylase and/or prolyl 4-hydroxylase. In other aspects, the host cells may be further deficient in peroxidasin, lysyl oxidase, and/or native collagen IV protein or collagens other than native collagen IV.

In some embodiments, the present invention features methods for treating a condition characterized by one or more deficiencies of collagen IV protein in a subject in need thereof by administering to the subject in need thereof a pharmaceutical composition comprising recombinant collagen IV protein. Said condition could be characterized by one or more deficiencies of the α3(IV) chain polypeptide; one or more deficiencies of the α4(IV) chain polypeptide; and/or one or more deficiencies of the α5(IV) chain polypeptide. In particular, such deficiencies are due to genetic mutations in COL4A3, COL4A4 and/or COL4A5 genes.

In some aspects, the condition characterized by deficiencies of collagen IV protein is selected from Alport syndrome, thin basement membrane nephropathy (TBMN), familial hematuria, end stage renal disease (ESRD), progressive renal insufficiency, glomerular hematuria, proteinuria, hereditary nephritis, diabetic nephropathy, perinatal cerebral hemorrhage and porencephaly, hemorrhagic stroke, and any diseases or disorder with defects in collagen IV protein.

In a preferred embodiment, the disease is Alport syndrome. Alport syndrome may be X-linked Alport syndrome, autosomal recessive Alport syndrome, or autosomal dominant Alport syndrome. An X-linked Alport syndrome may be caused by any mutation in the COL4A5 gene encoding the α5(IV) chain polypeptide. An autosomal recessive Alport syndrome may be caused by any mutations in COL4A3 and/or COL4A4 genes encoding the α4(IV) chain polypeptide and α5(IV) chain polypeptide, respectively. An autosomal dominant Alport syndrome may be caused by any mutations in COL4A3 and/or COL4A4 genes encoding the α4(IV) chain polypeptide and α5(IV) chain polypeptide, respectively.

In other aspects, the patient with Alport syndrome may be a patient without renal dysfunction findings who is diagnosed by family history or by genetic testing.

In some embodiments, the pharmaceutical compositions used in the present methods comprising recombinant collagen IV protomers, dimers, tetramers, multimers and the mixture thereof. In some aspects, the recombinant collagen IV consists of protomers. Collagen IV protomers are heterotrimers consisting of one α3(IV) chain, one α4(IV) chain and one α5(IV) chain, wherein the three chains form a triple helix and wherein the α3(IV) chain comprises the amino acid sequence of SEQ ID NO.3; the α4(IV) chain comprises the amino acid sequence of SEQ ID NO.4 and the α5(IV) chain comprises the amino acid sequence of SEQ ID NO.5.

In other aspects, the recombinant collagen IV protomers are heterotrimers comprising one, two or three chimeric α(IV) chains selected from the chimeric α3(IV), α4(IV), α5(IV) chains, wherein the chimeric α3(IV) chain comprises a chimeric polypeptide in which all or part of the NC1 domain of the α3(IV) chain is replaced with all or part of the NC1 domain of the α1(IV) or α2(IV) chains; the chimeric α4(IV) chain comprises a chimeric polypeptide in which all or part of the NC1 domain of the α4(IV) chain is replaced with all or part of the NC1 domain of the α1(IV) or α2(IV) chains; and the chimeric α5(IV) chain comprises a chimeric polypeptide in which all or part of the NC1 domain of the α5(IV) chain is replaced with all or part of the NC1 domain of the α1(IV) or α2(IV) chains.

In other embodiments, said recombinant collagen IV are in the form of collagen IV dimers, wherein said dimers comprise two collagen IV protomers which may be recombinant collagen IV α3-α4-α5 and/or chimeric collagen IV as disclosed herein. In some aspects, said collagen IV dimers are dimerized enzymatically or chemically in vitro prior to administering to the subject in need.

In some embodiments, the collagen IV protein is administered to a subject in need thereof by an intravenous injection, intraperitoneal injection, intramuscular injection, subcutaneous injection, intrathecal injection, intracerebral ventricular administration, intracranial delivery, intraocular delivery, intraaural delivery, and/or by an acute or chronically placed catheter. In a preferred embodiment, the collagen IV protein is administered to a subject in need thereof by intravenous injection.

In some embodiments, the effective dose is between about 100 ng/kg and about 100 mg/kg. In some aspects, the effective dose is between about 100 ng/kg and about 100 μg/kg. In other aspects, the effective dose is between about 1 μg/kg to about 1 mg/kg. In further other aspects, the effective dose is between about 1 mg/kg and about 100 mg/kg. In one embodiment, the effective dose is about 5 mg/kg.

One or more prophylactic drugs may be co-administered with the collagen IV protein composition to a subject in need, said prophylactic drugs may be anti-thrombotic agents and/or anti-inflammatory drugs.

Anti-thrombotic agents may be used to primarily prevent, or secondarily prevent acute thrombus formation induced by recombinant collagen IV replacement. An anti-thrombotic agent may be an antiplatelet drug, an anticoagulant, or a thrombolytic drug. Antiplatelet drugs may include, but are not limited to, irreversible cyclooxygenase inhibitors such as aspirin and triflusal; adenosine diphosphate (ADP) receptor inhibitors such as clopidogrel, prasugrel, ticagrelor and ticlopidine; phosphodiesterase inhibitors such as cilostazol; glycoprotein IIB/IIIA inhibitors such as abciximab, eptifibatide and tirofiban; adenosine reuptake inhibitors such as dipyridamole; thromboxane inhibitors such as thromboxane synthase inhibitors, thromboxane receptor antagonists and teruthroban. Anticoagulants may include, but are not limited to, warfarin, heparin, acenocoumarol, atromentin, brodifacoum and phenindione. Thrombolytic drugs may include, but are not limited to, tissue plasminogen activator t-PA such as alteplase, reteplase and tenecteplase; anistreplase; streptokinase and urokinase.

Anti-inflammatory agents may include, but are not limited to, NSAIDS (non-steroidal anti-inflammatory drugs) such as aspirin, ibuprofen, naproxen; acetaminophen; and ImSAIDs (immune-selective anti-inflammatory drugs).

In some embodiments, the present invention features methods for preventing, ameliorating, reversing, slowing, halting and/or improving one or more abnormalities comprising thinning and splitting glomerular basement membrane (GBM), heavy proteinuria, mild proteinuria, hematuria, renal deficiency, progression to end stage renal disease, auditory dysfunction, ocular abnormalities, porencephaly, brain small vessel disease with hemorrhage, brain small vessel disease with Axenfeld-Rieger anomaly, hereditary angiopathy with nephropathy, aneurysms, and muscle, and/or intracerebral hemorrhage, by administering to a subject in need thereof a pharmaceutical composition that comprises recombinant collagen IV protein, such that administering collagen IV protein prevents, ameliorates, slows, halts and/or improves the phenotypic outcomes of the subject.

The collagen IV protein may be administered to a mammal. The mammal may be a mouse, a rat, a dog or a human.

In addition, assays that may be used to detect recombinant collagen IV in basement membranes are provided in the present invention. Said assays may include receptor binding, cell migration, differentiation and/or adhesion, and biomarker measurement.

### BRIEF DESCRIPTION OF THE DRAWINGS

FIG. 1 is a representative denaturing/non-reducing SDS-PAGE gel image of Col4 (α1₍₂₎α2) protein which is immune blotted with anti-Col4 antibodies: sc70246 (1:100) (Lanes 4-7), ab6586 (1:1000) (Lanes 8-11) and ab19808 (1:1000) (Lanes 12-15). Lanes 1 and 2 are molecular weight markers from Novex. For each antibody, different amounts of Col4 (α1₍₂₎α2) protein (250 ng, 125 ng, 25 ng, 12.5 ng) were loaded. The bands: individual α(IV) chains (I), protomers (P), dimers (D) and tetramers (T) were visualized with HRP conjugated anti-IgG secondary antibodies (1:20,000 dilution).

FIG. 2 shows Col4 (α1₍₂₎α2) species in denaturing SDS-PAGE (4-15% gel) with or without disulfide reduction. FIG. 2a is a representative denaturing SDS-PAGE gel image of Col4 (α1₍₂₎α2) preparation without disulfide reduction. FIG. 2b a representative denaturing SDS-PAGE gel image of Col4 (α1₍₂₎α2) preparation with disulfide reduction. Lanes 13, 14 and 15 of FIGS. 2a and 2b are fully reduced LAM-111 and only the gamma1 chain of LAM-111 is assayed by a gamma1 specific antibody (Cat. No. sc5584).

FIG. 3 is a representative native PAGE gel image of Col4 (α1₍₂₎α2) proteins with charge shift using Direct Red 80 dye. LAM-111 was used as an independent molecular weight marker.

FIG. 4 is a histogram of ELISA assay for FITC-Col4 (α1₍₂₎α2) conjugate detection using various anti-FITC antibodies.

FIG. 5a is a representative gel image that shows the detection of FITC labeled and unlabeled Col4 (α1₍₂₎α2). Col4 (α1₍₂₎α2) is reduced in lanes A-C and unreduced in lanes D-F. The same amount of protein was loaded in each lane. Lanes A and D were loaded with unlabeled Col4 (α1₍₂₎α2); Lanes B and E were loaded with FITC labeled Col4 (α1₍₂₎α2) but unpurified by a size exclusion column and Lanes C and F were loaded with FITC labeled Col4 (α1₍₂₎α2) and purified by a size exclusion column.

FIG. 5b is a representative gel image of immunoblot using anti-FITC antibody (ab19492, 1:20,000 dilution) for detection of FITC-Col4 (α1₍₂₎α2).

FIG. 6a is a histogram of ELISA assay for FITC-LAM-111 conjugate detection using various anti-FITC antibodies.

FIG. 6b is a representative gel image that shows the detection of FITC labeled and unlabeled LAM-111. LAM-111 is reduced in lanes A-B and unreduced in lanes D-F. The same amount of protein was loaded in each lane. Lanes A and D were loaded with unlabeled LAM-111; Lanes B and E were loaded with FITC labeled LAM-111 but unpurified by a size exclusion column and Lanes C and F were loaded with FITC labeled LAM-111 and purified by a size exclusion column.

FIG. 6c is a representative gel image of immunoblot using anti-FITC antibody (ab19492, 1:20,000 dilution) for detection of FITC-LAM-111.

FIG. 7 shows the localization of FITC-Col4 (α1₍₂₎α2) and FITC-LAM-111 in the glomerular basement membrane (GBM) after 6 doses of intravenous injection. FIGS. 7a and 7b are representative confocal fluorescence microscopy images of kidneys of Heterozygous (Col4+/− (hybrid)) mouse that is un-injected (FIG. 7a) and Alport (Col4−/− (Hybrid)) mouse that is injected with 6 doses of FITC-Col4 (α1₍₂₎α2) (FIG. 7b) and. The top panel are images of anti-FITC antibody staining; the middle ones are images of anti-agrin staining and the bottom panel are overlap images of anti-FITC and anti-agrin staining with a DNA marker DAPI staining. FIGS. 7c and 7d are representative confocal fluorescence microscopy images of kidneys of Heterozygous (Col4+/− (B6)) mouse that is un-injected (FIG. 7c) and Alport (Col4−/− (B6)) mouse that is injected with 6 doses of FITC-LAM-111 (FIG. 7d). The top panel are images of anti-FITC antibody staining; the middle ones are images of anti-agrin staining and the bottom panel are overlap images of anti-FITC and anti-agrin staining with a DNA marker DAPI staining.

FIG. 8a shows representative images of glomerular morphology in un-injected Alport mouse (Col4−/−75 days old). FIG. 8b shows representative images of glomerular morphology in Col4−(α1₍₂₎α2) dosed Alport mouse (Col4−/−, 88 days old).

FIG. 9 is a histogram of glomerular sclerosis in Alport mice (Col4−/−) either treated with Col4−(α1₍₂₎α2) (N=5), or untreated (N=4), or treated with control vehicle only (N=3). At least 100 glomeruli from each mouse at postnatal day 70 were counted and the percentages indicate the average number of non-sclerotic glomeruli in each cohort. Bars represent range of values in each cohort. The Non-Col4 (N=7) represents the combined data from uninjected and vehicle injected Alport mice.

FIG. 10 shows representative electron microscopy images of glomerular capillaries. FIG. 10a are representative images of heterozygous mouse (Col4+/−) injected with vehicle only (day 70). FIG. 10b are representative images of Alport mouse (Col4−/−) injected with vehicle (day 70). FIG. 10c are representative images of Alport mouse (Col4−/−) injected with Col4−(α1₍₂₎α2) protein (day 70).

FIG. 11 is blood urea nitrogen (BUN) measurement in Col4−(α1₍₂₎α2) dosed Alport mice (upper) and untreated/vehicle treated Alport mice (lower).

FIG. 12 is urine albumin/creatinine ratio of Col4−(α1₍₂₎α2) dosed Alport mice (upper) and untreated/vehicle treated Alport mice (lower).

### DETAILED DESCRIPTION OF THE INVENTION

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

The present invention relates to pharmaceutical compositions, medications and methods for treating collagen associated diseases, in particular, diseases characterized by one or more deficiencies of collagen IV protein, such as Alport syndrome caused by genetic mutations in the COL4A3, COL4A4 and COL4A5 genes that encode collagen IV α3, α4 and α5 chain polypeptides. The present invention aims to transport functional collagen IV protein back to the affected sites to restore collagen IV based structural support and other physiological functions.

Collagen is the major structural constituent of mammals. Numerous diseases and conditions are associated with excess accumulation of collagen in tissue, mutations of collagen α chains, abnormal assembly, increased/decreased post-translational modifications, and/or interrupted collagen interaction with other structural proteins. Mutations in any of collagen α chain polypeptides cause a variety of rare diseases due to the absence of correct collagen structures, which provide support for tissues and organs, present signals for development, and/or support physiological functions. For example, the absence of collagen IV caused by mutations in COL4A3, COL4A4 and COL4A5 genes impairs the glomerular basement membranes, which may ultimately result in renal failure.

It has been an unmet issue how to restore the absent or abnormal collagen for treatment of collagen mediated disorders. The present invention provides novel pharmaceutical compositions, medications and methods for treating collagen mediated disorders, in particular the collagen IV mediated disorder Alport syndrome. Provided here are also methods for treating Alport syndrome, and/or preventing, slowing the process of renal failure.

Mutations in genes that encode collagen IV α3, α4 and α5 chains (COL4A3, COL4A4 and COL4A5) could cause Alport syndrome, which is characterized by glomerulonephritis, end stage kidney disease, hearing loss and ocular dysfunction. Currently there is no specific treatment for Alport Syndrome. The same treatments that are used in people with high blood pressure and other symptoms of kidney disease are used in people with Alport syndrome. Kidney transplantation is usually very successful in people with Alport syndrome, and is considered the best treatment when end-stage kidney failure is approaching. However, many patients develop Alport post-transplant nephritis (APTN) which is an aggressive form of anti-glomerular basement membrane disease.

The rationale of the present invention is to transport recombinant human collagen IV protein back to the affected sites such as glomerular basement membrane to restore its normal structure and therefore its filtering function. Previously, several studies have shown that large proteins can penetrate into glomerular basement membranes. Endothelial fenestrae are about 100-150 nm, large enough to permit the passage of large proteins, such as ferritin, but it is not known whether elongated molecules, such as a collagen IV protomer, or an even more elongated collagen IV dimer, is capable of penetrating into the GBM. Nephrotic glomerular basement membrane is more permeable to ferritin than the normal glomerular basement membrane. Therefore, the present invention develops pharmaceutical compositions and methods for treating Alport syndrome by administering to the affected patient recombinant collagen IV protein, in particular collagen IV protomers, dimers, tetramers or multimers by intravenous injection. We disclose the novel finding that collagen IV protomers, dimers, tetramers or multimers will penetrate into the glomerular basement membrane in the kidney and embed into the extracellular matrix network with other components.

In addition, the pharmaceutical composition comprising recombinant collagen IV may also be used as part of regenerative medications. As a non-limiting example, the recombinant collagen IV from the present invention may be incorporated into artificial scaffolds and/or natural, decellularized scaffolds; mixed with other extracellular matrix proteins; employed as substrates for the in vivo, ex vivo and/or in vitro growth, differentiation and selection of stem cells; or employed as a thrombosis enhancing patch for acute wound pair.

### Definitions

Unless defined otherwise, all technical and scientific terms used herein have the meaning commonly understood by a person skilled in the art to which this invention belongs. The following terms have the meanings ascribed to them unless specified otherwise. And the definitions will be helpful to understand the present invention as set forth herein.

The term “protomer”, as used herein, refers to a molecular structural subunit of a large macromolecule (i.e. oligomeric protein). In the context of collagens, the collagen protomers themselves are trimers, consisting of three α chain polypeptides. For example, a collagen IV protomer is a heterotrimer of three α chain polypeptides. Collagen protomers will form dimers, tetramers, oligomers and multimers.

As used herein, the term “basement membrane”, also referred to as “basal lamina”, means the thin spread of fibrils. Basement membrane is composed of at least several identified proteins and peptide derivatives, including several specific types of collagen (e.g., Type IV and Types I-V), laminin, and various types of cell adhesion molecules (CAMs), proteoglycans, and fibronectin. The basement membrane forms a thin sheet of fibers that underlies cells in various tissues (e.g., skin). Basement membrane primarily serves as the anchoring system of cells, attaching it to the connective tissue below, or provides a protective barrier against foreign objects or malignant cells, or filters blood through the glomerulus in the kidneys.

As used herein, the term “glomerular basement membrane (GBM)” refers to the basement membrane of the glomerulus in the kidneys, serving as extracellular matrix component of the glomerular filtration barrier. It is flanked by the podocyte and glomerular endothelial cell layers. The major GBM components are laminin-521, collagen α3-α4-α5 (IV), nidogen, and the heparan sulfate proteoglycan agrin.

The terms “polypeptide” “peptide” and “protein” are used interchangeably herein to refer to a polymer of amino acid residues, and not to a specific length. Thus, peptides, oligopeptides and protein fragments are included within the definition of polypeptide. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. Another example of post-translation modification is hydroxylation of proline and lysine in many collagen polypeptides. The terms “polypeptide,” “peptide” and “protein” include glycoproteins, as well as non-glycoproteins.

As used herein, the term “treating” or “treatment” refers to administering a pharmaceutical composition, e.g., a composition of the present invention comprising collagen IV protein, for prophylactic and/or therapeutic purpose. To “prevent disease” refers to prophylactic treatment of a patient who is not ill yet, but who is susceptible to, or otherwise at risk of developing a particular disease. For example, a patient, by genetic test, carries mutations in COL4A3, COL4A4 and/or COL4A5 genes. To “treat disease” refers to administering to a patient who is already suffering from a disease to ameliorate the disease and improve the patient's condition, e.g., renal function.

Other features and advantages of the present invention are discussed in the following detailed description and the claims.

### Collagen

Collagen is the most abundant protein found in the mammals, constituting about 25% of total protein. It is the main fibrous component of skin, bone, tendon, cartilage and periodontium. A typical collagen molecule is a long, rod-like, rigid structure with triple stranded helix. Collagen is further cross-linked to form polymeric collagen structure/networks, such as fibrils, sheets and filaments. The collagen superfamily of proteins plays a dominant role in maintaining the integrity of various tissues and also has a number of other important functions.

Although collagen molecules are found throughout the body, their types and organization are dictated by the structural role collagen plays in a particular organ/tissue. In some organs, collagen may be dispersed as a gel that gives support to the structure, as in the extracellular matrix or the vitreous humor of the eye. In other organs, collagen may be bundled in tight, parallel fibers that provide great strength, as in tendons. The collagen fibers of bone may be arranged particularly so as to resist mechanical attack.

### Types of Collagen

Collagen is a large family of highly developed fibrous proteins comprising more than 25 collagen types (see Table 1) that form highly organized super molecular assemblies, as well as additional proteins that have collagen-like domains. Many genetically, chemically and immunologically distinct types of collagens have also been identified. Collagen variations may be due to differences in the assembly of basic polypeptide chains, different lengths of the helix, various interruptions in the helix, difference in the terminations of the helical domains and/or cleavage of the non-collagenous domains.

Collagen can be organized into several groups, based on their locations and functions in the body. Collagen types I, II, III, V and XI are fibril-forming collagens, which form linear polymers of fibrils having characteristic banded patterns, reflecting the regular staggered packing of the individual collagen molecules in the fibrils. Collagen types IX, XII, XIV and XVI are fibril associated collagens that bind to the surface of collagen fibrils, linking these fibrils to one another and/or to other components in the extracellular matrix. Collagen types IV, VIII and X are network forming collagens, which form a three dimensional mesh, rather than fibrils. For example, collagen IV molecules assemble into a sheet that constitutes a major part of basement membranes. A fourth group of collagen includes all other collagens, such as collagen VI (beaded fibril forming collagen) and VII (anchoring fibrils).

### Structural Features of Collagen

All collagen molecules consist of three polypeptides, referred to as α chains, which wind around one another for at least a portion of their length to form a triple α helix. The parts of collagen that do not form triple helices are called non-collagenous, or “NC” domains, and are numbered within each collagen e.g., NC1, NC2 etc. The individual α chain polypeptide has similar domain organization, containing a large central triple helix forming domain with numerous Gly-X—Y repeats (i.e. collagenous domain), flanked by small N- and C-terminal global domains (i.e. non-collagenous domains). Some types of triple helical collagen protomers contain three genetically identical α chains forming homotrimers, whereas others contain two or three different α chains forming heterotrimers.

H Bonds:

The three α chain polypeptides are held together and stabilized by hydrogen bonds between them. Unlike the more common α helix, the collagen helix has no intrachain hydrogen bonds.

Amino Acid Sequences:

The collagen helical domain contains specific amino acids (glycine, proline and hydroxyproline) which are important in the formation of the triple helix. These amino acids have a regular arrangement in each a chain polypeptide. The sequence often follows the pattern Gly-X—Y, where X is frequently proline and Y is often hydroxyproline (it can also be hydroxylysine). Thus, most of the helical part of the α chain can be regarded as a polytripeptide whose sequence can be represented as (-Gly-Pro-Hyp-)ₙ. Proline or hydroxyproline constitute about ⅙ of the total sequence and Glycine accounts for ⅓ of the sequence. Proline facilitates the formation of helical orientation of each a chain because its ring structure causes “kinks” in the peptide chain. Glycine is found in every third position of the triple repeat. Because glycine is the smallest, nonpolar amino acid with no side chain, it plays a unique role in fibrous structural proteins. The side chain of glycine is a hydrogen atom and such a small side chain makes it easy to fit into places where no other amino acids can. For example, only glycine can be in the internal amino acid of a collagen helix.

Collagens do not contain chemically reactive side groups like those in enzymes and transport proteins.

Triple Helical Structure:

Unlike most globular proteins that are folded into compact structures, collagen, a fibrous protein, has an elongated, triple-helical structure that places many of its amino acid side chains on the surface of the triple-helical molecule. Each a chain forms a left-handed helix and they align together to form a triple right-handed helical protomer. The α chains each are shaped into a left handed symmetry because of the high content of proline and hydroxyproline rings, with their geometrically constrained carboxyl and (secondary) amino groups along with abundance of glycine. The left handed helices are formed without any intrachain hydrogen bonding. The triple helix may be continuous stretch or it may be interrupted by non collagenous elements.

Hydroxyproline and Hydroxylysine:

Collagen contains hydroxyproline (Hyp) and hydroxylysine (Hyl), which are not present in most other proteins. These residues result from the post-translational hydroxylation of some of the proline and lysine residues. The hydroxylation reactions are catalyzed by enzymes (hydroxylase) and require ascorbic acid (vitamin C). Hydroxyproline is important in stabilizing the triple-helical structure of collagen because it maximizes interchain hydrogen bond formation.

Glycosylation:

In some cases, the hydroxyl group of the hydroxylysine residues of collagen may be enzymatically glycosylated, making collagen a glycoprotein. Most commonly, glucose and galactose are sequentially attached to the polypeptide chain prior to triple-helix formation.

Cross-Linkage:

The tensile strength of collagen depends on the formation of covalent intermolecular cross-links between the individual protein subunits. The fibril containing collagens in higher vertebrates (e.g., types I, II, III, V and XI) are cross-linked through a mechanism based on the reactions of aldehydes generated enzymatically from lysine (or hydroxylysine) side-chains by lysyl oxidase. Certain other collagen types (e.g. collagen IX of cartilage) are also cross-linked by the lysyl oxidase mechanism.

### Biosynthesis of Collagen

The major sites for the synthesis of the polypeptide precursors of the collagen molecules are mesenchymal cells and their derivatives including fibroblasts, chondrocytes (in cartilage), osteoblasts (in bone), odontoblasts and cementoblasts. Other cells may include, but are not limited to, epithelial cells, endothelial cells, muscle cells and Schwann cells.

The precursor polypeptides are formed inside cells through sequential events including translation of prepro-α chains from specific mRNAs, cleavage of signal peptide (pro-α chain), proline hydroxylation, lysine hydroxylation, hydroxylysine glycosylation and association of C-terminal peptides/disulphide bond formation/incorporation of C terminal propeptides (procollagen molecules). The collagen molecules are then secreted into the extracellular matrix. After enzymatic modification, the mature collagen monomers aggregate and become cross-linked to form collagen fibers.

Formation of Pro α Chains:

Like most proteins produced for export/secretion, the newly synthesized polypeptide precursors of α chains (prepro-α chains) contain a special signal sequence at their N-terminal ends. The signal sequence facilitates the binding of ribosomes to the rough endoplasmic reticulum (RER), and directs the passage of the prepro-α chain into the lumen of the RER. The signal sequence is rapidly cleaved in the RER to yield a precursor of collagen called a pro-α chain.

Post-Translational Modification:

The pro-α chains are processed by a number of enzymes within the lumen of the RER while the polypeptides are still being synthesized. Proline and lysine residues found in the Y-position of the -Gly-X—Y-sequence can be hydroxylated to form hydroxyproline and hydroxylysine residues. These hydroxylation reactions require molecular oxygen, Fe²⁺, and the reducing agent ascorbic acid (vitamin C). Two hydroxylating enzymes, prolyl hydroxylase and lysyl hydroxylase, are usually involved. Lack of prolyl and lysyl hydroxylation can impair interchain H-bond formation, as is formation of a stable triple helix. Additionally, collagen fibrils cannot be cross-linked (see below), greatly decreasing the tensile strength of the assembled fiber. Hydroxyproline may also prevent denaturation of collagen fibers in temperature changes. It has been shown that non hydroxylated triple helices undergo denaturation at temperature below 37° C. Some hydroxylysine residues are modified by glycosylation with glucose or glucosyl-galactose.

Triple Helix Assembly:

After hydroxylation and glycosylation, three pro-α chains form a procollagen molecule (protomer) that has a central collagenous region of triple helix flanked by the nonhelical N- and C-terminal domains called propeptides.

The formation of procollagen molecule begins with a series of noncovalent interactions between the C-terminal non-collagenous domains of the three pro α chains, which provide correct alignment for the nucleation of triple helix formation through the middle collagenous domains. This first recognition of C-terminal propeptides selects specific chains for the procollagen assembly. For example, procollagen types I and III are assembled in a type specific manner despite both being synthesized in skin fibroblasts and having high levels of identity in their procollagen α chain sequences. While collagen I exists as a heterotrimer of two pro α1(I) and one pro α2 (I) chains, collagen III is an obligate homotrimer comprising three pro α1(III) chains.

Secretion:

The procollagen molecules move through the Golgi apparatus, where they are packaged in secretory vesicles. The vesicles fuse with the cell membrane, causing the release of procollagen molecules into the extracellular space.

Sequential biosynthetic events occur in the extracellular space through which procollagen is processed into mature collagen. Such events include N-terminal and C-terminal domain (propeptide) cleavage (by N- and C-proteinase), alignment of collagen molecules that form microfibril (lysine/hydroxylysine terminal NH2 oxidation (Cu²⁺-containing lysyl oxidase)), and final fibril formation (reducible cross-link formation and maturation of cross-links). The fibrils are immature and lack strength. These immature fibrils are cross linked and gradually form mature collagen fibers. Cross-linkage is a slow process and tensile strength of collagen steadily increases over a long period via growth and reorganization of fibers.

Extracellular Cleavage of Propeptides:

For most procollagen molecules, the terminal non-collagenous domains (propeptides) are cleaved off by N- and C-procollagen peptidases, after their release into the extracellular space. The cleaved tropocollagen will cross link one another to form collagen fibers or other structures.

Many of these propeptides have important functions that are distinct from those of the collagen domains. For example, endostatin, a fragment released from collagen type XVIII, potently inhibits angiogenesis and tumor growth.

Formation of Collagen Fibers:

Individual tropocollagen molecules spontaneously associate to form collagen fibrils. They form an ordered, overlapping, parallel array, with adjacent collagen molecules arranged in a staggered pattern, each overlapping its neighbor by a length approximately three-quarters of a molecule. As used herein, the term “tropocollagen” refers to the collagen subunit in which the N-terminal and C-terminal propeptides are cleaved.

Cross-Linkage:

Cross linkage is catalyzed by extracellular enzyme lysyl oxidase. This Cu²⁺-containing extra-cellular enzyme oxidatively deaminates some of lysyl and hydroxylysyl residues in collagen. The reactive aldehydes that result (allysine and hydroxyallysine) can condense with lysyl or hydroxylysyl residues in neighboring collagen molecules to form covalent cross-links and, thus, mature collagen fibers then the reactive aldehydes combine with collagen residues to form cross-links.

### Degradation of Collagen

Normal collagen is highly stable, having a half-life as long as several years. However, breakdown of collagen is a key component of any normal tissue that is undergoing morphogenesis and growth. Connective tissue is dynamic and is constantly being remodeled, for example, in response to injury of tissues. It is vital that this process is kept under tight control. Collagen destruction is mediated primarily by the collagenases, which are part of a large family of matrix metalloproteinase (MMPs). Collagenases are specialized enzymes that have evolved specifically to hydrolyze collagens, because the triple helix structure is resistant to most of common proteinases. For example, the cleavage site of collagen I is specific, generating three-quarter and one-quarter length fragments. These fragments are further degraded by other matrix proteinases to their constituent amino acids.

Collagen biosynthesis is tightly regulated during normal development and homeostasis in a cell and in a tissue specific manner. It has been shown that a variety of growth factors and cytokines regulate collagen production during development, inflammation, wound healing and other physiological conditions (e.g., PDGF, TGF-beta, FGF and IGF, IL-1, IFN-gamma, THF-alpha and glucocorticoids). Some of those post-translational enzymes may be attractive targets for the development of drugs to treat collagen accumulation in many fibrotic diseases.

### Collagen Diseases

As the main component of connective tissue, it is unavoidable that defects in collagen proteins may affect many systems of human body, from the central nervous system to the musculoskeletal and cardiovascular systems. A wide spectrum of diseases is caused by the more than 1000 mutations that thus have been identified in about 22 collagen genes. These mutations include deletions, small insertions, RNA splicing mutations, nonsense mutations, and/or missense mutations. Some examples of collagen diseases include osteogenesis imperfecta, many chondrodysplasias, several subtypes of the Ehlers-Danlos syndrome, Alport syndrome, Bethlem myopathy, certain subtypes of epidermolysis bullosa, Knobloch syndrome and also some cases of osteoporosis, arterial aneurysms, osteoarthrosis, and intervertebral disc disease (See Table 1). The characterization of mutations in additional collagen genes will probably add further diseases to this list.

TABLE 1
Collagen and Diseases

| | | | Proposed | Cells of | |
|---|---|---|---|---|---|
| Type | Gene(s) | Characteristics | function | origins | Disorders |
| I | COL1A1 | The most | Fibril | Fibroblasts; | Osteogenesis |
| | COL1A2 | abundant | forming; | reticular cells; | imperfecta; Ehlers- |
| | | collagen of the | provide | smooth | Danlos Syndrome, |
| | | human body; | tensile | muscle cells | types 1, 2, 7; Infantile |
| | | mostly present | strength | | cortical hyperostosis |
| | | in scar tissue, | | | (Caffey's disease) |
| | | the end product | | | |
| | | when tissue | | | |
| | | heals by repair; | | | |
| | | commonly | | | |
| | | found in | | | |
| | | tendons, skin, | | | |
| | | artery walls, the | | | |
| | | endomysium of | | | |
| | | myofibrils, | | | |
| | | fibrocartilage, | | | |
| | | and the organic | | | |
| | | part of bones | | | |
| | | and teeth. | | | |
| II | COL2A1 | Hyaline | Fibril | | Collagenopathy; |
| | | cartilage; makes | forming; | | Hypochondrogenesis; |
| | | up 50% of all | provide | | Achondrogenesis |
| | | cartilage | tensile | | type 2; Stickler |
| | | protein; vitreous | strength | | syndrome; Marshall |
| | | humor of the | | | syndrome; |
| | | eye; inter- | | | Spondyloepipphyseal |
| | | veterbral disk | | | dysplasia congenita; |
| | | | | | Spondyloepimetaphy |
| | | | | | seal dysplasia, |
| | | | | | strudwick type |
| III | COL3A1 | This is the | Fibril | Fibroblasts; | Ehlers-Danlos |
| | | collagen of | forming; | Endothelial | Syndrome (type IV) |
| | | granulation | fetal skin, | cells; reticular | |
| | | tissue, and is | blood | cells | |
| | | produced | vessels; | | |
| | | quickly by | provide | | |
| | | young | tensile | | |
| | | fibroblasts | strength | | |
| | | before the | | | |
| | | tougher type I | | | |
| | | collagen is | | | |
| | | synthesized. | | | |
| | | Reticular fiber. | | | |
| | | Also found in | | | |
| | | artery walls, | | | |
| | | skin, intestines | | | |
| | | and the uterus. | | | |
| IV | COL4A1 | Basement | Network | Podocytes; | Alport syndrome; |
| | COL4A2 | membrane; eye | forming; | epithelial and | Goodpasture's |
| | COL4A3 | lens. Also serves | interacts | endothelial | syndrome |
| | COL4A4 | as part of the | with | cells | |
| | COL4A5 | filtration system | laminin | | |
| | COL4A6 | in capillaries | and | | |
| | | and the | heparan | | |
| | | glomeruli of | sulfate; | | |
| | | nephron in the | major | | |
| | | kidney. | component | | |
| | | | of | | |
| | | | basement | | |
| | | | membranes | | |
| V | COL5A1 | Most interstitial | Connector | Fibroblasts; | Ehlers-Danlos |
| | COL5A2 | tissue, | between | smooth | Syndrome (types 1 |
| | COL5A3 | associated with | basement | muscle cells. | and 2, Classic) |
| | | collagen I, | membrane | | |
| | | associated with | and stroma, | | |
| | | placenta. | promotes | | |
| | | | cell | | |
| | | | attachment | | |
| | | | and | | |
| | | | migration | | |
| VI | COL6A1 | Most interstitial | Matrix | Fibroblasts | Ullrich congenital |
| | COL6A2 | tissue, associate | assembly; | | muscular dystrophy; |
| | COL6A3 | with type I | attach cells | | Bethlem Myopathy |
| | | collagen. | to | | |
| | | Collagen VI | connective | | |
| | | microfibrils are | tissues | | |
| | | found in a wide | | | |
| | | variety of | | | |
| | | extracellular | | | |
| | | matrices, | | | |
| | | including | | | |
| | | muscle, skin, | | | |
| | | tendon, | | | |
| | | cartilage, | | | |
| | | intervertebral | | | |
| | | discs, lens, | | | |
| | | internal organs | | | |
| | | and blood | | | |
| | | vessels. | | | |
| VII | COL7A1 | Forms | Network | Fibroblasts | Epidermolysis |
| | | anchoring fibrils | forming; | | bullosa dystrophica; |
| | | in dermal | mostly | | recessive dystrophic |
| | | epidermal | beneath | | epidermolysis |
| | | junctions | stratified | | bullosa; Bart |
| | | | squamous | | syndrome; Transient |
| | | | epithelia. | | bullous dermolysis of |
| | | | Links basal | | the newborn |
| | | | surface of | | |
| | | | epithelial | | |
| | | | cells with | | |
| | | | underlying | | |
| | | | connective | | |
| | | | tissue, | | |
| | | | anchoring | | |
| | | | fibers | | |
| VIII | COL8A1 | Some | Stabilization | Corneal | Posterior |
| | COL8A2 | endothelial | of | fibroblasts | polymorphous |
| | | cells; | cellular | | corneal dystrophy 2; |
| | | Descemet's | phenotype | | Fuchs' dystrophy 1 |
| | | membrane; | and | | |
| | | cornea | maintenance | | |
| | | | of | | |
| | | | cellular | | |
| | | | integrity | | |
| IX | COL9A1 | FACIT collagen, | Fibril | | Epiphyseal dysplasia, |
| | COL9A2 | cartilage, | associated. | | Multiple, 2 (EDM2); |
| | COL9A3 | associates with | Attaches to | | EDM 3 and EDM 6 |
| | | type II and XI | type II | | |
| | | fibrils | collagen | | |
| | | | and | | |
| | | | mediates | | |
| | | | binding of | | |
| | | | other | | |
| | | | connective | | |
| | | | tissue | | |
| | | | elements. | | |
| X | COL10A1 | Hypertrophic | Facilitates | | Schmid metaphyseal |
| | | and mineralizing | removal of | | dysplasia |
| | | cartilage | hypertrophic | | |
| | | | cartilage; | | |
| | | | facilitates | | |
| | | | conversion | | |
| | | | of cartilage | | |
| | | | to bone | | |
| XI | COL11A1 | Cartilage | Regulates | | Weissenbacher- |
| | COL11A2 | | the | | Zweymuller |
| | | | diameter of | | syndrome; |
| | | | type II | | otospondylomegaepiphyseal |
| | | | collagen | | dysplasia |
| | | | and | | |
| | | | mediates | | |
| | | | collagen | | |
| | | | protcoglycan | | |
| | | | interactions | | |
| XII | COL12A1 | FACIT collagen, | Fibril | Fibroblasts | Ehlers-Danlos |
| | | interacts with | associated; | | myopathy; similar to |
| | | type I containing | tendon; | | Bethlem myopathy |
| | | fibrils, decorin | ligaments. | | |
| | | and | Attaches to | | |
| | | glycosaminoglycans | type I | | |
| | | | collagen | | |
| | | | and | | |
| | | | mediates | | |
| | | | binding of | | |
| | | | other | | |
| | | | connective | | |
| | | | tissue | | |
| | | | elements | | |
| XIII | COL13A1 | Transmembrane | Plasma | | No known disease |
| | | collagen, | membrane | | |
| | | interacts with | | | |
| | | integrin α1β1, | | | |
| | | fibronectin and | | | |
| | | components of | | | |
| | | basement | | | |
| | | membranes like | | | |
| | | nidogen and | | | |
| | | perlecan. | | | |
| XIV | COL14A1 | FACIT | | Fibroblasts | Palmoplantar |
| | | collagen; | | | keratoderma |
| | | all tissues | | | |
| XV | COL15A1 | | | | No known disease |
| XVI | COL16A1 | | | | Crohn's |
| | | | | | inflammatory bowel |
| | | | | | disease |
| XVII | COL17A1 | Transmembrane | Hemidesmosome | Keratinocytes | Bullous pemphigoid |
| | | collagen, also | | | and certain forms of |
| | | known as | | | epidermolysis bullosa |
| | | BP180, a 180 kDa | | | |
| | | protein | | | |
| XVIII | COL18A1 | Source of | | | Knobloch syndrome |
| | | endostatin; | | | |
| | | plays roles in | | | |
| | | retinal structure | | | |
| | | and in neural | | | |
| | | tube closure. | | | |
| XIX | COL19A1 | FACIT collagen | | | No known disease |
| XX | COL20A1 | | | | No known disease |
| XXI | COL21A1 | FACIT collagen | | | No known disease |
| XXII | COL22A1 | | | | No known disease |
| XXIII | COL23A1 | MACIT | | | Congenital |
| | | collagen | | | Hypertrichosis |
| XXIV | COL24A1 | | | | No known disease |
| XXV | COL25A1 | | | | Antisocial personality |
| | | | | | disorder |
| XXVI | EMID2 | | | | No known disease |
| XXVII | COL27A1 | | | | Steel Syndrome |
| XXVIII | COL28A1 | | | | No known disease |
| XXIX | COL29A1 | Epidermal | | | Atopic dermatis |
| | | collagen | | | |

In addition to diseases of collagen deficiency caused by genetic mutations in genes encoding collagen polypeptides, many autoimmune disorders occur when the immune system affect collagens, such as vascular diseases. Collagen vascular diseases include, but are not limited to, ankylosing spondylitis, dermatomyositis, polyarteritis nodosa, psoriatic arthritis, rheumatoid arthritis, scleroderma and systemic lupus erythematosus.

Furthermore, defects in any one of the many steps in collagen fiber synthesis (e.g., collagen modifying enzyme defects) can result in a genetic disease involving an inability of collagen to form fibers properly and, thus, provide tissues with the needed tensile strength normally provided by collagen.

### Collagen Medical Uses

Collagen is widely used in the medical field. The most common use of collagen is in cosmetic surgery and as wound healing aids in burn patients. Collagen can be used in the construction of artificial skin substitutes used in the management of severe burns. Collagen is widely used as reconstruction of bone, and for a wide variety of dental, orthopedic and surgical purposes. Other uses include wound dressing and as matrices for tissue growth.

Because of the biochemical features of collagen, collagen has been used in many other fields, such as applications in cell culture (for cell attachment, studying cell behavior and cellular interaction with the extracellular environment, etc.); as barrier films/sheets; for drug delivery such as collagen hydrogel, collagen-liposomes, collagen nanoparticles/nanosphere, and collagen tablets/pellets, biodegradable materials and substitutes.

Collagen medical uses are widely discussed in the art, such as collagen sponges for drug delivery (see e.g., U.S. Pat. Nos. 3,157,524; 4,412,947; and 5,512,301); collagen film (see, e.g., U.S. Pat. No. 3,014,024); collagen hydrogel (see, e.g., U.S. Pat Nos. 5,108,424; 5,213,701); collagen as wound healing agents (see, e.g., U.S. Pat. Nos. 3,810,473; 4,841,962; 4,837,285; 4,925,924; 5,081,106; and 5,766,631); making contact lens (see, e.g., U.S. Pat No. 4,268,131); collagen nanoparticles (See, e.g., U.S. Pat. Nos. 5,932,245; and 8,668,926; and U.S. patent publication No. 20130323311); nerve repair (see, e.g., U.S. patent publication No. 20110276066); collagen implants for a variety of purposes such as cartilage repair, prosthetics, orthopedic grafts, tendon replacement implant, implant for soft tissue and bone implant (see, e.g., U.S. Pat. Nos. 3,272,204; 4,424,208; 5,171,273; 5,523,291; 6,080,194; 7,544,212; and 7, 595,062; and U.S. patent publication Nos. 20080305517; 2010108945; and 20110264237); modified collagen for therapeutic and diagnostic uses (see, e.g., U.S. Pat. Nos. 7,183,383 and 8,283,414; and U.S. patent publication No. 20130116405).

### Collagen Production

Most of collagen used for medical purpose is bovine collagen from certified BSE (Bovine spongiform encephalopathy) free cattle. Other commonly used include porcine tissue and equine tissue. In some cases, a human patient's own fat, hyaluronic acid or polyacrylamide gel are also used. Human collagen may be extracted from donor cadavers, placentas and aborted fetuses, which has a low possibility of immune reactions.

Many recombinant techniques have been developed for producing recombinant collagen proteins. Those methods for producing recombinant collagen proteins through bioengineering are well known to skilled in art. Some exemplary methods include production of human recombinant collagen in the milk of transgenic animals (see, e.g., U.S. Pat. Nos. 5,667,839; 5,895,833; 5,962,648; and 6,111,165); production of mammal recombinant collagen in plant cells (see, e.g., U.S. Pat. Nos. 6,617,431; 7,232,886); production of mammal recombinant collagen in mammalian cells, insects, and microorganisms such as bacteria and yeast (see, e.g., U.S. Pat. Nos. 6,150,081; 7,932,353; 8,084,579; 8,188,230; and U.S. patent publication No. 20020142391; 20140107036); production of recombinant chimeric triple helical collagen (see, e.g., PCT patent publication No. WO2010071938); fusion proteins with three α chain polypeptides (see, e.g., U.S. patent publication No. 20130237486); and stimulating fibroblast cells to express native collagen proteins (see, e.g., U.S. patent publication No. 20100239556).

### Animal Models for Collagen Associated Diseases

Mice with genetically engineered collagen mutations have proved valuable for defining the functions of various collagens and for studying many aspects of the related diseases and physiological functions of collagen. For example, COL4A3 knock-out mice are used as models for Alport syndrome (Cosgrove et al., Genes Dev., 1996, 10, 1403-1413).

According to the present invention, studies are designed to inject collagen IV, either extracted from collagen IV containing tissues, or produced by recombinant methods, intravenously or by any other suitable delivery routes, to mouse models of Alport syndrome. A comprehensive analysis of collagen IV incorporation into glomerular basement membrane (GBM), histological features of GBM and other collagen IV function assays such as collagen IV receptor binding, interaction with other GBM components, cell migration and differentiation and/or biomarker measurement, are conducted after administering collagen IV to mice with Alport-like syndromes. Following administration, mice treated with collagen IV replacement are analyzed for renal functions, such as urine analysis of hematuria, proteinuria, albumin-to-creatinine ratio, or estimated glomerular filtration rate.

### Collagen IV

Collagen IV is the most abundant protein found in extracellular basement membranes. There are six genetically distinct collagen IV α chains, α1 through α6 encoded by six genes COL4A1 to COL4A6, that assemble to form three different heterotrimers (referred to as protomers): α1-α1-α2, α3-α4-α5, and α5-α5-α6. The amino acid sequence of each a chain polypeptide is listed in Table 2, including their UniProt accession numbers (where more than one isoform is known, isoform 1 is shown). It is understood to one skilled in the art that the representative sequences also include any variants and derivatives that do not substantially change each polypeptide. Each collagen IV alpha chain can be divided into three domains: the 7S domain, a small non-collagenous N-terminal domain; a major collagenous domain in the middle region (about 1400 amino acid residues); and the NC1 domain, a non-collagenous globular domain constituting the C-terminal domain (about 230 residues).

Like all collagen chains, the collagenous domains of collagen IV chains contain numerous Gly-X—Y amino acid triplet repeats, where proline and hydroxyproline are frequently located at positions X and Y. The presence of glycine as each third amino acid is also essential, as it is the only amino acid small enough to fit into the center of the triple helix in collagenous proteins. However, unlike fibril-forming collagen of bone and cartilage, the Gly-X—Y repeat region of collagen IV displays multiple interruptions (i.e. about 20 short non-collagenous sequences), imparting flexibility to the collagen IV protomer and to the network that it forms in basement membranes.

The three α chains of collagen IV protomers are organized into triple helices in the 7S and the major collagenous domains, but in the NC1 domain each chain is folded into a globular structure, stabilized by intrachain disulfide bonds. During the assembly of the heterotrimer, the NC1 domains initiate a molecular interaction between three α chains, and protomer trimerization then proceeds in a zipper like format from the C-terminal end, resulting in a fully assembled protomer. Two collagen IV protomers form an end to end dimer through their C-terminal NC1 domains which forms a NC1 hexamer, and next, four protomers form tetramers through the dodecameric interactions of the N-terminal 7S domains and polymerize into complex collagen IV network. They are heavily linked via the disulfide bonds, unusual covalent sulfilimine (S=N) chemical bonds that cross-link methionine and hydroxylysine residues at the interface of adjoining triple helical protomers, and lysyl oxidase-mediated crosslinks (Borza et al., PNAS, 2014, 111(1), 331-336).

Collagen IV uniquely contains, among collagen types, sulfilimine bonds (S=N) (Fidler et al., Proc Natl Acad Sci USA. 2014, 111(1), 331-336), which are catalyzed by peroxidasin, an extracellular matrix associated peroxidase. The sulfimine bonds are located between pairs of trimeric NC1 domains, driving the formation of the collagen IV network (Vanacore et al., Science, 2009, 325, 1230-1234). In humans, peroxidasin is expressed most highly in the endothelium.

TABLE 2
Collagen IV α chains and Sequences

| | | UniProt | SEQ | |
|---|---|---|---|---|
| α | | accession | ID | |
| chain | Gene | No. | NO | Sequence |
| α1 | COL4A1 | P02462 | 1 | MGPRLSVWLLLLPAALLLHEEHSRAAAKGGCAGSGCG |
| | | | | KCDCHGVKGQKGERGLPGLQGVIGFPGMQGPEGPQGP |
| | | | | PGQKGDTGEPGLPGTKGTRGPPGASGYPGNPGLPGIPG |
| | | | | QDGPPGPPGIPGCNGTKGERGPLGPPGLPGFAGNPGPPG |
| | | | | LPGMKGDPGEILGHVPGMLLKGERGFPGIPGTPGPPGLP |
| | | | | GLQGPVGPPGFTGPPGPPGPPGPPGEKGQMGLSFQGPK |
| | | | | GDKGDQGVSGPPGVPGQAQVQEKGDFATKGEKGQKG |
| | | | | EPGFQGMPGVGEKGEPGKPGPRGKPGKDGDKGEKGSP |
| | | | | GFPGEPGYPGLIGRQGPQGEKGEAGPPGPPGIVIGTGPL |
| | | | | GEKGERGYPGTPGPRGEPGPKGFPGLPGQPGPPGLPVP |
| | | | | GQAGAPGFPGERGEKGDRGFPGTSLPGPSGRDGLPGPP |
| | | | | GSPGPPGQPGYTNGIVECQPGPPGDQGPPGIPGQPGFIGE |
| | | | | IGEKGQKGESCLICDIDGYRGPPGPQGPPGEIGFPGQPG |
| | | | | AKGDRGLPGRDGVAGVPGPQGTPGLIGQPGAKGEPGE |
| | | | | FYFDLRLKGDKGDPGFPGQPGMPGRAGSPGRDGHPGL |
| | | | | PGPKGSPGSVGLKGERGPPGGVGFPGSRGDTGPPGPPG |
| | | | | YGPAGPIGDKGQAGFPGGPGSPGLPGPKGEPGKIVPLPG |
| | | | | PPGAEGLPGSPGFPGPQGDRGFPGTPGRPGLPGEKGAV |
| | | | | GQPGIGFPGPPGPKGVDGLPGDMGPPGTPGRPGFNGLP |
| | | | | GNPGVQGQKGEPGVGLPGLKGLPGLPGIPGTPGEKGSI |
| | | | | GVPGVPGEHGAIGPPGLQGIRGEPGPPGLPGSVGSPGVP |
| | | | | GIGPPGARGPPGGQGPPGLSGPPGIKGEKGFPGFPGLDM |
| | | | | PGPKGDKGAQGLPGITGQSGLPGLPGQQGAPGIPGFPGS |
| | | | | KGEMGVMGTPGQPGSPGPVGAPGLPGEKGDHGFPGSS |
| | | | | GPRGDPGLKGDKGDVGLPGKPGSMDKVDMGSMKGQ |
| | | | | KGDQGEKGQIGPIGEKGSRGDPGTPGVPGKDGQAGQP |
| | | | | GQPGPKGDPGISGTPGAPGLPGPKGSVGGMGLPGTPGE |
| | | | | KGVPGIPGPQGSPGLPGDKGAKGEKGQAGPPGIGIPGLR |
| | | | | GEKGDQGIAGFPGSPGEKGEKGSIGIPGMPGSPGLKGSP |
| | | | | GSVGYPGSPGLPGEKGDKGLPGLDGIPGVKGEAGLPGT |
| | | | | PGPTGPAGQKGEPGSDGIPGSAGEKGEPGLPGRGFPGFP |
| | | | | GAKGDKGSKGEVGFPGLAGSPGIPGSKGEQGFMGPPGP |
| | | | | QGQPGLPGSPGHATEGPKGDRGPQGQPGLPGLPGPMGP |
| | | | | PGLPGIDGVKGDKGNPGWPGAPGVPGPKGDPGFQGMP |
| | | | | GIGGSPGITGSKGDMGPPGVPGFQGPKGLPGLQGIKGD |
| | | | | QGDQGVPGAKGLPGPPGPPGPYDIIKGEPGLPGPEGPPG |
| | | | | LKGLQGLPGPKGQQGVTGLVGIPGPPGIPGFDGAPGQK |
| | | | | GEMGPAGPTGPRGFPGPPGPDGLPGSMGPPGTPSVDHG |
| | | | | FLVTRHSQTIDDPQCPSGTKILYHGYSLLYVQGNERAH |
| | | | | GQDLGTAGSCLRKFSTMPFLFCNINNVCNFASRNDYSY |
| | | | | WLSTPEPMPMSMAPITGENIRPFISRCAVCEAPAMVMA |
| | | | | VHSQTIQIPPCPSGWSSLWIGYSFVMHTSAGAEGSGQAL |
| | | | | ASPGSCLEEFRSAPFIECHGRGTCNYYANAYSFWLATIE |
| | | | | RSEMFKKPTPSTLKAGELRTHVSRCQVCMRRT |
| α2 | COL4A2 | P08572 | 2 | MGRDQRAVAGPALRRWLLLGTVTVGFLAQSVLAGVK |
| | | | | KFDVPCGGRDCSGGCQCYPEKGGRGQPGPVGPQGYNG |
| | | | | PPGLQGFPGLQGRKGDKGERGAPGVTGPKGDVGARGV |
| | | | | SGFPGADGIPGHPGQGGPRGRPGYDGCNGTQGDSGPQ |
| | | | | GPPGSEGFTGPPGPQGPKGQKGEPYALPKEERDRYRGE |
| | | | | PGEPGLVGFQGPPGRPGHVGQMGPVGAPGRPGPPGPPG |
| | | | | PKGQQGNRGLGFYGVKGEKGDVGQPGPNGIPSDTLHPI |
| | | | | IAPTGVTFHPDQYKGEKGSEGEPGIRGISLKGEEGIMGF |
| | | | | PGLRGYPGLSGEKGSPGQKGSRGLDGYQGPDGPRGPK |
| | | | | GEAGDPGPPGLPAYSPHPSLAKGARGDPGFPGAQGEPG |
| | | | | SQGEPGDPGLPGPPGLSIGDGDQRRGLPGEMGPKGFIG |
| | | | | DPGIPALYGGPPGPDGKRGPPGPPGLPGPPGPDGFLFGL |
| | | | | KGAKGRAGFPGLPGSPGARGPKGWKGDAGECRCTEG |
| | | | | DEAIKGLPGLPGPKGFAGINGEPGRKGDRGDPGQHGLP |
| | | | | GFPGLKGVPGNIGAPGPKGAKGDSRTITTKGERGQPGV |
| | | | | PGVPGMKGDDGSPGRDGLDGFPGLPGPPGDGIKGPPGD |
| | | | | PGYPGIPGTKGTPGEMGPPGLGLPGLKGQRGFPGDAGL |
| | | | | PGPPGFLGPPGPAGTPGQIDCDTDVKRAVGGDRQEAIQ |
| | | | | PGCIGGPKGLPGLPGPPGPTGAKGLRGIPGFAGADGGPG |
| | | | | PRGLPGDAGREGFPGPPGFIGPRGSKGAVGLPGPDGSPG |
| | | | | PIGLPGPDGPPGERGLPGEVLGAQPGPRGDAGVPGQPG |
| | | | | LKGLPGDRGPPGFRGSQGMPGMPGLKGQPGLPGPSGQ |
| | | | | PGLYGPPGLHGFPGAPGQEGPLGLPGIPGREGLPGDRG |
| | | | | DPGDTGAPGPVGMKGLSGDRGDAGFTGEQGHPGSPGF |
| | | | | KGIDGMPGTPGLKGDRGSPGMDGFQGMPGLKGRPGFP |
| | | | | GSKGEAGFFGIPGLKGLAGEPGFKGSRGDPGPPGPPPVI |
| | | | | LPGMKDIKGEKGDEGPMGLKGYLGAKGIQGMPGIPGL |
| | | | | SGIPGLPGRPGHIKGVKGDIGVPGIPGLPGFPGVAGPPGI |
| | | | | TGFPGFIGSRGDKGAPGRAGLYGEIGATGDFGDIGDTIN |
| | | | | LPGRPGLKGERGTTGIPGLKGFFGEKGTEGDIGFPGITG |
| | | | | VTGVQGPPGLKGQTGFPGLTGPPGSQGELGRIGLPGGK |
| | | | | GDDGWPGAPGLPGFPGLRGIRGLHGLPGTKGFPGSPGS |
| | | | | DIHGDPGFPGPPGERGDPGEANTLPGPVGVPGQKGDQG |
| | | | | APGERGPPGSPGLQGFPGITPPSNISGAPGDKGAPGIFGL |
| | | | | KGYRGPPGPPGSAALPGSKGDTGNPGAPGTPGTKGWA |
| | | | | GDSGPQGRPGVFGLPGEKGPRGEQGFMGNTGPTGAVG |
| | | | | DRGPKGPKGDPGFPGAPGTVGAPGIAGIPQKIAVQPGT |
| | | | | VGPQGRRGPPGAPGEMGPQGPPGEPGFRGAPGKAGPQ |
| | | | | GRGGVSAVPGFRGDEGPIGHQGPIGQEGAPGRPGSPGL |
| | | | | PGMPGRSVSIGYLLVKHSQTDQEPMCPVGMNKLWSGY |
| | | | | SLLYFEGQEKAHNQDLGLAGSCLARFSTMPFLYCNPGD |
| | | | | VCYYASRNDKSYWLSTTAPLPMMPVAEDEIKPYISRCS |
| | | | | VCEAPAIAIAVHSQDVSIPHCPAGWRSLWIGYSFLMHT |
| | | | | AAGDEGGGQSLVSPGSCLEDFRATPFIECNGGRGTCHY |
| | | | | YANKYSFWLTTIPEQSFQGSPSADTLKAGLIRTHISRCQ |
| | | | | VCMKNL |
| α3 | COL4A3 | Q01955 | 3 | MSARTAPRPQVLLLPLLLVLLAAAPAASKGCVCKDKG |
| | | | | QCFCDGAKGEKGEKGFPGPPGSPGQKGFTGPEGLPGPQ |
| | | | | GPKGFPGLPGLTGSKGVRGISGLPGFSGSPGLPGTPGNT |
| | | | | GPYGLVGVPGCSGSKGEQGFPGLPGTPGYPGIPGAAGL |
| | | | | KGQKGAPAKGEDIELDAKGDPGLPGAPGPQGLPGPPGF |
| | | | | PGPVGPPGPPGFFGFPGAMGPRGPKGHMGERVIGHKGE |
| | | | | RGVKGLTGPPGPPGTVIVTLTGPDNRTDLKGEKGDKGA |
| | | | | MGEPGPPGPSGLPGESYGSEKGAPGDPGLQGKPGKDG |
| | | | | VPGFPGSEGVKGNRGFPGLMGEDGIKGQKGDIGPPGFR |
| | | | | GPTEYYDTYQEKGDEGTPGPPGPRGARGPQGPSGPPGV |
| | | | | PGSPGSSRPGLRGAPGWPGLKGSKGERGRPGKDAMGT |
| | | | | PGSPGCAGSPGLPGSPGPPGPPGDIVFRKGPPGDHGLPG |
| | | | | YLGSPGIPGVDGPKGEPGLLCTQCPYIPGPPGLPGLPGL |
| | | | | HGVKGIPGRQGAAGLKGSPGSPGNTGLPGFPGFPGAQG |
| | | | | DPGLKGEKGETLQPEGQVGVPGDPGLRGQPGRKGLDG |
| | | | | IPGTLGVKGLPGPKGELALSGEKGDQGPPGDPGSPGSP |
| | | | | GPAGPAGPPGYGPQGEPGLQGTQGVPGAPGPPGEAGPR |
| | | | | GELSVSTPVPGPPGPPGPPGHPGPQGPPGIPGSLGKCGD |
| | | | | PGLPGPDGEPGIPGIGFPGPPGPKGDQGFPGTKGSLGCP |
| | | | | GKMGEPGLPGKPGLPGAKGEPAVAMPGGPGTPGFPGE |
| | | | | RGNSGEHGEIGLPGLPGLPGTPGNEGLDGPRGDPGQPG |
| | | | | PPGEQGPPGRCIEGPRGAQGLPGLNGLKGQQGRRGKTG |
| | | | | PKGDPGIPGLDRSGFPGETGSPGIPGHQGEMGPLGQRG |
| | | | | YPGNPGILGPPGEDGVIGMMGFPGAIGPPGPPGNPGTPG |
| | | | | QRGSPGIPGVKGQRGTPGAKGEQGDKGNPGPSEISHVI |
| | | | | GDKGEPGLKGFAGNPGEKGNRGVPGMPGLKGLKGLPG |
| | | | | PAGPPGPRGDLGSTGNPGEPGLRGIPGSMGNMGMPGS |
| | | | | KGKRGTLGFPGRAGRPGLPGIHGLQGDKGEPGYSEGTR |
| | | | | PGPPGPTGDPGLPGDMGKKGEMGQPGPPGHLGPAGPE |
| | | | | GAPGSPGSPGLPGKPGPHGDLGFKGIKGLLGPPGIRGPP |
| | | | | GLPGFPGSPGPMGIRGDQGRDGIPGPAGEKGETGLLRA |
| | | | | PPGPRGNPGAQGAKGDRGAPGFPGLPGRKGAMGDAGP |
| | | | | RGPTGIEGFPGPPGLPGAIIPGQTGNRGPPGSRGSPGAPG |
| | | | | PPGPPGSHVIGIKGDKGSMGHPGPKGPPGTAGDMGPPG |
| | | | | RLGAPGTPGLPGPRGDPGFQGFPGVKGEKGNPGFLGSI |
| | | | | GPPGPIGPKGPPGVRGDPGTLKIISLPGSPGPPGTPGEPG |
| | | | | MQGEPGPPGPPGNLGPCGPRGKPGKDGKPGTPGPAGE |
| | | | | KGNKGSKGEPGPAGSDGLPGLKGKRGDSGSPATWTTR |
| | | | | GFVFTRHSQTTAIPSCPEGTVPLYSGFSFLFVQGNQRAH |
| | | | | GQDLGTLGSCLQRFTTMPFLFCNVNDVCNFASRNDYS |
| | | | | YWLSTPALMPMNMAPITGRALEPYISRCTVCEGPAIAIA |
| | | | | VHSQTTDIPPCPHGWISLWKGFSFIMFTSAGSEGTGQAL |
| | | | | ASPGSCLEEFRASPFLECHGRGTCNYYSNSYSFWLASL |
| | | | | NPERMFRKPIPSTVKAGELEKIISRCQVCMKKRH |
| α4 | COL4A4 | P53420 | 4 | MWSLHIVLMRCSFRLTKSLATGPWSLILILFSVQYVYGS |
| | | | | GKKYIGPCGGRDCSVCHCVPEKGSRGPPGPPGPQGPIGP |
| | | | | LGAPGPIGLSGEKGMRGDRGPPGAAGDKGDKGPTGVP |
| | | | | GFPGLDGIPGHPGPPGPRGKPGMSGHNGSRGDPGFPGG |
| | | | | RGALGPGGPLGHPGEKGEKGNSVFILGAVKGIQGDRGD |
| | | | | PGLPGLPGSWGAGGPAGPTGYPGEPGLVGPPGQPGRPG |
| | | | | LKGNPGVGVKGQMGDPGEVGQQGSPGPTLLVEPPDFC |
| | | | | LYKGEKGIKGIPGMVGLPGPPGRKGESGIGAKGEKGIPG |
| | | | | FPGPRGDPGSYGSPGFPGLKGELGLVGDPGLFGLIGPKG |
| | | | | DPGNRGHPGPPGVLVTPPLPLKGPPGDPGFPGRYGETG |
| | | | | DVGPPGPPGLLGRPGEACAGMIGPPGPQGFPGLPGLPGE |
| | | | | AGIPGRPDSAPGKPGKPGSPGLPGAPGLQGLPGSSVIYC |
| | | | | SVGNPGPQGIKGKVGPPGGRGPKGEKGNEGLCACEPGP |
| | | | | MGPPGPPGLPGRQGSKGDLGLPGWLGTKGDPGPPGAE |
| | | | | GPPGLPGKHGASGPPGNKGAKGDMVVSRVKGHKGER |
| | | | | GPDGPPGFPGQPGSHGRDGHAGEKGDPGPPGDHEDAT |
| | | | | PGGKGFPGPLGPPGKAGPVGPPGLGFPGPPGERGHPGV |
| | | | | PGHPGVRGPDGLKGQKGDTISCNVTYPGRHGPPGFDGP |
| | | | | PGPKGFPGPQGAPGLSGSDGHKGRPGTPGTAEIPGPPGF |
| | | | | RGDMGDPGFGGEKGSSPVGPPGPPGSPGVNGQKGIPGD |
| | | | | PAFGHLGPPGKRGLSGVPGIKGPRGDPGCPGAEGPAGIP |
| | | | | GFLGLKGPKGREGHAGFPGVPGPPGHSCERGAPGIPGQ |
| | | | | PGLPGYPGSPGAPGGKGQPGDVGPPGPAGMKGLPGLP |
| | | | | GRPGAHGPPGLPGIPGPFGDDGLPGPPGPKGPRGLPGFP |
| | | | | GFPGERGKPGAEGCPGAKGEPGEKGMSGLPGDRGLRG |
| | | | | AKGAIGPPGDEGEMAIISQKGTPGEPGPPGDDGFPGERG |
| | | | | DKGTPGMQGRRGELGRYGPPGFHRGEPGEKGQPGPPG |
| | | | | PPGPPGSTGLRGFIGFPGLPGDQGEPGSPGPPGFSGIDGA |
| | | | | RGPKGNKGDPASHFGPPGPKGEPGSPGCPGHFGASGEQ |
| | | | | GLPGIQGPRGSPGRPGPPGSSGPPGCPGDHGMPGLRGQP |
| | | | | GEMGDPGPRGLQGDPGIPGPPGIKGPSGSPGLNGLHGL |
| | | | | KGQKGTKGASGLHDVGPPGPVGIPGLKGERGDPGSPGI |
| | | | | SPPGPRGKKGPPGPPGSSGPPGPAGATGRAPKDIPDPGP |
| | | | | PGDQGPPGPDGPRGAPGPPGLPGSVDLLRGEPGDCGLP |
| | | | | GPPGPPGPPGPPGYKGFPGCDGKDGQKGPMGFPGPQGP |
| | | | | HGFPGPPGEKGLPGPPGRKGPTGLPGPRGEPGPPADVD |
| | | | | DCPRIPGLPGAPGMRGPEGAMGLPGMRGPPGPGCKGEP |
| | | | | GLDGRRGVDGVPGSPGPPGRKGDTGEDGYPGGPGPPG |
| | | | | PIGDPGPKGFGPGYLGGFLLVLHSQTDQEPTCPLGMPRL |
| | | | | WTGYSLLYLEGQEKAHNQDLGLAGSCLPVFSTLPFAYC |
| | | | | NIHQVCHYAQRNDRSYWLASAAPLPMMPLSEEAIRPY |
| | | | | VSRCAVCEAPAQAVAVHSQDQSIPPCPQTWRSLWIGYS |
| | | | | FLMHTGAGDQGGGQALMSPGSCLEDFRAAPFLECQGR |
| | | | | QGTCHFFANKYSFWLTTVKADLQFSSAPAPDTLKESQA |
| | | | | QRQKISRCQVCVKYS |
| α5 | COL4A5 | P29400 | 5 | MKLRGVSLAAGLFLLALSLWGQPAEAAACYGCSPGSK |
| | | | | CDCSGIKGEKGERGFPGLEGHPGLPGFPGPEGPPGPRGQ |
| | | | | KGDDGIPGPPGPKGIRGPPGLPGFPGTPGLPGMPGHDGA |
| | | | | PGPQGIPGCNGTKGERGFPGSPGFPGLQGPPGPPGIPGM |
| | | | | KGEPGSIIMSSLPGPKGNPGYPGPPGIQGLPGPTGIPGPIG |
| | | | | PPGPPGLMGPPGPPGLPGPKGNMGLNFQGPKGEKGEQ |
| | | | | GLQGPPGPPGQISEQKRPIDVEFQKGDQGLPGDRGPPGP |
| | | | | PGIRGPPGPPGGEKGEKGEQGEPGKRGKPGKDGENGQP |
| | | | | GIPGLPGDPGYPGEPGRDGEKGQKGDTGPPGPPGLVIPR |
| | | | | PGTGITIGEKGNIGLPGLPGEKGERGFPGIQGPPGLPGPP |
| | | | | GAAVMGPPGPPGFPGERGQKGDEGPPGISIPGPPGLDGQ |
| | | | | PGAPGLPGPPGPAGPHIPPSDEICEPGPPGPPGSPGDKGL |
| | | | | QGEQGVKGDKGDTCFNCIGTGISGPPGQPGLPGLPGPP |
| | | | | GSLGFPGQKGEKGQAGATGPKGLPGIPGAPGAPGFPGS |
| | | | | KGEPGDILTFPGMKGDKGELGSPGAPGLPGLPGTPGQD |
| | | | | GLPGLPGPKGEPGGITFKGERGPPGNPGLPGLPGNIGPM |
| | | | | GPPGFGPPGPVGEKGIQGVAGNPGQPGIPGPKGDPGQTI |
| | | | | TQPGKPGLPGNPGRDGDVGLPGDPGLPGQPGLPGIPGS |
| | | | | KGEPGIPGIGLPGPPGPKGFPGIPGPPGAPGTPGRIGLEGP |
| | | | | PGPPGFPGPKGEPGFALPGPPGPPGLPGFKGALGPKGDR |
| | | | | GFPGPPGPPGRTGLDGLPGPKGDVGPNGQPGPMGPPGL |
| | | | | PGIGVQGPPGPPGIPGPIGQPGLHGIPGEKGDPGPPGLDV |
| | | | | PGPPGERGSPGIPGAPGPIGPPGSPGLPGKAGASGFPGTK |
| | | | | GEMGMMGPPGPPGPLGIPGRSGVPGLKGDDGLQGQPG |
| | | | | LPGPTGEKGSKGEPGLPGPPGPMDPNLLGSKGEKGEPG |
| | | | | LPGIPGVSGPKGYQGLPGDPGQPGLSGQPGLPGPPGPKG |
| | | | | NPGLPGQPGLIGPPGLKGTIGDMGFPGPQGVEGPPGPSG |
| | | | | VPGQPGSPGLPGQKGDKGDPGISSIGLPGLPGPKGEPGL |
| | | | | PGYPGNPGIKGSVGDPGLPGLPGTPGAKGQPGLPGFPG |
| | | | | TPGPPGPKGISGPPGNPGLPGEPGPVGGGGHPGQPGPPG |
| | | | | EKGKPGQDGIPGPAGQKGEPGQPGFGNPGPPGLPGLSG |
| | | | | QKGDGGLPGIPGNPGLPGPKGEPGFHGFPGVQGPPGPP |
| | | | | GSPGPALEGPKGNPGPQGPPGRPGLPGPEGPPGLPGNG |
| | | | | GIKGEKGNPGQPGLPGLPGLKGDQGPPGLQGNPGRPGL |
| | | | | NGMKGDPGLPGVPGFPGMKGPSGVPGSAGPEGEPGLIG |
| | | | | PPGPPGLPGPSGQSIIIKGDAGPPGIPGQPGLKGLPGPQG |
| | | | | PQGLPGPTGPPGDPGRNGLPGFDGAGGRKGDPGLPGQP |
| | | | | GTRGLDGPPGPDGLQGPPGPPGTSSVAHGFLITRHSQTT |
| | | | | DAPQCPQGTLQVYEGFSLLYVQGNKRAHGQDLGTAGS |
| | | | | CLRRFSTMPFMFCNINNVCNFASRNDYSYWLSTPEPMP |
| | | | | MSMQPLKGQSIQPFISRCAVCEAPAVVIAVHSQTIQIPH |
| | | | | CPQGWDSLWIGYSFMMHTSAGAEGSGQALASPGSCLE |
| | | | | EFRSAPFIECHGRGTCNYYANSYSFWLATVDVSDMFSK |
| | | | | PQSETLKAGDLRTRISRCQVCMKRT |
| α6 | COL4A6 | Q14031 | 6 | MLINKLWLLLVTLCLTEELAAAGEKSYGKPCGGQDCS |
| | | | | GSCQCFPEKGARGRPGPIGIQGPTGPQGFTGSTGLSGLK |
| | | | | GERGFPGLLGPYGPKGDKGPMGVPGFLGINGIPGHPGQ |
| | | | | PGPRGPPGLDGCNGTQGAVGFPGPDGYPGLLGPPGLPG |
| | | | | QKGSKGDPVLAPGSFKGMKGDPGLPGLDGITGPQGAP |
| | | | | GFPGAVGPAGPPGLQGPPGPPGPLGPDGNMGLGFQGEK |
| | | | | GVKGDVGLPGPAGPPPSTGELEFMGFPKGKKGSKGEPG |
| | | | | PKGFPGLRGPPGFPGLGTTGEKGEKGEKGIPGLPGPRGP |
| | | | | MGSEGVQGPPGQQGKKGTLGFPGLNGFQGIEGQKGDI |
| | | | | GLPGPDVFIDIDGAVISGNPGDPGDPGLPGLKGDEGIQG |
| | | | | LRGPSGVPGLPALSGVPGALGPQGFPGLKGDQGNPGRT |
| | | | | TIGAAGLPGRDGLPGPPGPPGPPSPEFETETLHNKEAGFP |
| | | | | GLRGEQGPKGNLGLKGIKGDSGFCACDGGVPNTGPPG |
| | | | | EPGPPGPWGLIGLPGLKGARGDQGSGGAQGPAGAPGL |
| | | | | VGPLGPSGPKGKKGEPILSTIQGMPGDRGDSGSQGFRG |
| | | | | VIGEPGKDGVPGLPGLPGLPGDGGQGFPGEKGLPGLPG |
| | | | | EKGHPGPPGLPGNGLPGLPGPRGLPGDKGKDGLPGQQ |
| | | | | GLPGSKGITLPCIIPGSYGPSGFPGTPGFPGPKGSRGLPGT |
| | | | | PGQPGSSGSKGEPGSPGLVHLPELPGFPGPRGEKGLPGF |
| | | | | PGLPGKDGLPGMIGSPGLPGSKGATGDIFGAENGAPGE |
| | | | | QGLQGLTGHKGFLGDSGLPGLKGVHGKPGLLGPKGER |
| | | | | GSPGTPGQVGQPGTPGSSGPYGIKGKSGLPGAPGFPGIS |
| | | | | GHPGKKGTRGKKGPPGSIVKKGLPGLKGLPGNPGLVGL |
| | | | | KGSPGSPGVAGLPALSGPKGEKGSVGFVGFPGIPGLPGI |
| | | | | SGTRGLKGIPGSTGKMGPSGRAGTPGEKGDRGNPGPVG |
| | | | | IPSPRRPMSNLWLKGDKGSQGSAGSNGFPGPRGDKGEA |
| | | | | GRPGPPGLPGAPGLPGIIKGVSGKPGPPGFMGIRGLPGL |
| | | | | KGSSGITGFPGMPGESGSQGIRGSPGLPGASGLPGLKGD |
| | | | | NGQTVEISGSPGPKGQPGESGFKGTKGRDGLIGNIGFPG |
| | | | | KKGEDGKVGVSGDVGLPGAPGFPGVAGMRGEPGLPGS |
| | | | | SGHQGAIGPLGSPGLIGPKGFPGFPGLHGLNGLPGTKGT |
| | | | | HGTPGPSITGVPGPAGLPGPKGEKGYPGIGIGAPGKPGL |
| | | | | RGQKGDRGFPGLQGPAGLPGAPGISLPSLIAGQPGDPGR |
| | | | | PGLDGERGRPGPAGPPGPPGPSSNQGDTGDPGFPGIPGP |
| | | | | KGPKGDQGIPGFSGLPGELGLKGMRGEPGFMGTPGKV |
| | | | | GPPGDPGFPGMKGKAGPRGSSGLQGHPGQTPTAEAVQ |
| | | | | VPPGPLGLPGIDGIPGLTGDPGAQGPVGLQGSKGLPGIP |
| | | | | GKDGPSGLPGPPGALGDPGLPGLQGPPGFEGAPGQQGP |
| | | | | FGMPGMPGQSMRVGYTLVKHSQSEQVPPCPIGMSQLW |
| | | | | VGYSLLFVEGQEKAHNQDLGFAGSCLPRFSTMPFIYCNI |
| | | | | NEVCHYARRNDKSYWLSTTAPIPMMPVSQTQIPQYISR |
| | | | | CSVCEAPSQAIAVHSQDITIPQCPLGWRSLWIGYSFLMH |
| | | | | TAAGAEGGGQSLVSPGSCLEDFRATPFIECSGARGTCH |
| | | | | YFANKYSFWLTTVEERQQFGELPVSETLKAGQLHTRVS |
| | | | | RCQVCMKSL |
| α1 | NC1 | P02462 | 7 | GFLVTRHSQTIDDPQCPSGTKILYHGYSLLYVQGNERA |
| | domain | [1445- | | HGQDLGTAGSCLRKFSTMPFLFCNINNVCNFASRNDYS |
| | | 1669] | | YWLSTPEPMPMSMAPITGENIRPFISRCAVCEAPAMVM |
| | | | | AVHSQTIQIPPCPSGWSSLWIGYSFVMHTSAGAEGSGQ |
| | | | | ALASPGSCLEEFRSAPFIECHGRGTCNYYANAYSFWLA |
| | | | | TIERSEMFKKPTPSTLKAGELRTHVSRCQVCMRRT |
| α2 | NC1 | P08572 | 8 | GYLLVKHSQTDQEPMCPVGMNKLWSGYSLLYFEGQE |
| | domain | [1489- | | KAHNQDLGLAGSCLARFSTMPFLYCNPGDVCYYASRN |
| | | 1712] | | DKSYWLSTTAPLPMMPVAEDEIKPYISRCSVCEAPAIAI |
| | | | | AVHSQDVSIPHCPAGWRSLWIGYSFLMHTAAGDEGGG |
| | | | | QSLVSPGSCLEDFRATPFIECNGGRGTCHYYANKYSFW |
| | | | | LTTIPEQSFQGSPSADTLKAGLIRTHISRCQVCMKNL |
| α3 | NC1 | Q01955 | 9 | GFVFTRHSQTTAIPSCPEGTVPLYSGFSFLFVQGNQRAH |
| | domain | [1455- | | GQDLGTLGSCLQRFTTMPFLFCNVNDVCNFASRNDYS |
| | | 1669] | | YWLSTPALMPMNMAPITGRALEPYISRCTVCEGPAIAIA |
| | | | | VHSQTTDIPPCPHGWISLWKGFSFIMFTSAGSEGTGQAL |
| | | | | ASPGSCLEEFRASPFLECHGRGTCNYYSNSYSFWLASL |
| | | | | NPERMFRKPIPSTVKAGELEKIISRCQVCMKKR |
| α4 | NC1 | P53420 | 10 | GFLLVLHSQTDQEPTCPLGMPRLWTGYSLLYLEGQEKA |
| | domain | [1465- | | HNQDLGLAGSCLPVFSTLPFAYCNIHQVCHYAQRNDRS |
| | | 1690] | | YWLASAAPLPMMPLSEEAIRPYVSRCAVCEAPAQAVA |
| | | | | VHSQDQSIPPCPQTWRSLWIGYSFLMHTGAGDQGGGQ |
| | | | | ALMSPGSCLEDFRAAPFLECQGRQGTCHFFANKYSFWL |
| | | | | TTVKADLQFSSAPAPDTLKESQAQRQKISRCQVCVKYS |
| α5 | NC1 | P29400 | 11 | GFLITRHSQTTDAPQCPQGTLQVYEGFSLLYVQGNKRA |
| | domain | [1461- | | HGQDLGTAGSCLRRFSTMPFMFCNINNVCNFASRNDYS |
| | | 1685] | | YWLSTPEPMPMSMQPLKGQSIQPFISRCAVCEAPAVVIA |
| | | | | VHSQTIQIPHCPQGWDSLWIGYSFMMHTSAGAEGSGQ |
| | | | | ALASPGSCLEEFRSAPFIECHGRGTCNYYANSYSFWLAT |
| | | | | VDVSDMFSKPQSETLKAGDLRTRISRCQVCMKRT |
| α6 | NC1 | Q14031 | 12 | GYTLVKHSQSEQVPPCPIGMSQLWVGYSLLFVEGQEKA |
| | domain | [1467- | | HNQDLGFAGSCLPRFSTMPFIYCNINEVCHYARRNDKS |
| | | 1691] | | YWLSTTAPIPMMPVSQTQIPQYISRCSVCEAPSQAIAVH |
| | | | | SQDITIPQCPLGWRSLWIGYSFLMHTAAGAEGGGQSLV |
| | | | | SPGSCLEDFRATPFIECSGARGTCHYFANKYSFWLTTVE |
| | | | | ERQQFGELPVSETLKAGQLHTRVSRCQVCMKSL |

### Post-Translational Modifications

Similar to other collagen types, collagen IV molecules undergo extensive post-translational modification prior to secretion and this modification consists of the hydroxylation of appropriate proline and lysine residues, and the glycosylation of certain hydroxylysine residues to galactosylhydroxylysine and glucosylgalactosylhydroxylysine (reviewed in Bornstein and Sage, Annu. Rev. Biochem., 1980, 49, 957-1004). Collagen IV molecules may also be modified by the addition of asparagine-linked oligosaccharide side chains (Cooper et al., 1981; and Kurkinen et al., 1982). The extent of intracellular modifications in collagen IV is the highest among all the collagen types. Abnormal modification of collagen IV may affect the secretion of collagen IV (Wang et al., J. Bio. Chem., 1989, 264, 15556-15564).

Enzymes required for collagen IV modifications include prolyl-4 hydroxylase, prolyl-3-hydroxylase, lysyl hydroxylase, galactosyltransferase, glucosylgalactosyltransferase, and the asparagine-linked glycosylation machinery. Variants in the extent of modifications can also be found within the same type of collagen IV molecule, from different tissues, or even the same tissue in many physiological and pathological conditions (Kivirikko and Myllyla, Methods Enzymol., 1982, 82, 245-304).

Properly modified collagen IV is important for cell differentiation (such as F9 stem cells) (Wang et al., J. Bio. Chem., 1989, 264, 15556-15564).

Range of 3-hydroxyproline in Collagen IV is estimated to be between 6-16 3-hydroxyproline residues per 1000 amino acids (i.e. about 0.3% to 1.6%). Range of 4-hydroxyproline in Collagen IV is estimated to be 65-140 4-hydroxyproline residues per 1000 amino acids (i.e. about 6.5 to 14%) (see, e.g., Pokidysheva et al., Proc Natl Acad Sci USA. 2014, 111(1), 161-166; Tiainen et al., J Biol Chem. 2008, 283(28), 19432-19439; Price and Spiro, J Biol Chem., 1977, 252(23), 8697-9602; and Schuppen et al., Biochem J. 1984, 220(1), 227-233), The content of 4-hydroxyproline (4Hyp), 3-hydroxyproline (3Hyp), and hydroxylysine residues can influence collagen IV features. It is well established that 4-hydroxyproline residues stabilize the collagen triple helix through water-bridged intramolecular hydrogen bonding (Berg et al., Biochem. Biophys. Res. Commun., 1973, 52, 115-120). However, 3-hydroxyproline residues are much less abundant, as compared to 4-hydroxyproline residues. Only 1-2 residues of 3Hyp occur per chain in collagen types I and II and 3-6 residues occur per chain in collagen types V and XI. The content is highest in type IV collagen of basement membranes in which 10% of the total hydroxyproline can be 3Hyp (Gryder et al., J. Biol. Chem., 1975, 250, 2470-2474). It is also speculated that 3Hyp residues could be involved in fine-tuning of collagen triple helices through inter-triple-helical hydrogen bonds. Adequate 3-hydroxyprolination in collagen IV can reduce platelet aggregation.

### Basement Membranes (BMs)

The non-fibrillar assembly of collagen IV serves as a scaffold for forming the thin, sheet-like basement membrane with other matrix molecules, including subtypes of laminin, nidogen, and perlecan, a heparan sulfate proteoglycan (Breitkreutz D et al., Biomed. Res Int, 2013, e179784), as well as for cell attachment. Collagen IV α3-α4-α5 is mainly found in the basement membrane of kidney, inner ear and eye. Collagen IV α1-α1-α2 is the major macromolecule of the basement membrane of certain tissues.

As the principal structural elements of basement membranes, laminin and collagen IV form distinct networks which become non-covalently interconnected by mono- or oligomeric nidogen and perlecan. The collagen IV molecules are covalently cross-linked by disulfide bridges via their noncollagenous C- and globular N-terminus, giving rise to a very stable “chicken-wire”-like meshwork of high chemical resistance, which largely determines the mechanical strength of the BMs. In addition to the structural features, basement membranes also are important regulators for cell behavior, tissue compartmentalization, tissue remodeling and morphogenesis. Basement membranes are widely distributed extracellular matrices within cutaneous, muscle, ocular, vascular, neural tissue and kidney.

Collagen IV is primarily found in the basement membranes (BMs) of the skin, which form a barrier against environmental impacts. In the skin, collagen IV is synthesized by both epidermal keratinocytes and dermal fibroblasts. In the epidermal basement membrane, only collagen IV α1-α2-α2 and collagen IV α5-α5-α6 heterotrimers can be found (Hasegawa et al., Arch. Histol. Cytol. 2007, 70, 255-265). The inactivation of COL4A1 and COL4A2 also is incompatible with life, although only at later stages of gestation.

Collagen IV is also found in basement membrane of neurovascular bundles and other periodontium cells. It also plays role in maintaining the elastic system of the vasculature of the gums. For example, endothelial cells express collagen type IV for angiogenesis.

Glomerular Basement Membranes (GBMs):

In the kidney, GBMs are the central, non-cellular layers of the glomerular filtration barrier (GFB) that are situated between the two cellular components: endothelial cells and podocytes (unique epithelial cells). The GBM is composed primarily of four extracellular matrix macromolecules—laminin-521, collagen IV α3-α4-α5, the heparan sulfate proteoglycan (primarily agrin), and nidogen which are secreted by the endothelial cells and podocytes. These extracellular matrix proteins in the GBMs produce an interwoven meshwork thought to impart both size- and charge-selective properties.

During mammalian kidney development, collagen IV α1-α1-α2, the embryonic form of collagen IV present in the developing GBM, is normally replaced in the adult mature GBM by collagen IV α3-α4-α5. This isoform substitution occurs coincidentally with the transition of laminin chains in the GBM. It is hypothesized that the collagen IV transition might be required to accommodate the increased blood pressure in the adult, since α3-α4-α5 type IV collagen produces a more heavily cross-linked and more protease-resistant network compared to the α1-α1-α2 type IV collagen network.

Recent studies with improved microscopy techniques (e.g. STORM), uncovered the ultrastructure of the GBM and the distribution of collagen IV proteins in the kidney GBM. The GBM's collagen IV (α3-α4-α5) is secreted solely by podocytes (Abrahamson et al., J Am Soc Nephrol, 2009, 20, 1471-1479; and Abrahanson D R, Semin Nephrol, 2012, 32(4), 342-349) which eventually locate to the center of the GBM, away from podocytes (Suleiman et al., elife, 2013, e01149), suggesting that the GBM is permeable for the migration of collagen IV protomers from podocytes to the center of the GBM. It is shown that the endothelial fenestrae are about 100-150 nm, large enough to foster transport of large proteins such as collagen IV protomers which are rod-like heterotrimers with a diameter of about 12 nm. Other studies further demonstrated that the GBM is permeable to other large molecules that are larger than 400 kDa, such as ferritin and large antigen-antibody complexes (Farquhar et al., J Exp Med., 1961, 113, 47-66; Vogt et al., Kidney Int. 1982, 22(1): 27-35; and Fujigaki et al., Am J pathol., 1993, 142(3), 831-842). However, no evidence has been reported that exogenous collagen protein (such as recombinant collagen IV molecules) could be successfully transported to the GBM in the kidney via in vivo delivery. Furthermore, it is unanticipated that such exogenous collagen IV molecules can integrate into the GBM and form a correct basement network with other components of the GBM. Accordingly, the present invention will administer recombinant collagen IV protein, in particular collagen IV protomers, dimers, tetramers or multimers to the GBM sites that are impaired by collagen IV defects via systemic or local delivery. The collagen IV protomers, dimers, tetramers or multimers will then be embedded into the defective GBM and restore the normal matrix protein network in the GBM in the kidney.

Deficiencies in collagen IV, such as the absence of the α3-α4-α5 type IV collagen network, caused by mutations in the COL4A3, COL4A4 and/or COL4A5 genes, often impair basement membranes (e.g. GBM), causing many diseases including Alport syndrome, as well as several rheumatologic and dermatological diseases such as acquired epidermolysis bullosa, and the vascular complications of nephropathy and retinopathy in diabetes. Similarly, deficiencies in other components of the GBM (e.g., laminin and agrin) can impair basement membranes, causing nephrotic disease. For example, mutations in the laminin beta2 gene (LAMB2) cause Pierson syndrome, a rare autosomal recessive disease characterized by renal failure from nephrotic syndrome and diffuse mesangial sclerosis (Bull et al., J Pathol., 2014, 233(1), 18-26). Laminin β2 is one of the three chains of the heterotrimeric LAM-521 (α5β2γ1), the major laminin heterotrimer in the mature GBM.

### Alport Syndrome (AS)

Alport syndrome is an inherited disorder of glomerular basement membranes, resulting in progressive renal failure due to glomerulonephropathy. Alport syndrome typically presents in childhood as hematuria or proteinuria which may be associated with hearing loss and ocular dysfunction, and the disease gradually progresses to renal failure (such as end stage of renal disease (ESRD)) in adulthood. Renal biopsy test of patient's kidney confirms the absence of collagen IV alpha chains as well as pathological alterations of the GBM. Hearing loss and ESRD progress at near unity and the timing of stage of each symptom slightly varies per a genotype-phenotype correlation (see, e.g., Kashtan et al., J of Clinical Invest., 1999, 78, 1035-1044). Hearing loss, in some patients, is associated with renal pathology. Burke et al. (Burkee et al., Acta Ophthal., 1991, 69: 555-557) described bilateral corneal epithelial erosions in Alport syndrome. Patients may develop sensorineural hearing loss.

Ocular abnormalities have been observed in some Alport syndrome patients. Typical ocular associations are a dot-and-fleck retinopathy, which occurs in approximately 85% of affected adult males, anterior lenticonus, which occurs in approximately 25%, and rare posterior polymorphous corneal dystrophy. Govan et al described that anterior lenticonus (abnormal shape of lens) and retinal flecks in the macular and midperipheral retina as characteristic ophthalmic findings in Alport syndrome (Govan et al., Brit. J. Ophthal., 1983, 67: 493-503). The ocular manifestations were identical in the X-linked and autosomal forms of Alport syndrome. These abnormalities correlate with a defect in the collagen IV molecule.

The ultrastructural features on kidney biopsy that are diagnostic of Alport syndrome consist of (i) irregular thickening and thinning of the glomerular basement membrane (GBM); (ii) splitting or lamellation of the GBM; (iii) ‘basket weaving’ of the GBM and (iv) foot process fusion in regions of an abnormal GBM. Furthermore, the earliest ultrastructural finding in Alport syndrome is diffuse thinning of the GBM, which sometimes results in girls or women being misdiagnosed with thin basement membrane nephropathy (TBMN). The collagen IV α3, α4, and α5 chains are absent biochemically from the GBM of patients with Alport syndrome.

Although in Alport syndrome GBM embryonic collagen IV α1-α1-α2 continues to exist and is believed to delay the progression of disease, it is hypothesized that the anomalous persistence of these fetal isoforms in the GBM confers an increase in susceptibility to proteolytic attack by collagenases and cathepsins. Collagen IV α3-α4-α5 forms a more rigid disulfide network of hexamers and is more resistant to proteolytic degradation at the site of glomerular filtration. The absence of these potentially protective collagen IV isoforms in the GBM from Alport syndrome patients may explain the progressive basement membrane splitting and increased damage as the kidneys deteriorate in these patients.

Alport Syndrome is genetically heterogeneous, caused by mutations in the genes encoding the α3, α4 or α5 chain of collagen IV (COL4A3, COL4A4 and/or COL4A5). Mutations in COL4A3 and COL4A4 cause autosomal recessive Alport syndrome which account for ˜15% of Alport syndrome, while the COL4A5 mutations cause X-linked Alport syndrome which account for the remaining 85%. Autosomal dominant inheritance is rare. Some examples of mutations in COL4A3, COL4A4 and COL4A5 that cause Alport syndrome are listed in Table 3. More mutations in COL4A5 may be found in the COL4A5 database (http://www.arup.utah.edu/database/ALPORT/ALPORT_display.php).

It is important to distinguish between X-linked and autosomal recessive inheritance to properly assess the risk of renal failure in other family members. Autosomal recessive inheritance is suspected when disease occurs in a single generation and where female and male individuals are affected with equal frequency and severity. Molecular testing is often employed to confirm the clinical diagnosis.

Alport syndrome is also a feature of two other disorders caused by gene deletion involving COL4A5 gene: Alport syndrome and diffuse leiomyomatosis; and Alport syndrome, mental retardation, midface hypoplasia, and elliptocytosis.

X-Linked Alport Syndrome (XLAS):

eighty-five percent of Alport syndrome results from mutations in X-linked, COL4A5 gene encoding the α5-chain of collagen IV and is associated with hematuria, ocular abnormalities and high-tone sensorineural hearing loss. Nearly all affected males have decreased kidney function resulting in end-stage renal disease (ESRD) as early as the second decade of life. Affected females are too at risk for developing nephrotic syndrome, decreased kidney function and ESRD. Temporal macular thinning is also associated with XLAS (Ahmed et al., JAMA ophthalmol. 2013, 131(6), 777-782).

GBM lamellation is usually widespread in men with XLAS. The GBM is initially thinned in boys, but there is focal lamellation that becomes more extensive with time.

Immunostaining for the α3, α4 and α5 chains of collagen IV demonstrates the complete absence of these collagen chains in the GBM, distal tubular basement membrane (dTBM) and Bowman's capsule in essentially all males with XLAS, whereas women who are heterozygous carriers of XLAS demonstrate a segmental or ‘mosaic’ absence due to variable X-chromosome inactivation. These immunohistologic features help to distinguish XLAS from autosomal-recessive AS (ARAS), where expression of the α5 chain of collagen IV by immunostaining is negative in the GBM but positive in the dTBM and Bowman's capsule. The epidermal membrane of the skin also has no α5(IV) chain.

Mutations are different in each family with X-linked Alport syndrome, and more than 700 variants have been described (https://grenada.lumc.nl/LOVD2/COL4A/home.php?select_db=COL4A5). About 50% result in stop codon either directly or downstream, and 40% of mutations are missense. Large deletions/insertions, rearrangements, nonsense mutations and other genetic changes are also reported. Some examples of the identified mutations include missense mutations (G123E, Guo et al., Mol Biol Rep. 2014, 4196, 3631-3635); G1205V); nonsense mutations (Q379X); missense mutations in the collagenous domain of COL4A5; hypomorphic mutations (G624D, P628L; Pierides A et al., Hippokratia, 2013, 17(3), 207-213); complex deletion/insertion mutations (c.359_363delGTATTinsATAC) in the COL4A5 gene (Wang et al., Gene, 2013, 512(2), 482-485), mutations at splice sites; and deep intronic mutations in the COL4A5 gene (King K et al., Human Genet. 2002, 111, 548-554).

Autosomal recessive Alport syndrome (ARAS): about fifteen percent of Alport syndrome results from autosomal recessive homozygous or compound heterozygous mutations in both copies (in trans) of COL4A3 or COL4A4 genes (Mochizuki T et al., Nat. Genet., 1994, 8, 77-81). Mutations in COL4A3 or COL4A4 genes include missense changes, frameshift changes, small deletions/insertions, duplications, intronic variants, splicing mutations and nonsense mutations.

TABLE 3
Examples of collagen IV mutations and Alport syndrome

| Phenotype | Mutation |
|---|---|
| Alport Syndrome, Autosomal recessive | COL4A3, 5-BP DEL, NT4414 |
| Alport Syndrome, Autosomal recessive | COL4A3, ARG1481TER |
| Alport Syndrome, Autosomal recessive | COL4A3, SER1524TER |
| Alport Syndrome, Autosomal recessive | COL4A3, 5-BP DEL |
| Alport Syndrome, Autosomal recessive | COL4A3, EX5, C-T, ARG-TER |
| Alport Syndrome, Autosomal recessive | COL4A3, ALU INS, EX6 |
| Hematuria, benign familial | COL4A3, GLY1015GLU |
| Hematuria, benign familial | COL4A3, GLY985VAL |
| Alport Syndrome, Autosomal | COL4A3, IVS21DS, G-A, -1 |
| dominant | |
| Alport Syndrome, Autosomal | COL4A3, GLY1167ARG |
| dominant | |
| Alport Syndrome, X-linked | COL4A5, EX5-10DEL |
| Alport Syndrome, X-linked | COL4A5, CYS108SER |
| Alport Syndrome, X-linked | COL4A5, 10-15-KB INS, |
| | 40-KB DEL |
| Alport Syndrome, X-linked | COL4A5, 450-KB DEL |
| Alport Syndrome, X-linked | COL4A5, 38-KB DEL |
| Alport Syndrome, X-linked | COL4A5, GLY1143ASP |
| Alport Syndrome, X-linked | COL4A5, GLY325ARG |
| Alport Syndrome, X-linked | COL4A5, 3-PRIME AND |
| | PARTIAL 5-PRIME |
| | DELETION |
| Alport Syndrome, X-linked | COL4A5, TRP1538SER |
| Alport Syndrome, X-linked | COL4A5, GLY521CYS |
| Alport Syndrome, X-linked | COL4A5, GLY325GLU |
| Alport Syndrome, X-linked | COL4A5, GLY289VAL AND |
| | ARG1421CYS |
| Alport Syndrome, X-linked | COL4A5, GLY54ASP |
| Alport Syndrome, X-linked | COL4A5, CYS1638TYR |
| Alport Syndrome, X-linked | COL4A5, LEU1649ARG |
| Alport Syndrome, X-linked | COL4A5, ARG1677GLN |
| Alport Syndrome, Autosomal recessive | COL4A4, GLY1201SER |
| Alport Syndrome, Autosomal recessive | COL4A4, SER1238TER |
| Alport Syndrome, Autosomal recessive | COL4A4, ARG1377TER |
| Alport Syndrome, Autosomal recessive | COL4A4, CYS1641TER |
| Alport Syndrome, Autosomal recessive | COL4A4, PRO1572LEU |
| Hematuria, benign familial | COL4A4, GLY897GLU |
| Hematuria, benign familial | COL4A4, 1-BP INS, 3222A |
| Hematuria, benign familial | COL4A4, GLY960ARG |

Autosomal Dominant Alport Syndrome:

Autosomal dominant inheritance, resulting from heterozygous COL4A3 or COL4A4 variants, is very rare (van der Loop F T et al., Kidney Int., 2000, 58, 1870-1875.)

### Current Treatment of Alport Syndrome

There is no satisfactory and curative treatment available for Alport syndrome. Patients developing end stage renal disease (ESRD) are treated by hemodialysis, and also by kidney transplantation. However, about 5% of transplanted males develop Alport post-transplant anti-GBM nephritis and lose the transplanted kidneys. Many studies have focused on developing novel treatments that can slow or prevent the development of kidney failure.

Treatments of Alport syndrome patients to date primarily address proteinuria, including calcineurin inhibition with cyclosporine (see, e.g., Sigmundsson et al., Scand J Urol Nephrol, 2006, 40, 522-525) and the blockage of the renin-angiotensin aldosterone system (RAAS) by angiotensin-converting enzyme (ACE) inhibitors, angiotensin receptor blockers (ARBs) and aldosterone inhibitors. Recent evidence has shown that it can significantly delay the time to onset of renal replacement therapy and ESRD (See e.g. Noone and Licht, Pediatr Nephrol. 2013, 28, 1025-1036).

ACE inhibitors that have been used to treat Alport Syndrome patients include, but are not limited to, enalapril, fosinopril, lisinopril, quinapril. ACE inhibitors are relatively well tolerated by most individuals. Nevertheless, they are not free of side effects, and some patients should not use ACE inhibitors. The most common side effects are cough, elevated blood potassium levels, low blood pressure, dizziness, headache, drowsiness, weakness, abnormal taste (metallic or salty taste), and rash. The most serious, but rare, side effects of ACE inhibitors are kidney failure, allergic reactions, a decrease in white blood cells, and swelling of tissues (angioedema).

ARBs that have been used to treat Alport Syndrome patients include, but are not limited to, losartan and candesartan.

Some studies in Alport mouse model suggest that vasopeptidase inhibitors (e.g., AVE688) and 3-hydroxy-3-methylglutaryl-coenzyme (HMG-CoA) reductase inhibitors showed significant improvement in COL4A3−/− mice (Reviewed by Katayama et al., Searching for a treatment for Alport Syndrome using mouse models, World J Nephrol, 2014, 3(4): 230-236).

Because the downstream effect of the pathological proteinuria, together with complete activation of peroximal tubular epithelial cells (PTECs), often causes tubulointerstitium transmission via inflammation and fibrosis, treatment strategies that attempt to inhibit these processes are also employed to limit disease progression in Alport syndrome, in combination with above mentioned antiproteinuria therapies. These treatments may include chemokine receptor antagonists such as a CCR1 (chemokine (C-C motif) receptor 1) antagonist (e.g., BX471).

Researchers have focused on developing novel treatments for Alport syndrome. Such new treatments include gene therapy (see, e.g., review by Tryggvason et al., Kidney International., 1997, 51, 1493-1499), microRNA regulation (see, e.g., U.S. patent publication No. 20140100263; Gomez et al., Anti-microRNA-21 oligonucleotides prevent Alport nephropathy progression by stimulating metabolic pathways, J Clin Invest. 2015, 125(1): 141-156); stem cells (see, e.g., U.S. patent publication No. 20090214488); collagen metalloprotease inhibitors (see, e.g., U.S. patent publication Nos. 20080187508; 20090318511; 20110112076; and 20110014186); targeted therapy such as RAC1/CDC42 inhibitors (see, e.g., PCT patent publication No. 2014028059) and collagen IV receptor integrin inhibitors (see, e.g., U.S. Pat. No. 6,492,325); the content of each of which is herein incorporated by reference in their entirety.

Most recently, strategies to restore the normal collagen α3-α4-α5(IV) network in the GBM, by either cell- or gene-based therapy are proposed (Lin et al., J Am Soc nephrol., 2014, 25(4), 687-692).

### Collagen IV and Other Diseases

Recent studies have demonstrated that deficits in collagen IV protein are associated with many other diseases. Mutations in COL4A1 cause perinatal cerebral hemorrhage and porencephaly (Gould D B et al. Science, 2005, 308(5725), 1167-1171) and muscle-eye-brain disease (MEB) and Walker Warburg Syndrome (WWS) (Labelle-Dumais C et al. Plos Genet. 2011, 7(5), e1002062). MEB/WWS belong to a spectrum of autosomal recessive diseases characterized by ocular dysgenesis, neuronal migration defects, and congenital muscular dystrophy.

Mutations in COL4A2 cause intracerebral hemorrhage and leukoencephalopathy (hemorrahagic stroke) (Gunda B et al., J Neurol., 2014, 261(3), 500-503), and familial porencephaly and small vessel disease (Verbeek E et al., Eur. J. Hum. Genet., 2012, 20(8), 844-851). Mutations in COL4A5 and COL4A6 cause Alport syndrome with oesophageal leiomyomatosis.

Some deficits in functional collagen IV protein, in particular, α3, α4 and α5 chains, may also be associated with, but not limited to, familial microhematuria with thin basement membranes; microhematuria; thin basement membrane nephropathy (TBMN); nephrotic-range proteinuria; progressive renal insufficiency; glomerular hematuria, heavy or mild proteinuria, and diabetic nephropathy (DN).

A rare autoimmune kidney disease called Goodpasture syndrome (also known as anti-glomerular basement antibody disease) is mediated by autoantibodies against the NC1 domain of the α3(IV) chain. The binding of autoantibodies usually cause rapidly progressive glomerulonephritis (Olaru et al., J Immunology, 2013, 190, 1424-1432).

The important role of Collagen IV in the GBM and its tight association with various diseases raise the possibility of using collagen IV to treat diseases. For example, U.S. Pat. No. 7,183,383 discloses the use of collagen IV protein to recover a cellular function (e.g. Na⁺/K⁺ ATPase activity, oxygen consumption and integrin localization to the basal membrane) following a renal epithelial cell injury (e.g. toxin-induced injury and drug-induced injury). The methods include the step of contacting directly the injured cells with an effective amount of collagen IV protein.

### Permeability of Nephrotic GBM

Further studies demonstrated that nephritic GBM is more permeable to large molecules than the normal GBM (Farquhar and Palade, J Exp Med., 1061, 114, 699-716). For example, a study (Schneeberger et al., J Exp Med., 1974, 139(5), 1283-1302) has shown that gamma globulin in the blood, injected horse radish peroxidase and catalase (about 240 kDa), and ferritin (480 kDa) can penetrate into renal glomerulus in a rat model of autologous immune complex (AIC) nephritis. Fujigaki also demonstrated that ferritin-anti-ferritin immune complexes can translocate across the GBM in nephritis rats (Fujigaki et al., Am J pathol., 1993, 142(3), 831-842). It is further shown that the penetrated ferritin can be retained in the GBM for about 3 days. The increased permeability of the GBM could enhance the penetration of large molecules through the GBM. Collagen IV (α3-α4-α5) protomers are about 480 KDa and it is assumed that molecules around this size may be readily enter the nephritic GBM, such as the impaired GBM in Alport syndrome. According to the present invention, recombinant collagen IV molecules are systemically or locally delivered to a subject with the defective GBM equivalent to that in Alport syndrome. It is found that recombinant collagen IV can be transported to the GBM, where they form correct networks and interact with other components of the GBM, restoring the structure of the GBM and virtually the filtering function of the GBM in the kidney.

As discussed herein, the present invention provides methods for treating diseases characterized by one or more collagen IV deficiencies by adding recombinant collagen IV protein back to the body, in particular, the glomerular basement membrane in the kidney. The collagen IV replacement will be embedded into affected GBM and restore their functions. In particular, the invention relates to Alport syndrome caused by mutations in COL4A3, COL4A4 and COL4A5 genes which encode the α3(IV), α4(IV) and α5(IV) chain polypeptides. In the context of the present invention, the recombinant collagen IV protein may be protomers, dimers tetramers, and multimers, and the mixture thereof. A collagen IV protomer in accordance with the present invention is a heterotrimer of collagen IV α3-α4-α5, the heterotrimer mainly found in the glomerular basement membrane. Additionally a collagen IV protomer may be a heterotrimer of the chimeric α3(IV), α4(IV) and α5(IV) chains in each of which all or part of the NC1 domain is replaced with all or part of the NC1 domain of α1(IV) and α2(IV) chains.

In some aspects, the recombinant collagen IV protein of the present invention may be formulated as a pharmaceutical composition with other suitable excipients. Such pharmaceutical compositions are discussed below. In particular, the recombinant collagen IV is recombinant human collagen IV.

### Pharmaceutical Compositions

Provided in the present invention are pharmaceutical compositions comprising recombinant collagen IV protomers, dimers, tetramers, multimers and/or the mixture thereof and pharmaceutically acceptable excipients. Such pharmaceutical compositions are suitable for administration and/or injection into a human patient in need thereof. Such compositions are often formulated as to permit the active ingredients (i.e. recombinant collagen IV) to be effective, and which contains no additional components which are toxic to the subjects to which the formulation would be administered.

### Collagen IV Protein

In some embodiments, the active ingredients are collagen IV protomers, dimers, tetramers, multimers and/or the mixture thereof. In some aspects, the collagen IV is a procollagen comprising three α chain polypeptides selected from the group consisting of α1(IV), α2(IV), α3(IV), α4(IV), α5(IV), and α6(IV), wherein each a chain is encoded by gene COL4A1, COL4A2, COL4A3, COL4A4, COL4A5, and COL4A6.

In some aspects, said collagen IV protomer is a heterotrimer of one α3(IV) chain polypeptide, one α4(IV) chain polypeptide and one α5(IV) chain polypeptide, wherein the α3(IV) chain polypeptide comprises the amino acid sequence of SEQ ID NO. 3 and/or variants thereof the α4(IV) chain polypeptide comprises the amino acid sequence of SEQ ID NO. 4 and/or variants thereof and the α5(IV) chain polypeptide comprises the amino acid sequence of SEQ ID NO. 5 and/or variants thereof.

In some embodiments of the present invention, the recombinant collagen IV may comprise chimeric α(IV) polypeptides, in particular, chimeric α3(IV), α4(IV) and α(5) polypeptides. It has been shown that in Alport post-transplant nephritis (APTN), an aggressive form of anti-glomerular basement membrane disease that targets the allograft in transplanted patients with Alport syndrome, the alloantibodies in patients target alloepitopes within the NC1 domain of the α3(IV) chain and/or alloepitopes that depend on the quaternary structure of the NC1 hexamers of collage IV α3-α4-α5 protomer (Olaru et al., J Am Soc Nephrol. 2013, 24(6), 889-895). Furthermore, the NC1 domains of collagen IV α3-α4-α5 are the main autoantigens in Goodpasture syndrome, a rapidly progressive renal disease with lung hemorrhage. It is expected that the substitutes of the NC1 domains of the α3(IV), α4(IV) and/or α(5) chains will reduce the autoimmune reaction induced by the administration of the recombinant collagen IV.

In other embodiments, said collagen IV protomer is a heterotrimer comprising one, two or three chimeric collagen IV α polypeptides selected from the chimeric α3(IV), α4(VI) and α5(IV) polypeptides. As disclosed in the present invention, a chimeric α3(IV) chain polypeptide is a chimeric polypeptide in which all or part of the NC1 domain of the α3(IV) chain is replaced with all or part of the NC1 domain of the α1(IV) and/or α2(IV) chains. A chimeric α4(IV) chain polypeptide is a chimeric polypeptide in which all or part of the NC1 domain of the α4(IV) chain is replaced with all or part of the NC1 domain of the α1(IV) and/or α2(IV) chains. A chimeric α5(IV) chain polypeptide is a chimeric polypeptide in which all or part of the NC1 domain of the α5(IV) chain is replaced with all or part of the NC1 domain of the α1(IV) and/or α2(IV) chains.

As a non-limiting example, a recombinant collagen IV protomer comprises one chimeric α3(IV) chain polypeptide in which all or part of the NC1 domain of the α3(IV) chain is replaced by all or part of the NC1 domain of the α1(IV) chain polypeptide, one α4(IV) chain polypeptide and one α5(IV) chain polypeptide, wherein the three polypeptides form a triple helix. As another non-limiting example, a recombinant collagen IV protomer may comprise one chimeric α3(IV) chain polypeptide in which all or part of the NC1 domain of the α3(IV) chain is replaced by all or part of the NC1 domain of the α1(IV) chain polypeptide, one chimeric α4(IV) chain polypeptide in which all or part of the NC1 domain of the α4(IV) chain is replaced by all or part of the NC1 domain of the α2(IV) chain polypeptide, and one chimeric α5(IV) chain polypeptide in which all or part of the NC1 domain of the α5(IV) chain is replaced by all or part of the NC1 domain of the α1(IV) chain polypeptide, wherein the three polypeptides form a triple helix.

In some embodiments, said collagen IV protein of the present invention may be a dimer comprising two collagen IV protomer as disclosed above. In some aspects, two collagen IV protomers disclosed in the present invention may be dimerized via enzymatic and/or chemical dimerization, or through non-covalent association.

In some embodiments, the collagen IV protein used for the present invention may contain certain percentage of 3-hydroxyproline, 4-hydroxyproline and/or lysyl hydroxylysine residues. In some aspects, the collagen IV protein may contain about 6.5% to about 14% of 4-hydroxyprolines (i.e. between 65-140 4-hydroxyproline residues/1000 AA) and/or about 0.3% to about 1.6% of 3-hydroxyprolines (i.e. between 6-16 3-hydroxyproline residues/1000 AA).

In other aspects, said collagen IV protein is human collagen IV protein. Collagen IV used for treatment/replacement may be obtained from a variety of sources, including extraction and purification from tissues that contain collagen IV (e.g. human and other mammals). Collagen IV may also be produced via genetic engineering such as recombinant collagen IV, particularly human recombinant collagen IV.

In some embodiments, the collagen IV protein, including collagen IV α3-α4-α5 and/or chimeric collagen IV protomers, dimers, tetramers, multimers and/or the mixture thereof, is formulated as pharmaceutical compositions. Said pharmaceutical compositions comprising recombinant collagen IV are suitable to administering to a subject in need, such as an Alport syndrome patient.

### Purification of Collagen IV

Collagen IV protomers, dimers, multimers and/or the mixture thereof, can be extracted from collagen IV containing tissues, such as basement membranes, placenta, eye lens, etc. Basically, collagen preparation methods involve extraction with diluted organic acids, precipitation with salts, optional gelation and/or lyophilization, tangential filtration and purification, etc. (see, e.g., U.S. Pat. Nos. 4,148,664; 5,028,695; 5,670,369; 5,814,328; 7,964,704; the content of each of which is hereby incorporated by reference in their entirety). It is known in the art that different collagen types can be extracted and separated for their solubility in solution with different ionic strengths and pH. Many methods for extracting collagen IV are in accordance with the method of Sage et al. (J. Biol. Chem., 1979, 254 (19), 9893-9900), which involves solubilization of collagen by pepsin hydrolysis. JP Patent Publication No. 11-171898 (1999) discloses a technique of isolating a polymer fraction of collagen IV, the content of which is herein incorporated by reference in its entirety.

As used herein, the term “collagen IV-containing tissues” refers to any tissue that contains collagen IV, including but not limited to tendon, skin, cornea, bone, cartilage, teeth, intervertebral disc, fetal skin, cardiovascular system, basement membrane, placenta, eye lens and anchoring fibrils beneath any epithelia. Collagen IV is most abundantly in the epithelial and endothelial basal lamina, glomerular basement membranes, fetal membranes, blood vessels, placental basement membrane. It may also be found in small amounts in other tissues,

U.S. Pat. No. 5,436,135 describes an extraction process of collagen IV from human and/or animal placenta. Said method combines enzymatic digestion (e.g. pepsin) and acid pH treatment, and can extract uncontaminated collagen type IV with very high efficiency; the content of which is herein incorporated by reference in its entirety.

U.S. Pat. No. 7,396,912 described a method for extracting collagen from tissues using fermentation. Microorganisms such as bacteria, yeast are provided to the collagen containing tissues to ferment the tissues. Collagens extracted via fermentation have an increased purity, comprising mostly of well-preserved collagen monomers with natural configurations; the content of which is incorporated by reference in its entirety.

U.S. Pat. No. 7,741,441 describes methods for extracting collagen IV from lens capsule without contamination by other proteins and without degradation or denaturation. Such methods involve in using aqueous acid solution to extract collagen IV content from lens capsule without using enzyme treatment, the content of which is hereby incorporated by reference in its entirety.

In some embodiments, collagen producing cells such as fibroblast cells may be used to express collagen IV. It is discussed in the art that collagen producing cells (e.g., fibroblast cells) may be stimulated with different agents to increase collagen expression/synthesis, including collagen IV. See, e.g., PCT patent publication No. WO1995031473; WO2008070893 and WO2008070892, the content of each of which is incorporated by reference in their entirety.

Many references in the art disclose other methods for extracting and purifying other types of collagens from a variety of resources, some including collagen IV. Such methods may be employed if needed (see, e.g., U.S. Pat. Nos. 2,979,438; 5,064,941; 5,436,135; 5,814,328; 7,964,704; and U.S. patent publication Nos. 20140147400 and 20130123468). Other methods that stimulate the production of collagens (including collagen IV) from fibroblast cells may also be used if needed (see, e.g. U.S. patent publication Nos. 20100239556 and 20080306001).

### Production of Recombinant Collagen IV

Recombinant technologies may also be used to produce recombinant human collagen IV. Recombinant collagen IV may be produced by culturing suitable host cells to express the recombinant DNA encoding the same, which may be purified from culture media since collagen IV is secreted outside of cells. Various mammalian cell lines may be employed to express recombinant collagen IV because mammalian secretory pathways are known to facilitate the assembly and folding of biologically active proteins. Other hosts such as yeast cells, plant cells, insect cells and/or bacteria may also be used to produce recombinant collagen IV protein of the present invention.

In order to produce the secreted collagen IV that will be released into the culture supernatants, either the natural signal peptide of collagen IV is used, or a heterologous signal peptide, for example, a signal peptide derived from another secreted protein being efficient in the particular expression system is used. An example of such recombinant collagen protein is discussed in U.S. Pat. No. 8,470,555, which teaches a recombinant collagen protein having collagen triple helix structure comprising a signal peptide of human collectin; the content of which is herein incorporated by reference in its entirety.

In the context of the present invention, conventional molecular biology, recombinant DNA techniques and protein biochemistry are within the skills of the art. Such techniques are well explained in the literatures, e.g., Sambrook et al. (2001) Molecular Cloning: A Laboratory Manual. 3^{rd} ed. Cold Spring Harbor Laboratory Press: Cold Spring Harbor, N.Y.; Ausubel et al. eds. (2005) Current Protocols in Molecular Biology, John Wiley and Sons, Inc.: Hoboken, N.J.; Bonifacino et al. eds. (2005) Current Protocols in Cell Biology, Hoboken, N.J.; and Coligan et al. eds. (2005) Current Protocols in Protein Science, John Wiley and Sons, Inc.: Hoboken, N.J.

Nucleic acids that encode collagen IV α chain polypeptides may be cloned into any expression vectors that are suitable for expressing proteins. The general nature of the vectors is not crucial to collagen IV production in accordance with the present invention. In general, suitable expression vectors and expression constructs will be apparent to those skilled in the art. Suitable expression vectors may be based on plasmid and phages which may be either host specific, or engineered for other hosts of interest. Other suitable vectors may include cosmids, retroviruses, and many other vehicles. Other control and regulatory sequences such as promoter, operators, inducer, terminator and other sequences will be apparent to those skilled in the art. The vectors and constructs for producing recombinant collagen IV may be modified and/or engineered in any suitable manner. Suitable vectors may be selected as a matter of course by those skilled in the art according to the desired expression system.

Many methods well known in the art can be used to produce the collagen IV α chain polypeptides of the present invention. As a non-limiting example, one straightforward method may include steps of obtaining the nucleic acids encoding the collagen IV α chain polypeptides, inserting them into a suitable expression vector (e.g. plasmids), transforming a suitable host (e.g. mammalian cell lines), culturing the transformed host, and obtaining the polypeptide of the invention by any suitable means, such as fragmentation and centrifugation.

In some aspects, said three collagen IV α chain polypeptides may be inserted into a common vector. In other aspects, said three collagen IV α chain polypeptides may be inserted to separate vectors and then co-transformed into a host to express simultaneously.

Other suitable cloning methods will be apparent to those skilled in the art.

In accordance with the present invention, recombinant collagen IV may be produced in eukaryotic expression system including mammalian cells and glycoengineered yeast cells. As a non-limiting example, CHO cell lines are of choice because they offer well-characterized, selectable and amplifiable gene expression systems which facilitate high level protein expression. In addition, these cells are easy to manipulate as adherent or suspension cultures and exhibit relatively good genetic stability. CHO cells and recombinant proteins expressed in them have been extensively characterized and have been approved for use in clinical manufacturing by regulatory agencies.

Other cell lines may include human embryonic kidney cell line 293 (HEK293 cells), human fibroblasts. For example, HEK 293 cells may be stably transfected with vectors that express α3(IV), α4(IV), and α5(IV) chain polypeptides. Cell extracts and culture media of these transfected cells may be used to detect the assembly of collagen heterotrimers, for example via co-immmunoprecipitation of α3(IV), α4(IV), and α5(IV) chain polypeptides (e.g., Kobayashi et al., Kidney International., 2003, 64(6), 1986-1996; and Kobayashi and Uchiyama, Biomed Res., 2010, 31(6), 371-377).

It has been demonstrated that cells cultured in a vitamin C-free medium produce the single-chain collagen IV α polypeptide in a much larger amount than that of the type IV collagen protein (see Yoshikawa, K. et al., J. Biochem., 2001, 129, 929-936). In some aspects of the present invention, cells may be transfected with a single construct comprising a single a chain polypeptide such as α3 chain, and cultured in vitamin C free medium to produce α3 chain polypeptide only. Such α3 chain may be mixed with other two a chain polypeptides or chimeric polypeptides (i.e. α4 and α5) produced by the same way, to form the collagen IV heterotrimer.

Cultures suitable for any living cells may be useful for cultures of the present invention. Culture system may vary from prokaryotic expression systems (e.g., E. coli cells) up to eukaryotic expression systems (e.g., CHO cells and HEK293 cells).

Escherichia coli may be used to express recombinant expression of hydroxylated human collagen IV. The characterization of new prolyl and lysyl hydroxylase genes encoded by the giant virus mimivirus reveals a method for production of hydroxylated collagen. The coexpression of a human collagen type IV construct together with mimivirus prolyl and lysyl hydroxylases in Escherichia coli may produce hydroxylated collagen IV. The respective levels of prolyl and lysyl hydroxylation may be similar to the hydroxylation levels of native human collagen type IV. The distribution of hydroxyproline and hydroxylysine along recombinant collagen IV may also be similar to that of native collagen as determined by mass spectrometric analysis.

In some embodiments, host cells that are defective in native collagen IV expression, or expression of other collagens, either artificially or naturally, may be used to produce recombinant collagen IV of the present invention.

Collagen IV synthesis involves many unusual co-translational and post-translational modifications, as discussed above, including the formation of 4-hydroxyproline, 3-hydroxyproline, and hydroxylysine in —X-Pro-Gly-, -Pro-4Hyp-Gly-, and —X-Lys-Gly-sequences, respectively. In some embodiments, cells used to produce recombinant collagen IV protein may be engineered to express collagen prolyl 4-hydroxylases (P4Hs), prolyl 3-hydroxylases (P3Hs), and/or lysyl hydroxylases (LHs).

In some aspects, cells used to produce recombinant collagen IV may be co-transfected with constructs that contain nucleic acid sequences encoding prolyl-3 hydroxylase (P3H) and recombinant collagen IV α chains, respectively. The P3H will increase the content of 3-hydroxyproline of recombinant collagen IV, wherein the higher numbers of 3-hydroxyproline residues of recombinant collagen IV can reduce platelet induced aggregation. In other aspects, cells used to produce recombinant collagen IV may be co-transfected with constructs that contain nucleic acid sequences encoding prolyl-4 hydroxylase (P4H) and recombinant collagen IV α chains, respectively. The P4H will increase the content of 4-hydroxyproline of recombinant collagen IV, wherein the higher content of 4-hydroxyproline residues of recombinant collagen IV will increase collagen thermal stability and/or decrease susceptibility to proteolytic digestion.

In yet other aspects, cells used to produce recombinant collagen IV may be co-transfected with constructs that contain nucleic acid sequences encoding lysyl hydroxylases (LH) and recombinant collagen IV α chains, respectively. The LH will increase the content of lysyl hydroxylysine of recombinant collagen IV, wherein the higher content of lysyl hydroxylysine residues of recombinant collagen IV will further increase the stability and provide sites for glycosylation modification.

Collagen IV contains a unique sulfilimine (S=N) bond between a methionine sulfur and hydroxylysine nitrogen which could reinforce the collagen IV network. Peroxidasin, an enzyme found in basement membranes, catalyzes formation of the sulfilimine bond (Bhave et al., Nature Chem. Biol., 2012, 8, 784-790). According to the present invention, collagen IV protomers may be used as the active ingredients of the pharmaceutical compositions given its relative small size. In this context, cells used to produce recombinant collagen IV may be engineered to deplete peroxidasin, therefore preventing dimerization of collagen IV protomers. In other aspects, a peroxidasin inhibitor may be applied to the host cells to prevent the formation of the sulfilimine bonds during recombinant collagen IV protomer synthesis. The peroxidasin inhibitor may be a nucleic acid such as a siRNA or antisense nucleic acid that inhibits synthesis of peroxidasin; an antibody that binds specifically to peroxidasin; a peptide that is a fragment of peroxidasin or a peroxidasin substrate, a small molecule, and/or an anion such as iodide or thiocyanate Inhibition of peroxidasin may also occur by removal of bromide in cultured cells or by application of a neutralizer of hypochlorous acid and/or hypobromous acid such as taurine.

In some embodiments, such cell systems may be used to produce the chimeric α(IV) chain polypeptides selected from the chimeric α3(IV), α4(VI) and α5(IV) polypeptides. The chimeric α3(IV) chain polypeptide may be encoded by a chimeric cDNA in which a nucleic acid sequence that encodes the amino acid sequence of all or part of the NC1 domain of the α3(IV) chain is replaced with a nucleic acid sequence that encodes the amino acid sequence of all or part of the NC1 domain of the α1(IV) and/or α2(IV) chains. The chimeric α4(IV) chain polypeptide may be encoded by a chimeric cDNA in which a nucleic acid sequence that encodes the amino acid sequence of all or part of the NC1 domain of the α4(IV) chain is replaced with a nucleic acid sequence that encodes the amino acid sequence of all or part of the NC1 domain of the α1(IV) and/or α2(IV) chains. The chimeric α5(IV) chain polypeptide may be encoded by a chimeric cDNA in which a nucleic acid sequence that encodes the amino acid sequence of all or part of the NC1 domain of the α5(IV) chain is replaced with a nucleic acid sequence that encodes the amino acid sequence of all or part of the NC1 domain of the α1(IV) and/or α2(IV) chains.

In other aspects, said chimeric cDNAs encoding chimeric α(IV) polypeptides may be codon optimized for expression in mammalian cells, bacteria, insects, plant cells and/or yeast. Codon optimization is well known in the art for optimizing expression of recombinant polypeptides.

Said chimeric cDNAs may be transfected into mammalian cells, bacteria, insect cells, plant cells and/or yeast to produce chimeric α(IV) polypeptides. Also provided in the present invention are transformed host cells, bacteria, insects, plant cells and/or yeasts that contain the chimeric cDNA encoding chimeric α(IV) polypeptides.

In some embodiments, the recombinant collagen IV protein of the present invention may further contain non-natural amino acids and/or other amino acid substitutes, such as those that may enhance the stability of a polypeptide.

### Pharmaceutically Acceptable Excipients

In some embodiments, the pharmaceutical compositions of the present invention may further comprise other pharmaceutically acceptable excipients.

The term “pharmaceutically acceptable excipient” refers to any ingredient having no therapeutic activity and having acceptable toxicity such as buffers, solvents, tonicity agents, stabilizers, antioxidants, surfactants or polymers used in formulating pharmaceutical products. They are generally safe for administering to humans according to established governmental standards, including those promulgated by the United States Food and Drug Administration.

Buffers:

As used herein, the term “buffer” encompasses those agents which maintain the solution pH in an acceptable range. A buffer is an aqueous solution consisting of a mixture of a weak acid and its conjugate base or a weak base and its conjugate acid. Its pH changes very little when a small amount of strong acid or base is added to it and thus it is used to prevent any change in the pH of a solution. Buffer solutions are used in collagen IV protein formulations as a means of keeping proteins stable within a narrow pH range.

A buffer can stabilize the pH of a pharmaceutical composition. Suitable buffers are well known in the art and can be found in the literature. Preferred pharmaceutically acceptable buffers comprise, but are not limited to, histidine-buffers, arginine-buffers, citrate-buffers, succinate-buffers, acetate-buffers and phosphate-buffers or mixtures thereof. Most preferred buffers comprise citrate, L-arginine, L-histidine or mixtures of L-histidine and L-histidine hydrochloride. Other preferred buffer is acetate buffer. Independently from the buffer used, the pH can be adjusted with an acid or a base known in the art, e.g. hydrochloric acid, acetic acid, phosphoric acid, sulfuric acid and citric acid, sodium hydroxide and potassium hydroxide. The pH is adjusted in range to provide acceptable stability, to maintain the solubility and insulinotropic activity of the collagen IV protomer, dimer, tetramer and/or multimer, and be acceptable for parenteral administration. The pH may be from about pH 4 to about pH 7.0, or about pH 5 to about pH 6, such as about pH 5, about pH 5.5, about pH 6, about pH 6.5, or about pH 7.0.

Tonicity Agents:

The term “tonicity agent”, as used herein, recites pharmaceutically acceptable excipient used to modulate the tonicity of a pharmaceutical composition and formulation. Tonicity in general relates to the osmotic pressure of a solution usually relative to that of human blood serum. Osmotic pressure is the pressure that must be applied to a solution to prevent the inward flow of water across a semi-permeable membrane. Osmotic pressure and tonicity are influenced only by solutes that cannot cross the membrane, as only these exert an osmotic pressure. A formulation can be hypotonic, isotonic or hypertonic, but is typically preferably isotonic. An isotonic formulation is liquid or liquid reconstituted from a solid form, e.g. from a lyophilized form and denotes a solution having the same tonicity as some other solution with which it is compared, such as physiologic salt solution and the blood serum.

Tonicity agent excipients are added to injectable, ocular or nasal preparations to reduce local irritation by preventing osmotic shock at the site of application. For comfort during administration, many injectable dosage forms must have the same salt (isotonic) concentration as the normal cells of the body and the blood.

Suitable tonicity agents include sugars, salts and amino acids. Some examples of tonicity agents include, but are not limited to, corn syrup, hydrous dextrose, anhydrous dextrose, trehalose, sucrose, glycerin, arginine, mannitol, potassium chloride and sodium chloride.

The term “sugar” as used herein denotes a monosaccharide or an oligosaccharide, which is water soluble. A monosaccharide is a monomeric carbohydrate which is not hydrolysable by acids, including simple sugars and their derivatives. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a chain. The monomeric saccharide units within an oligosaccharide can be identical or different. Examples of oligosaccharides include sucrose, trehalose, lactose, maltose and raffinose.

The term “amino acid” in context with tonicity agent or stabilizer, denotes a pharmaceutically acceptable organic molecule possessing an amino moiety located at an α-position to a carboxylic group. Examples of amino acids include arginine, glycine, ornithine, lysine, histidine, glutamic acid, asparagic acid, isoleucine, leucine, alanine, phenylalanine, tyrosine, tryptophane, methionine, serine, proline. Preferred amino acid in context with tonicity agent or stabilizer is arginine, tryptophane, methionine, histidine or glycine. For example, arginine is a protein solubilizer and also a stabilizer that reduces collagen IV aggregation.

Inorganic salts are effective tonicity agents and also commonly used as protein stabilizers. Inorganic salts may include, but are not limited to, sodium chloride (NaCl), sodium sulfate (Na₂SO₄), sodium thiocyanate (NaSCN), magnesium chloride (MgCl₂), magnesium sulfate (MgSO₄), ammonium thiocyanate (NH₄SCN), ammonium sulfate ((NH₄)₂SO₄), ammonium chloride (NH₄Cl), calcium chloride (CaCl₂), calcium sulfate (CaSO₄), zinc chloride (ZnCl₂) and the like, or combinations thereof.

It is well known that if a formulation requires a high concentration of one or more sugars to stabilize a protein, the inorganic salt concentration should be zero or kept very low in order to maintain the formulation's osmolality such that injection pain is reduced upon administration. In some embodiments, the collagen IV formulations are non-salt formulations in which inorganic salts are substantially excluded from addition to the formulations described herein. These non-salt formulations may maintain the osmolality of the collagen IV formulations with increased stability, and reduced phase change, such as precipitation or aggregation. It will be understood by those skilled in the art that the presence of inorganic salts within the presently disclosed formulations that are introduced by pH adjustment are not considered to be added inorganic salts.

In other embodiments, if a high concentration of collagen IV protein is not desired, the pharmaceutical compositions comprising collagen IV protein may be in any of a variety of physiologically acceptable salt forms, and/or with an acceptable pharmaceutical carrier and/or additives. Pharmaceutically acceptable salts include, e.g., acetate, benzenesulfonate, benzoate, bicarbonate, bitartrate, bromide, calcium edetate, camsylate, carbonate, chloride, citrate, di hydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate (embonate), pantothenate, phosphate/disphosphate, polygalacturonate, salicylate, stearate, subacetate, succinate, sulfate, tannate, tartrate, and teoclate/triethiodide anions; benzathine, chloroprocaine, choline, diethanolamine, ethylenediamine, meglumine, and procaine (organic) cations; and aluminium, calcium, lithium, magnesium, potassium, sodium, and zinc (metallic) cations. Pharmaceutically acceptable salts also include those salts described in, e.g., Berge et al., J. Pharm. Sci, 1977, 66, 1-19.

In some embodiments of the present invention, the collagen IV composition may further comprise mannitol as an isotonicity agent. The mannitol concentration is in the range of about 3.0 to about 6.3% w/v.

Surfactant: surfactants may be used to protect protein formulations against mechanical stresses like agitation and shearing without causing denaturation of the collagen IV protein, and also to reduce the adsorption on the surfaces during processing and storage. Surfactants may include, but are not limited to, poloxamers, polysorbates, polyoxyethylene alkyl ethers (Brij), alkylphenylpolyoxyethylene ethers (Triton-X) or sodium dodecyl sulphate (SDS). Preferred surfactants are polysorbates and poloxamers.

Polysorbates are oleate esters of sorbitol and its anhydrides, typically copolymerized with ethylene oxide. Commonly used polysorbates including Polysorbate 20 (poly(ethylene oxide) (20) sorbitan monolaurate, Tween 20) or Polysorbate 80 (poly(ethylene oxide) (80) sorbitan monolaurate, Tween 80), and Pluronic® polyols, can stabilize protein during processing and storage by reducing interfacial interaction and prevent protein from adsorption.

In some embodiments of the present invention, the collagen IV compositions may further comprise polysorbate-80 as a solubilizer and/or stabilizer. The concentration of polysorbate-80 is in the range of about 0.01 to 0.05% (w/v) (or expressed in terms of mg/ml, about 0.1 to 0.5 mg/mL). This concentration of polysorbate-80 is determined in combination with the collagen IV protein and mannitol to minimize the formation of soluble aggregates and insoluble particles.

Poloxamer means non-ionic triblock copolymers composed of a central hydrophobic chain of polypropylene oxide) (PPO) flanked by two hydrophilic chains of poly(ethylene oxide) (PEO), each PPO or PEO chain can be of different molecular weights.

Amounts of surfactants effective to provide stable high concentration collagen IV formulations are usually in the range of about 50 ppm to about 200 ppm. The collagen IV protein formulations of the present invention include, without limitation, formulations having one or more non-ionic surfactant(s) including, for example, one or more polysorbate(s), such as polysorbate 20 or 80; one or more polyoxamers, such as poloxamer 184 or 188; one or more Pluronic® polyol(s); and/or one or more ethylene/polypropylene block polymer(s). Exemplified herein are formulations having a polysorbate, such as polysorbate 20 (Tween 20) or polysorbate 80 (Tween 80).

Antioxidant:

Antioxidant may be used to prevent oxidation of the active pharmaceutical ingredient, in particular, the recombinant collagen IV protein. This includes chelating agents, reactive oxygen scavengers and chain terminators. Antioxidants include, but are not limited to, EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In particular, metal chelators such as EDTA, ALA, BAPTA, EGTA, DTPA and DMSA may be used to inhibit lysyl oxidase mediated collagen IV cross-linking among collagen IV protomers, dimers and/or multimers.

Collagen IV proteins may be produced as powder, suitable for solution and infusion, or formulated as solutions suitable for injection and other administration routes of such collagen IV proteins.

In some embodiments, the pharmaceutical composition of the present invention may contain a high concentration of collagen IV protein without loss of the stability of recombinant protein.

Standard pharmaceutical formulation techniques are well known to those skilled in the art (see, e.g., 2005, Physicians' Desk Reference®, Thomson Healthcare: Montvale, N.J., 2004; Remington: The Science and Practice of Pharmacy, 20th ed., Gennado et al., Eds. Lippincott Williams & Wilkins: Philadelphia, Pa., 2000). Suitable pharmaceutical additives include those discussed above, e.g., mannitol, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The compositions may also contain pH buffering reagents and wetting or emulsifying agents. The compositions may or may not contain preservatives.

The formulation of pharmaceutical compositions may vary depending on the intended routes of administration and other parameters (see, e.g., Rowe et al., Handbook of Pharmaceutical Excipients, 4th ed., APhA Publications, 2003). In some embodiments, the composition may be a sterile, non-pyrogenic, white to off-white lyophilized cake or powder to be administered by intravenous injection upon reconstitution with sterile water for injection. In other embodiments, the formulation itself may be a sterile, non-pyrogenic solution.

Lyophilized formulation:

In some embodiments, the pharmaceutical composition of the present invention may be formulated as lyophilized mixture, in the presence of lyoprotectant.

In other embodiments, the pharmaceutical composition of the present invention may be encapsulated in biodegradable polymers.

Aqueous formulation:

As used herein, the term “aqueous formulation” refers to a solution or liquid preparation that contains collagen IV protein in combination with one or more excipients (e.g., chemical additives) dissolved in a suitable solvent. In some embodiments, the collagen IV composition may be formulated as stable aqueous formulation comprising an effective amount of soluble collagen IV protein, a buffer such as a citrate-phosphate or citrate buffer with a desired pH, sucrose or trehalose, sodium chloride and either L-histidine or L-aspartic acid.

In some embodiments, formulations of collagen IV protein may contain, among others, excipients which inhibit adsorption, prevent oxidation, maintain pH, stabilize the collagen IV protein and control the osmolality of the pharmaceutical composition. In general, excipients that stabilize collagen IV can be chosen on the basis of the mechanisms by which they stabilize proteins against various chemical and physical stresses that could occur during a manufacturing process, under particular storage conditions, or associated with a particular mode of administration.

The concentration or amount of an excipient to use in a formulation will vary depending on, for example, the amount of collagen IV protein included in the formulation, the amount of other excipients included in the desired formulation, the amount or volume of other components in the formulation and the desired tonicity or osmolality that is desired to be achieved. In various embodiments, different types of excipients can be combined in a single formulation. Accordingly, a single formulation can contain a single excipient, two, three or more different types of excipients. The use of excipients in liquid formulations is an established practice to stabilize proteins against degradation or aggregation processes attributed for instance, to stresses that occur during manufacturing, shipping, storage, pre-use preparation, or administration. In practice, the presence of a particular excipient in a formulation may have more than one effect or purpose.

A variety of publications and reviews are available on protein stabilization, e.g. Arakawa, et al, Pharm. Res., 1991, 8(3), 285-91 (1991); Kendrick, et al, Pharmaceutical Biotechnology, 2002, 13, 61-84, and Randolph, et al., Pharmaceutical Biotechnology, 2002, 13, 159-175, the content of each of which is herein incorporated by reference in their entirety.

Accordingly, a variety of references in the art discuss protein formulations for pharmaceutical purposes, see, e.g., U.S. Pat. Nos. 6,821,515; 6,685,940; 8,420,081; and 8,613,919; and U.S. patent publication No. 20120294866; and 20130156760; and PCT patent publication No. WO2013096791; the content of each of which is herein incorporated by reference in their entirety.

In one embodiment, the collagen IV protein formulation of the present invention comprises collagen IV protomer, dimer, tetramer, multimer and/or the mixture thereof, wherein the collagen IV protomer is a heterotrimer comprising three α chain polypeptides selected from collagen IV α1, α2, α3, α4, α5 and α6 chains. In a preferred embodiment, said collagen IV protomer is a heterotrimer consisting of one α3 chain, one α4 chain and one α5 chain polypeptide.

In some aspects, the collagen IV formulations contain recombinant collagen IV protein comprising α3 (IV) chain polypeptide comprising the amino acid sequence of SEQ ID NO. 3 and/or variants thereof, α4 (IV) chain polypeptide comprising the amino acid sequence of SEQ ID NO.4 and/or variants thereof, α5 (IV) chain polypeptide comprising the amino acid sequence of SEQ ID NO. 5 and/or variants thereof. In other aspects, the collagen IV formulations contain collagen IV protein comprising chimeric α (IV) chain polypeptides selected from chimeric α3 (IV) chain polypeptide, chimeric α4 (IV) chain polypeptide and chimeric α5 (IV) chain polypeptide.

As a non-limiting example, a collagen IV protein formulation in accordance with the present invention may contain a pharmaceutically effective amount of collagen IV protein (e.g. recombinant human collagen IV protein), suitable concentration of a non-ionic surfactant, one or more amino acids selected from histidine, arginine, lysine, glycine and alanine, polysorbate-80, and/or one or more sugars selected from selected from mannitol, dextrose, glucose, trehalose and sucrose, wherein the concentration of collagen IV protein is from about 10 ng/ml to about 10 mg/ml, and wherein said collagen IV protein formulation has a pH of pH 4.5 to pH 6.5 and wherein said collagen IV protein formulation contains substantially no inorganic salt.

In a further embodiment, the collagen IV formulations may further include a metal chelator such as EDTA to inhibit cross linking of collagen IV protomers, dimers, multimers and the mixture thereof.

### Administration and Dosage

According to the present invention, recombinant human collagen IV protein, pharmaceutical compositions comprising collagen IV protein, or collagen IV protein formulations may be administered to a patient in need by intravenous injection, and/or other systemic or local administrations, such as intramuscular, subcutaneous, intracerebral, intracerebral ventricular, intracranial, intraocular, intra-aural delivery and delivery by acutely or chronically placed catheters.

The administration route of the pharmaceutical compositions of the present invention is preferably a parenteral route including intravenous, subcutaneous, intraperitoneal, and intramuscular routes. Intravenous administration is preferred. In addition to injection, implants and transdermal patches may be used, or an active compound may be prepared using a controlled-release preparation (see Sustained and Controlled Release Drug Delivery Systems, J. R. Robinson ed., Marcel Dekker, Inc., New York, 1978) including microcapsule delivery systems. A biodegradable or biocompatible polymer can be used, such as ethylene-vinyl acetate, polyethylene glycol (PEG), polyanhydride, polyglycolic acid, collagen, polyorthoester, or polylactic acid.

The dosage form of the pharmaceutical composition is not particularly limited. The pharmaceutical drug is, for example, in any of liquid, semisolid, and solid dosage forms. Specific examples thereof include solutions (e.g., injectable solutions and insoluble solutions), dispersions, suspensions, tablets, pills, powders, liposomes, and nanoparticles.

The dosage form is appropriately selected according to an administration route or indications. An injectable dosage form is preferred. Examples of preferable composition of the injectable dosage form include dosage forms of injectable solutions or insoluble solutions and specifically include those suitable for intravenous, subcutaneous, and intramuscular injection, preferably intravenous injection.

In addition, the pharmaceutical compositions of the present invention can be in any of solution, microemulsion, dispersion, liposome forms and nanoparticles, and other forms suitable for administration without limitations as long as the pharmaceutical drug is sterile and stable under production and storage conditions. The collagen IV protomer, dimer, tetramer, multimer, and/or mixtures thereof, is incorporated in a necessary amount of an appropriate solvent, if necessary together with one or the combination of the ingredients listed above. Subsequently, the mixture can be sterilized by filtration to prepare an injectable sterile solution.

In general, the pharmaceutical compositions are incorporated in a sterile medium containing a basic dispersion medium and necessary additional ingredient(s) listed above to prepare a dispersion. In the case of a sterile powder for preparing the injectable sterile solution, a preferable preparation method involves obtaining, by vacuum drying and freeze drying, a powder of an active ingredient with arbitrary desired additional ingredients from the solution already sterilized by filtration. For example, a particle size necessary for a dispersion can be maintained by use of a coating agent such as lecithin, while the appropriate flowability of a solution can be maintained by use of a surfactant. Absorption-delaying agents such as mono-stearate and gelatin can be contained in the composition and thereby achieve the sustained absorption of the injectable composition.

A single dose for administration is not particularly limited and can be selected appropriately according to the purpose. The single dose is usually about 10 ng/kg to about 250 mg/kg, more preferably about 10 ng/kg to about 1 μg/kg, or about 100 ng/kg to about 100 μg/kg, or about 1 μg/kg to about 1 mg/kg, or about 10 ng/kg to about 50 mg/kg, or about 1 mg/kg to about 100 mg/kg, or about 10 mg/kg to about 50 mg/kg, particularly preferably approximately about 5 mg/kg to about 10 mg/kg. In some embodiments, the single dose is about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 6 mg/kg, about 7 mg/kg, about 8 mg/kg, about 9 mg/kg, about 10 mg/kg, or about 20 mg/kg. As used herein, the term “about” when referring to a measurable value such as a drug dose, is meant to encompass variations of ±20% or ±10%, more preferably ±5%, even more preferably ±1%, and still more preferably ±0.1% from the specified amount, as such variations are appropriate to the disclosed compositions. The dose can be adjusted for each administration according to a symptom to be treated. Alternatively, a dose that falls outside this range may be applied in consideration of the symptom, general status, route of administration, etc. of a patient.

The administration schedule of the pharmaceutical compositions may be any of single-dose administration and continuous administration.

The pharmaceutical compositions of the present invention may be used in combination with one or more additional pharmaceutical medications. The pharmaceutical medications to be combined therewith are appropriately selected in consideration of symptoms or adverse reaction. In the present invention, such combined use also includes the administration of the pharmaceutical medications of the present invention simultaneously or almost simultaneously with the additional pharmaceutical medications as well as the formulation of the pharmaceutical medication of the present invention together with the additional pharmaceutical medications.

The pharmaceutical medications that can be combined with the pharmaceutical composition of the present invention are appropriately selected according to symptoms. Examples of medications include, but are not limited to, anti-thrombotic agents, anti-inflammatory agents, and/or histamine antagonist.

The dosage form, administration route, dose, and administration schedule of the pharmaceutical medication used as a pharmaceutical drug or a pharmaceutical composition for prevention are the same as in use for treatment.

The data obtained from in vitro assays and animal studies, for example, can be used in formulating a range of dosage for use in humans. The dosage of such compositions lies preferably within a range of circulating concentrations that include the ED₅₀ with low, little, or no toxicity. The dosage may vary within this range depending upon the dosage form employed and the route of administration utilized. The therapeutically effective dose of the pharmaceutical compositions can be estimated initially from in vitro assays. A dose may be formulated in mouse models to achieve a circulating plasma concentration range that includes that required to achieve a half-maximal inhibition of symptoms. Protein levels in plasma may be measured, for example, by ELISA, immuno-blot, mass spectrometry, etc. The effects of any particular dosage can be monitored by a suitable bioassay of endpoints.

Unless otherwise indicated, the pharmaceutical compositions of the present invention may be administered at a dose of approximately from about 1.0 ng/kg to about 500 mg/kg, depending on the severity of the symptoms and the progression of the renal pathology. As non-limiting examples, the pharmaceutical compositions may be administered by slow intravenous infusion in an outpatient setting every, e.g., 1, 2, 3, 4, 5, or more days, or by, e.g., weekly, biweekly, monthly, or bimonthly administration. The appropriate therapeutically effective dose of a compound may range approximately from about 1 ng/kg to about 100 mg/kg, from about 1 ng/kg to about 50 mg/kg, from about 1 ng/kg to about 10 mg/kg, from about 1 μg/kg to about 1 mg/kg, from about 10 μg/kg to about 1 mg/kg, from about 10 μg/kg to about 100 μg/kg, from about 100 μg to about 1 mg/kg, and from about 500 μg/kg to about 5 mg/kg. In some embodiments, the appropriate therapeutic dose is chosen from, e.g., about 0.1 mg/kg, about 0.25 mg/kg, about 0.5 mg/kg, about 0.75 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 30 mg/kg, about 40 mg/kg, about 50 mg/kg, about 60 mg/kg, about 70 mg/kg, and about 100 mg/kg.

In some embodiments, the pharmaceutical compositions of the present invention may be administered by intravenous injection at a dose of, e.g., 1.0 mg/kg body weight every two weeks or four weeks at an infusion rate of, e.g., less than or equal to 10, 13, 15, 16, 17, 18, 19, 20, 21, 22, 23, 24, 25, 26, 27, 28, 29, 30, 31, 32, or 33 mg/hour. In another example, the pharmaceutical composition comprising collagen IV protein may be administered by intravenous injection at a dose of, e.g., 20 mg/kg or 40 mg/kg every two or four weeks, over approximately, e.g., 1, 2, 3, 4, 5, 6, 7, 8, 9, or 10 hours.

### Methods for Treating Alport Syndrome

In some embodiments, the present invention provides methods for treating a disease condition characterized by one or more deficiencies of collagen IV protein in a subject in need thereof by administering to the subject a pharmaceutical composition that contains an pharmaceutically effective amount of recombinant collagen IV protein. The condition may be associated with any deficiencies in any one of collagen IV α chain polypeptides selected from α1, α2, α3, α4, α5, and α6 chains. Preferably, the deficiencies are related to collagen IV α3, α4 and α5 chains.

In some aspects, the condition characterized by deficiencies of collagen IV protein is selected from Alport syndrome, thin basement membrane nephropathy (TBMN), familial hematuria, end stage renal disease (ESRD), progressive renal insufficiency, glomerular hematuria, proteinuria, hereditary nephritis, diabetic nephropathy, perinatal cerebral hemorrhage and porencephaly, hemorrhagic stroke, and any diseases or disorder with defects in collagen IV protein, and/or any diseases or disorder with defects in collagen IV protein

In a preferred embodiment, the disease is Alport syndrome. Alport syndrome may be X-linked Alport syndrome, autosomal recessive Alport syndrome, or autosomal dominant Alport syndrome. An X-linked Alport syndrome may be caused by any mutation in COL4A5 gene encoding the α5(IV) chain polypeptide. An autosomal recessive Alport syndrome may be caused by any mutations in COL4A3 and/or COL4A4 genes encoding the α4(IV) chain polypeptide and α5(IV) chain polypeptide. An autosomal dominant Alport syndrome may be caused by any mutations in COL4A3 and/or COL4A4 genes encoding the α4(IV) chain polypeptide and α5(IV) chain polypeptide.

In one embodiment, the subject with Alport syndrome is diagnosed with Alport syndrome with heavy proteinuria, Alport syndrome with mild proteinuria, Alport syndrome with hematuria only, Alport syndrome without renal dysfunction findings who are diagnosed by family history and genetic screening, X-linked syndrome, autosomal recessive Alport syndrome, or autosomal dominant Alport syndrome.

In another embodiment, the condition characterized by one or more deficiencies in COL4A3, COL4A4 and COL4A5 genes further include auditory dysfunction, ocular dysfunction, brain small vessel disease with hemorrhage, brain small vessel disease with Axenfeld-Rieger anomaly or intracerebral hemorrhage.

In some embodiments, the pharmaceutical compositions used in the present methods comprise recombinant collagen IV protomers, dimers, tetramers, multimers and/or a mixture thereof. In some aspects, compositions comprise recombinant collagen IV protomers, wherein protomers are heterotrimers comprising three α(IV) chains selected from the group consisting of the α3(IV), α4(IV) and α5(IV) chains, wherein the three chains form a triple helix. In a preferred embodiment, compositions comprise recombinant collagen IV heterotrimers with one α3(IV) chain, one α4(IV) chain and one α5(IV) chain, wherein the α3(IV) chain comprises the amino acid sequence of SEQ ID NO.3 and variants thereof; the α4(IV) chain comprises the amino acid sequence of SEQ ID NO.4 and variants thereof, and the α5(IV) chain comprises the amino acid sequence of SEQ ID NO.5 and variants thereof.

In other embodiments, recombinant collagen IV protomers may be heterotrimers comprising one, two or three chimeric α chains selected from the chimeric α3(IV), α4(IV), α5(IV) chains, wherein the chimeric α3(IV) chain comprises a chimeric polypeptide in which all or part of the NC1 domain of the α3(IV) chain is replaced with all or part of the NC1 domain of the α1(IV) or α2(IV) chains; the chimeric α4(IV) chain comprises a chimeric polypeptide in which all or part of the NC1 domain of the α4(IV) chain is replaced with all or part of the NC1 domain of the α1(IV) or α2(IV) chains; and the chimeric α5(IV) chain comprises a chimeric polypeptide in which all or part of the NC1 domain of the α5(IV) chain is replaced with all or part of the NC1 domain of the α1(IV) or α2(IV) chains.

In some cases, compositions comprise recombinant collagen IV dimers, wherein said dimers comprise two collagen IV protomers which may be recombinant collagen IV α3-α4-α5 and/or chimeric collagen IV as disclosed herein. In some aspects, collagen IV dimers are dimerized enzymatically or chemically in vitro prior to administering to the subject in need.

In some embodiments, the pharmaceutical composition comprising collagen IV protein is administered to a subject in need thereof by an intravenous injection, intraperitoneal injection, intramuscular injection, subcutaneous injection, intrathecal injection, intracerebral ventricular administration, intracranial delivery, intraocular delivery, intraaural delivery, and/or by an acute or chronically placed catheter. In a preferred embodiment, the recombinant collagen IV protein is administered to a subject in need thereof by intravenous injection.

In some embodiments, the pharmaceutical composition comprising collagen IV protein may be co-administered to a subject in need with one or more prophylactic agents to void thrombosis and inflammatory, and/or other anaphylactic reactions induced by the administration of recombinant collage IV protein to the subject. Such prophylactic agents may include anti-thrombotic agents and/or anti-inflammatories. Anti-thrombotic agents are drugs that reduce thrombus formation. As described herein, anti-thrombotic agents may be used to primarily prevent, or secondarily prevent acute thrombus formation induced by collagen IV replacement. An anti-thrombotic agent may be an antiplatelet drug which limits the aggregation of platelets, an anticoagulant that limits the ability of the blood to clot, or a thrombolytic drug that acts to dissolve clots after they have formed. Antiplatelet drugs may include, but are not limited to, irreversible cyclooxygenase inhibitors such as aspirin and triflusal; adenosine diphosphate (ADP) receptor inhibitors such as clopidogrel, prasugrel, ticagrelor and ticlopidine; phosphodiesterase inhibitors such as cilostazol; glycoprotein IIB/IIIA inhibitors such as abciximab, eptifibatide and tirofiban; adenosine reuptake inhibitors such as dipyridamole; thromboxane inhibitors such as thromboxane synthase inhibitors, thromboxane receptor antagonists and teruthroban. Anticoagulants may include, but are not limited to, warfarin, heparin, acenocoumarol, atromentin, brodifacoum and phenindione. Thrombolytic drugs may include, but are not limited to, tissue plasminogen activator t-PA such as alteplase, reteplase and tenecteplase; anistreplase; streptokinase and urokinase.

In some embodiments, the pharmaceutical composition comprising collagen IV protein may be co-administered to a subject in need with one or more anti-inflammatory agents. Anti-inflammatory agents may include, but are not limited to, NSAIDS (non-steroidal anti-inflammatory drugs) such as aspirin, ibuprofen, naproxen; acetaminophen; ImSAIDs (immune-selective anti-inflammatory drugs); phosphorylated dendrimers (see, e.g., U.S. Patent application publication No. 20100173871). Many other NSAIDS are disclosed in U.S. Pat. Nos. 5,385,941; 5,373,022; 6,730,696; 7,173,018; 7,417,035; 7,741,359; 8,314,140; and 8,541,398; the content of each of which is herein incorporated by reference in their entirety.

In addition to medical drugs, some health/food supplements which are anti-inflammatory may also be used together with the pharmaceutical composition of the present invention, for example, food that create anti-inflammatory prostaglandins (PGE1 and PGE3). Herbs and health supplements having anti-inflammatory qualities may include ginger, turmeric, arnica montana, willow bark, green tea, pineapple bromelain and indian olibanum.

In some embodiments, the anti-thrombotic agents and/or anti-inflammatories may be administered to the subject in need concomitantly, substantially concomitantly, or sequentially, substantially sequentially with the recombinant human collagen IV protein of the present invention.

It is known in the art that protein based medicines often induce innate immune response when administering to a subject. In some embodiments, other agents that can reduce the immune response may be used together the present pharmaceutical compositions comprising collagen IV protein. As non-limiting examples, such drugs may be steroids (e.g. corticosteroids); anti-histamines; antibodies to the complement cascade; and/or those discussed in e.g., U.S. Pat. Nos. 3,167,475; 4,829,077; and 4,902,688.

In some embodiments, the method for treating collagen IV deficiencies further comprise a step of administering to the subject in need one or more agents that promote intravenous extravasation, said agents including hyaluronidase and histamine agonist.

Recent studies have shown that bromine is ubiquitously present in animals as ionic bromide (Br⁻) and is a required cofactor for peroxidasin-catalyzed formation of sulfilimine crosslinks, a posttranslational modification essential for tissue development and architecture found within the collagen IV scaffold of basement membranes (BMs). Bromide, converted to hypobromous acid, forms a bromosulfonium-ion intermediate that energetically selects for sulfilimine formation within collagen IV, an event critical for BM assembly and tissue development (McCall et al., Cell, 2014, 157(6), 1380-1392). Bromine is an essential trace element for animals and bromine dietary supplement can facilitate collagen IV network formation in the GBM.

In accordance with some embodiments of the present invention, one or more cofactors of peroxidasin may be administered to the subject after or substantially after the administration of the recombinant human collagen IV protomers. For example, the patient may have a special diet containing bromide.

In some embodiments, the present invention features methods for preventing, ameliorating one or more abnormalities comprising thinning and splitting glomerular basement membrane (GBM), heavy proteinuria, mild proteinuria, hematuria, renal deficiency, progression to end stage renal disease, auditory dysfunction, ocular abnormalities, porencephaly, brain small vessel disease with hemorrhage, brain small vessel disease with Axenfeld-Rieger anomaly, hereditary angiopathy with nephropathy, aneurysms, and muscle, and/or intracerebral hemorrhage, by administering to a subject in need thereof a pharmaceutical composition that comprises collagen IV protein, such that administering collagen IV protein prevents and/or ameliorates the phenotypic outcomes of the subject.

The collagen IV protein may be administered to a mammal. The mammal may be a mouse, a rat, a dog or a human.

In some further embodiments, the host cells that express chimeric α(IV) polypeptides and/or chimeric cDNA constructs that encode chimeric α(IV) polypeptides may be used in the present methods. Said chimeric α(IV) polypeptides may be selected from chimeric α3(IV), α4(IV), and α5(IV) polypeptides in which all or part of the NC1 domain of each of α3(IV), α4(IV), and α5(IV) polypeptides is replaced with all or part of the NC1 domain of the α1(IV) and/or α2(IV) polypeptides.

### ELISA Assays

ELISA will be used to test the concentration of recombinant collagen IV in the serum or tissues. Collagen IV levels in serum or tissues are altered in many conditions. Serum collagen IV may be indicative of collagen IV degradation in the tissue and may correlate with collagen IV in basement membranes, including GBM. The quantitative measurement of collagen IV may assist in the monitoring of the effectiveness of recombinant collagen IV treatment. An ELISA analysis such as Echelon's collagen IV ELISA Kit may be used for this purpose. According to the manufacturer's proposal, the user simply adds the provided standard curve and their samples to a collagen IV capture plate, following an incubation and plate wash, then adds an HRP labeled detection reagent. After an additional incubation and plate wash, TMB substrate is added to the plate and the colorimetric reaction stopped by the addition of 1N sulfuric acid. The absorbance at 450 nm is measured and the concentration of samples determined by comparison to the standard curve.

### Biomarker Assays

According to the present invention, endogenous molecules present within the blood, tissues and urine may be used to measure the effectiveness of collagen IV replacement. In particular blood and urine samples obtained from the recombinant human collagen IV treated patients are used to test the presence and/or concentrations of biomarkers such as albumin, immunoglobulins A, E, G and M, DBP, RBP, α1 microglubulin, β2 microglubulin, cubulin, apolipoprotein A-1 and megalin.

### Collagen IV Receptor Binding Assay

Integrins are major receptors for extracellular matrix proteins including collagens. Integrin receptors are heterodimers composed of an α and β transmembrane subunit, which are noncovalently bound. Collagen binding is primarily provided by integrins α1β1, α2β1, α10β1 and α11β1. Integrin α10β1 preferentially binds collagen IV, but also binds collagen VI and II.

Cells may also express other collagen receptors such as discoidin domain receptor type 1 (DDR1), discoidin domain receptor type 2 (DDR2), glycoprotein VI (GPVI) and/or mannose receptors. Cells are engineered to present collagen IV receptor integrin (e.g. integrin α10β1) with any techniques well known in the art. Collagen IV proteins at different concentrations are added into the culture media of integrin positive cells, the kinetics of integrin-collagen IV binding, cell migration, adherent morphology of treated cells, and differentiation are analyzed.

### Blood Cell Assays

In some embodiments, blood cells obtained from the subject being treated with recombinant collagen IV may also be used for cell adhesion assays such as focal adhesion kinase (FAK) cell assays. In some embodiments, other cells may be used for cell adhesion assays including human pulmonary fibroblasts. For example, human pulmonary fibroblasts are transfected with vectors expressing a collagen IV integrin receptor and cultured in the collagen IV pre-coated 48 well plates. Cells are cultured in the pre-coated wells for a desired period of time, then unbounded cells are washed away, and the adhered cells are fixed and stained, followed by an extraction step which leads to dye elution from stained cells into supernatant. Thus cell adhesion can be quantified using a colorimetric ELISA plate reader at 595 nm.

Monoclonal antibodies (mAbs) against collagen IV may be used to detect collagen IV protein. Such as mAbs may include those disclosed in U.S. Pat. No. 5,741,652. A collagen IV immunoreactive peptide disclosed in U.S. Pat. No. 8,420,331 may also be used to detect collagen IV.

### Signaling Pathway Assays

Blood cells may be obtained from the subject being treated with recombinant collagen IV protein. The intracellular signaling cascades that relates to collagen IV interaction, and gene expression induced by collagen IV protein may be used to test collagen IV incorporation in the basement membrane.

### Protein Interaction in Cell Free System

The ability of collagen IV to bind other basement membrane components such as laminin-111, collagen VI and biglycan are tested in in vitro binding assays.

Such assays could include ELISA based methods in which laminin-111, collagen VI and biglycan are coated onto a plate, followed by incubation of recombinant collagen IV, followed by detection of collagen IV using an anti-collagen IV antibody chemically conjugated to HRP or other reporter molecule. Other assays such BiaCore could measure the affinities of laminin-111, collagen VI and biglycan to recombinant collagen IV.

### Example

### Example 1: Administration of Collagen IV Protein to Collagen IV Deficiency Animal Models

### Animal Model (COL4A3/COL4A4 Knock Out Model

Cosgrove et al., produced a mouse model for the autosomal form of Alport syndrome by a COL4A3 knockout (Cosgrove et al., Genes Dev., 1996, 10, 2981-2992). The mice developed progressive glomerulonephritis with microhematuria and proteinuria. End-stage renal disease developed at about 14 weeks of age. Transmission electron microscopy (TEM) of glomerular basement membranes (GBM) during development of renal pathology revealed focal multilaminated thickening and thinning beginning in the external capillary loops at 4 weeks and spreading throughout the GBM by 8 weeks. By 14 weeks, half of the glomeruli were fibrotic with collapsed capillaries. Immunofluorescence analysis of the GBM showed the absence of type IV collagen α3, α4, and α5 chains and a persistence of α1 and α2 chains, which are normally localized to the mesangial matrix. Northern blot analysis using probes specific for the collagen chains demonstrated the absence of COL4A3 in the knockout, whereas mRNAs for the remaining chains were unchanged. The progression of Alport renal disease was correlated in time and space with the accumulation of fibronectin, heparan sulfate proteoglycan, laminin-1, and entactin in the GBM of the affected animals.

COL4A3-deficient mice had normal expression of podocyte- and slit diaphragm-associated proteins until 4 weeks after birth, despite significant structural defects in the glomerular basement membrane. At week 5, there were alterations within the slit diaphragm, podocyte effacement, and altered expression of nephrin, a slit diaphragm-associated protein. These findings suggest that defects in glomerular basement membrane proteins lead to an insidious plasma protein leak, while breakdown of the slit diaphragms leads to precipitous plasma protein leak (Hamano et al., J. Biol. Chem., 2002, 277, 31154-31162).

Recently, another mouse Alport syndrome model was identified by mutation in COL4A4 and these mice exhibit a rapid increase of urinary albumin at an early age associated with glomerulosclerosis, interstitial nephritis, and tubular atrophy (Korstanje et al., Kidney International, 2014, 85, 1461-1468).

In one experiment, wild type, Collagen IV α3 chain knockout mice (COL4A3−/−) and/or Collagen IV α4 chain knockout mice (COL4A4−/−) are obtained and maintained under standard conditions, and fed standard mouse chow and water ad libitum. Homozygous deletion of COL4A3 gene is confirmed by PCR reaction as described previously (Cosgrove et al., Genes Dev., 1996, 10, 2981-2992). Mice (wild type, COL4A3+/−, COL4A3−/−) are injected intravenously with collagen IV at various concentrations from 1 ng/kg to 100 mg/kg every day, every other day, weekly or biweekly until a urinalysis demonstrates reduced progression of proteinuria, stabilized proteinuria, or reduced proteinuria, or as long as animal lifespan is maintained.

### Animal Model (COL4A5 Model)

Canine X-linked hereditary nephritis is an animal model for human X-linked Alport syndrome characterized by the presence of a premature stop codon in the α5 (IV) chain polypeptide (Zheng et al., Proc. Nat. Acad. Sci., 1994, 91, 3989-3993). The expression of the canine collagen type IV genes in the kidney indicates that, in addition to a significantly reduced level of COL4A5 gene expression (approximately 10% of normal), expression of the COL4A3 and COL4A4 genes was also decreased to 14-23% and 11-17%, respectively. These findings suggested to a mechanism which coordinates the expression of these 3 basement membrane proteins (Thorner et al., J. Biol. Chem., 1996, 271, 13821-13828). Similarly, the canine X-linked Alport syndrome and control animals are purchased and are injected intravenously with collagen IV at various concentrations from 1 ng/kg to 100 mg/kg every day, every other day, weekly or biweekly until a urinalysis demonstrates reduced progression of proteinuria, stabilized proteinuria, or reduced proteinuria, or as long as animal lifespan is maintained.

Mice Phenotypic Measurements after Administering Collagen IV Protein Intravenously

Urinary albumin and creatinine concentration are estimated using colorimetric assay using commercially available assay kits (e.g., Sigma, St. Louis, Mo.). Urine albumin excretion is estimated as the quotient of urine albumin and urine creatinine as previously described (Sugimoto et al., J Clin Lab Anal., 2003, 17(2), 37-43).

### Histological Assessment of Renal Tissues

Kidney tissues are fixed and stained with Hematoxylin-Eosin (H&E). The extent of renal pathology is assessed by morphometry of the glomerular diseases, tubular atrophy and interstitial fibrosis as previously described. Transmission electron microscopy (TEM) and scanning electron microscopy (SEM) are used to examine the structure of glomerular basement membrane. It is anticipated that improvements in proteinuria may not be coincident with normalization of the GBM architecture and morphology; such as decreased splitting or decreased thickening of the GBM, or reestablishment of foot processes of podocytes, yet the amelioration of such morphological phenotypes in Alport syndrome provide a measure of efficacy. It is anticipated that early treatment of Alport syndrome with recombinant collagen IV will result in normalization of GBM architecture.

### Immunohistochemistry (Collagen IV Expression)

Immunofluorescent staining is performed as described previously (Cosgrove et al., Genes Dev., 1996, 10, 2981-2992). Antibodies specific to either α3(IV), α4(IV) or α5(IV) chain are used to stain collagen IV protein in mice administered with collagen IV. Mice are perfused with 2% PBS buffered formalin before organs are harvested. Cryosectioned tissue specimens are stained with primary antibodies against either α3(IV), α4(IV) or α5(IV) chain for 1 h at room temperature and sections are reacted with fluorescent (e.g., FITC, GFP) conjugated secondary antibodies. Recombinant collagen IV proteins presented in the GBM are fluorescently labeled and analyzed.

### Example 2: Administration of (α1)₂/α2(IV) Collagen to Alport Mouse Model

As described herein, in addition to the major collagen isoform α3/4/5(IV) in the GBM, collagen isoform (α1)₂/α2(IV) network exists in the subendothelial region of the GBM and plays an important role in GBM development and function. It is hypothesized that the defect in Alport GBM is because there is not enough isoform (α1)₂/α2(IV) present to provide the needed stability of collagen network. Experiments are designed to test the hypothesis that infusing isoform (α1)₂/α2(IV) intravenously can increase collagen (α1)₂/α2(IV) levels in the GBM and prevent further development and progression of lesions, and will significantly slow kidney disease progression to kidney failure.

Wild type, Collagen IV α3 chain knockout mice (COL4A3−/−) and/or Collagen IV α4 chain knockout mice (COL4A4−/−) are obtained and maintained under standard conditions, and fed standard mouse chow and water ad libitum. Additionally, mice may be either on the 129S1/SvImJ strain background, or on the B6 background, or on the 129S1/B6 hybrid background. Kidney dysfunction progresses rapidly on the 129S1 background (about 10 weeks), slowly on the B6 background (about 8 months) and intermediately on the 129S1/B6 hybrid background (about 4 months).

In one experiment, Col4a3−/− Alport mice on the 129S1/SvImJ strand background are divided into 3 treatment groups of 7 to 10 mice in each group. Each group is treated by intravenous injection with vehicle only, collagen isoform (α1)₂/α2(IV) at low dose and collagen isoform (α1)₂/α2(IV) at high dose, respectively. Treatment begins at 3 to 4 weeks of age and continues weekly until at least 10 weeks of age, or longer if the treatment proves to be effective at slowing kidney disease progression.

Similar to Example 1, the morphology of GBM, collagen incorporation and kidney function are analyzed after administering collagen isoform (α1)₂/α2(IV). A few mice injected with labeled collagen isoform (α1)₂/α2(IV) at either low dose or high dose are sacrificed at various ages to determine whether the label is concentrated in the GBM.

Urine is analyzed every 1 to 2 weeks for protein and creatinine beginning at 4 weeks of age. Animal weights are determined every 7 to 10 days beginning at 6 weeks of age as a general measure of overall health, as weight loss usually precedes kidney failure. Treated mice are sacrificed at various ages (depending on the results of urine and weight analyses) or at the time of renal failure so that kidney histology and glomerular ultrastructure can be investigated and the effects of the treatments on fibrosis and glomerular basement membrane architecture can be determined.

The results of the analysis allow a determination of whether intravenous collagen isoform (α1)₂/α2(IV) treatment is beneficial for slowing progression of kidney disease. Furthermore, the most effective dose will be determined by the experiment.

### Example 3: Characterization of Mouse Col4 (α1₍₂₎α2) Preparation

Collagen type IV proteins (Col4 (α1₍₂₎α2)) were purified and prepared from mouse tissues. To test species and the relative ratio of protomers, dimers, tetramers and aggregates within the Col4 (α1₍₂₎α2) preparation, denaturing and native gel electrophoresis was used and the size of each band was analyzed.

Several commercial antibodies were evaluated for their capability of binding to Col4 (α1₍₂₎α2) proteins, the antibodies including rabbit polyclonal antibodies (Cat. No. sc70246, Santa Cruz, Dallas, Tex., USA), which recognize internal epitope of human Collagen Type IV α2 chain; rabbit polyclonal antibodies (Cat. No. ab6586, Abcam, Cambridge, Mass., USA), which are raised using immunogen of a full length native purified protein corresponding to human collagen IV aa1-1669 from human placenta; and rabbit polyclonal antibodies (Cat. No. ab19808, Abcam, Cambridge, Mass., USA), which are raised using an immunogen of a full length native collagen IV extracted and purified from tumor tissues of mouse EHS.

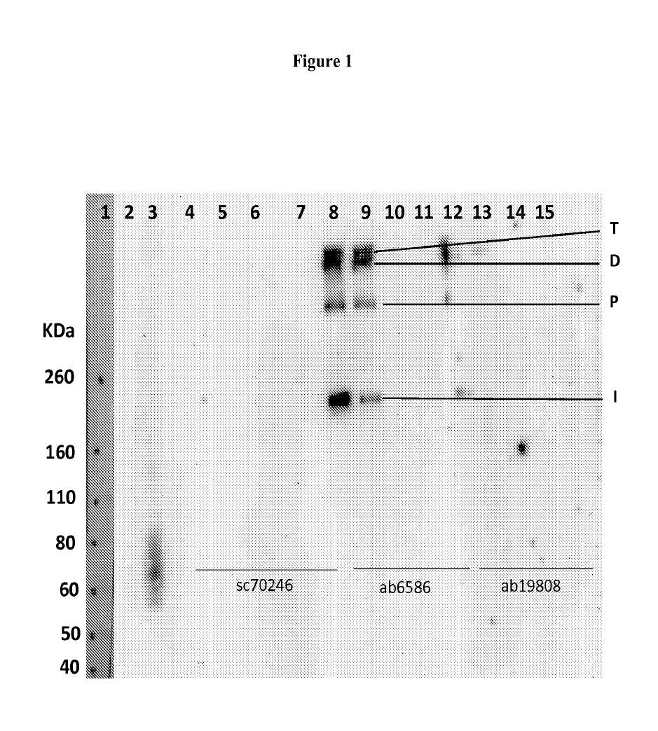

Purified Col4 (α1₍₂₎α2) proteins from mouse (Cat. No. sc-29010, Santa Cruz, Dallas, Tex., USA) were separated using denaturing/non reducing SDS-Polyacrylamide gel electrophoresis (PAGE) and immune blotted with sc-70246 (1:100 dilution), ab6586 (1:1000 dilution) and ab19808 (1:1000 dilution), respectively. HRP conjugated anti-rabbit IgG secondary antibody (1:20,000 dilution) was used to visualize the bands. As shown in FIG. 1, mouse Col4 (α1₍₂₎α2) proteins naturally contain four major species of Col4 (α1₍₂₎α2) including individual α1 and α2 chains (I) (about 180 KDa), protomers (P) (about 480 KDa), dimers (D) (about 900 KDa) and tetramer (T) (larger than 900 KDa). The purification and preparation reserved the full length of polynucleotides and proteins with very little degradation products observed in the Col4 (α1₍₂₎α2) preparation. Among all three anti-Col4 antibodies tested, Antibody ab6586 from Abcam is most sensitive in detecting Col4 (α1₍₂₎α2) proteins.

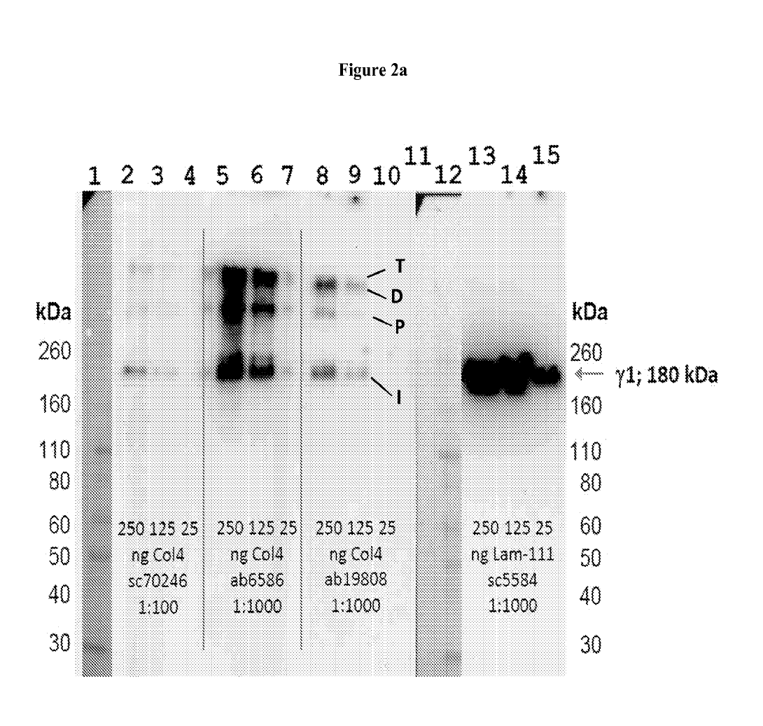

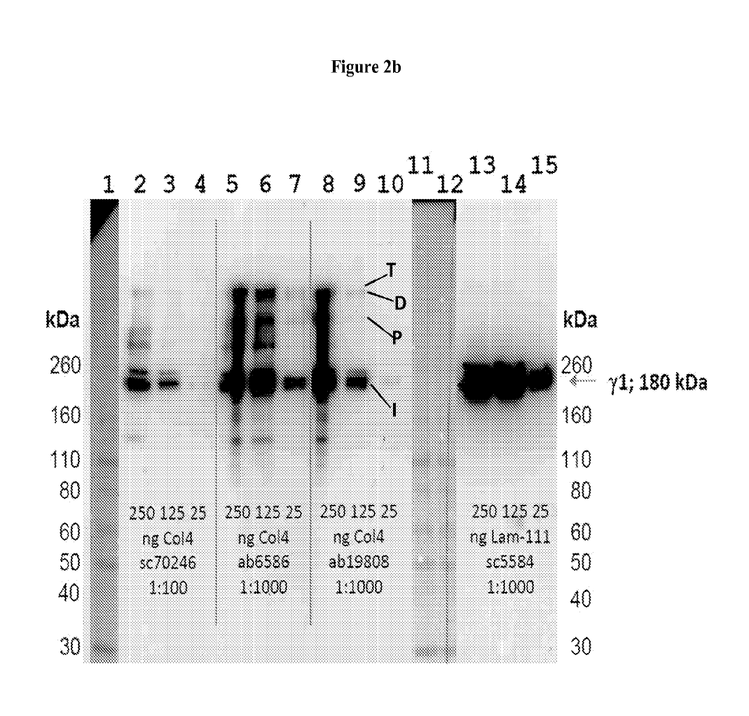

As discussed above, collagen IV proteins are linked and stabilized via the disulfide bonds. A size characterization was further analyzed to test if disulfide reduction resolves Col4 (α1₍₂₎α2) species into individual alpha chains, protomers, dimers and tetramers. Denaturing SDS-PAGEs with or without disulfide reduction were carried and compared. TCEP (Tris (2-carboxyethyl) phosphine) was used to selectively reduce disulfide. As shown in the representative gel images of FIGS. 2A and 2B, Denaturing PAGE without disulfide reduction resolves Col4 (α1₍₂₎α2) species into individual alpha chains (I), protomers (P), dimers (D) and tetramers (T) (FIG. 2A). Denaturing PAGE with disulfide reduction resolves Col4 (α1₍₂₎α2) species mostly into individual alpha chains (I) with some protomers (P), dimers (D) and tetramers (T) (FIG. 2B). This result suggests that native Col4 (α1₍₂₎α2) proteins contain mixtures of disulfide bonded and non-disulfide bonded species, the vast majority of which can be reduced to individual alpha chains. LAM-111 (Cat. No. 23017-015, Life Technologies, Carlsbad, Calif., USA), another structural protein of the GBM, was tested in parallel as an accurate molecular weight standard to compare to the individual alpha chains of Col4 (α1₍₂₎α2).

pH conditions were tested for its effect on the formation of Col4 (α1₍₂₎α2) protomers, dimers and tetramers. Col4 (α1₍₂₎α2) proteins from Santa Cruz (Cat. No. sc-29010) were diluted in acidic solution (50 mM HCl, pH˜2.0), neutral TBS (20 mM Tris-HCl and 500 mM NaCl, pH˜7.5) and basic Tris-HCl (100 mM Tris-HCl, pH˜9.0), respectively, and were analyzed by denaturing SDS-PAGE with or without disulfide reduction. All preparations were assembled for 17 minutes at room temperature before adding the loading sample buffer. The separate bands were visualized by silverstain or immunoblotting using antibody sc6586. No or very little aggregation was observed in all three (acidic, neutral and basic) conditions with or without disulfide reduction. In all three pH conditions, disulfide reduction treatment almost completely reduces high molecular weight dimers (D) and tetramers (T) to protomers (P) and individual alpha chains (I)(data not shown). The results suggest that autocatalytic disulfide formation among individual alpha chains, protomers, dimers and tetramers is a pH dependent process and is reversible.

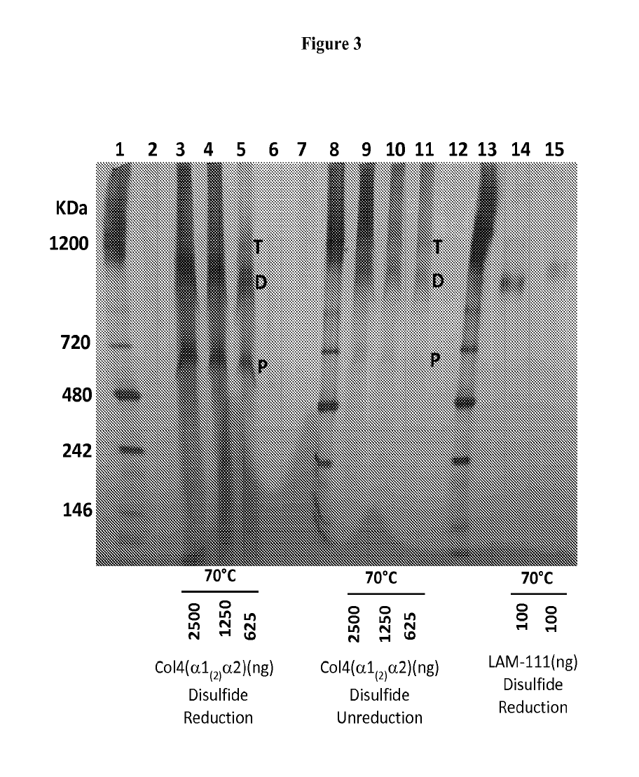

Different charges on alpha polynucleotide chains may affect Collagen IV assembly. We tested if Direct Red 80 charges can shift the ratio of Col4 (α1₍₂₎α2) species. Col4 (α1₍₂₎α2) preparation and LAM-111 were diluted in acidic buffer (50 mM HCl) and loaded in gel sample buffer containing 0.01% Direct Red 80 dye (Cat. No. 365548, Sigma-Aldrich) and analyzed by native PAGE using acidified running buffer containing 0.01% Direct Red 80 dye, with or without disulfide reduction. Native PAGE separation generates a similar Col4 banding to that of denaturing-SDS PAGE. It was demonstrated that Direct Red 80 can charge shift Col 4 and separate the Col4 (α1₍₂₎α2) preparation by native-PAGE. Disulfide reduction of the Col4 (α1₍₂₎α2) preparation at 70° C. can separate protomers (P), dimers (D) and tetramers (T). In unreduced Col4 (α1₍₂₎α2) native preparations, protomers (P), dimers (D) and tetramers (T) are evident, but no aggregations larger than 2MD (FIG. 3).

Altogether, these results indicate that Col4-α1₍₂₎α2 is able to dimerize and tetramerize in vitro.

### Example 4: Platelets Aggregation In Vitro

### Preparation of Resting Platelets

Mouse platelet-rich plasma (PRP) was prepared as described previously (Hoffmeister et al., the clearance mechanism of chilled blood platelets. Cell 2003; 10(1):87-97). All centrifuge steps included prostaglandin E1 to prevent platelet activation. Mouse stain CD-1 was used for the preparation of resting platelets.

Human blood from healthy volunteers, drawn into 0.1 volume of Aster-Jandlanticoagulant, was centrifuged at 100 g for 10 minutes. None of the volunteers had ingested aspirin or other non-steroidal anti-inflammatory drugs for at least 10 days before blood collection. The isolated platelet rich plasma suspension was incubated at 37° C. for up to 1 hour.

### Activation of Resting Platelets

The resting platelets prepared from human blood were incubated with Col4 (α1₍₂₎α2) proteins at different concentrations (Table 4) for 5-10 minutes and activated using 8 uM thrombin receptor-activating peptide (TRAP) (Cat. No. T1573, Sigma-Aldrich, USA).

The resting platelets prepared from mouse were incubated with 4 μl of Col4 (α1₍₂₎α2) protein first and then with additional 40 μl of Col4 (α1₍₂₎α2) protein for 5-10 minutes and activated using 25 uM ADP (Cat. No. 101312, BIO/DATA Corp. USA)

Platelets aggregation begins minutes after activation, and occurs as a result of turning on the GPIIa/b receptor, which allows these receptors to bind the von Willebrand Factor (vWF) or fibrinogen. Activation of platelets change their shapes from curved to straight, and such activation can be detected using Aggregometer (BIO/DATA Corp. Horsham, Pa., USA)

TABLE 4
Platelet aggregation assay

| | | | Col4-α1₍₂₎α2 | |
|---|---|---|---|---|
| | | Total | induced | TRAP induced |
| Col4- | PRP | amount of | Platelet | Platelet |
| α1₍₂₎α2 | (human) | Col4-α1₍₂₎α2 | aggregation | aggregation |
| 4 μl | 400 μl | 5.6 μg/ml | NO | Yes |
| 40 μl | 360 μl | 56 μg/ml | NO | Yes |
| 80 μl | 320 μl | 112 μg/ml | NO | Yes |
| | | | Col4-α1₍₂₎α2 | |
| | | Total | induced | ADP induced |
| Col4- | PRP | amount of | Platelet | Platelet |
| α1₍₂₎α2 | (mouse) | Col4-α1₍₂₎α2 | aggregation | aggregation |
| 4 μl then an | 400 μl | 61.6 μg/ml | NO | Yes |
| additional | | | | |
| 40 μl 1 | | | | |
| minute | | | | |
| later | | | | |

The result shows that Col4 (α1₍₂₎α2) does not activate platelets or induce the aggregations. Furthermore, The Col4 (α1₍₂₎α2) preparation does not inhibit platelet aggregation induced by agonists TRAP or ADP.

### Example 5: In Vitro Labeling of Col4 (α1₍₂₎α2) and LAM-111

To visualize the deposit of injected Col4 (α1₍₂₎α2), in particular the high molecule weigh species of Col4 (α1₍₂₎α2) (about 900 KDa) in the GBM in vivo, Col4 (α1₍₂₎α2) and LAM-111 proteins were first labeled with fluorescein (FITC). In this experiment, 5(6)-SFX (6-(Fluorescein-5-(and-6)-Carboxamido) HexanoicAcid, SuccinimidylEster), mixed isomers (Cat. No. F2181, Molecular Probes), which contains a hexanoic acid spacer, was used for labeling Col4 (α1₍₂₎α2) and LAM-111. 10 mg/ml of 5(6)-SFX was dissolved in 1 ml anhydrous Dimethyl Formamide (10 mg/ml). 2.5 mg Col4 (α1₍₂₎α2) and 1.2 mg LAM-111 was first buffer exchanged to 0.2M Carbonate (pH 8.3) using ZebaSpin Desalting 2 ml Columns (Cat No. 89890, Thermo, USA). 5(6)-SFX solution was then added to 10% (Volume/Volume) and the mixture was stirred at room temperature for 1 hour for the reaction. The mixture was then buffer exchanged to 1×PBS using ZebaSpin Desalting 2 ml Columns.

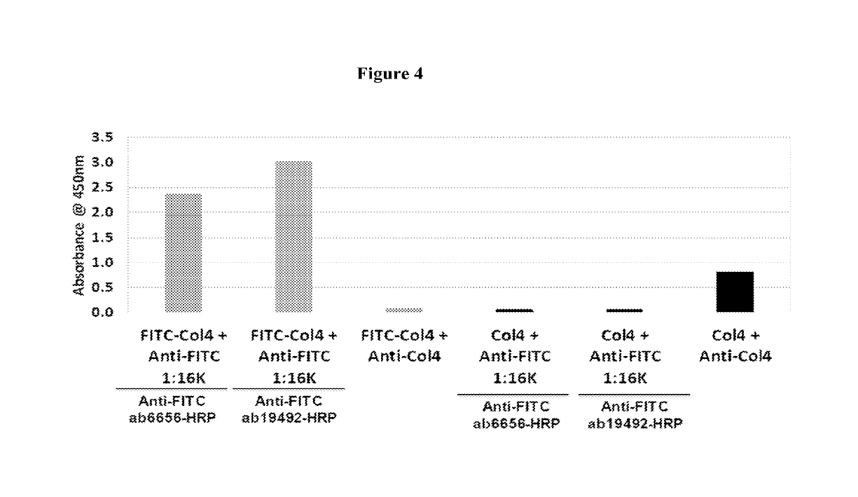

FITC labeled Col4 (α1₍₂₎α2) and FITC-LAM-111 conjugates were tested for the stability using ELISA assay. A rabbit or goat polyclonal anti-FITC-HRP antibody was used to detect FITC-Col4 (α1₍₂₎α2) and FITC-LAM-111 conjugates, whereas a rabbit anti Col4 (α1₍₂₎α2) antibody, together with an anti-rabbit HRP secondary antibody was used to detect both FITC-Col4 (α1₍₂₎α2) conjugate and unlabeled Col4 (α1₍₂₎α2). FIG. 4 illustrates that the tested anti-FITC antibodies ab19492 (rabbit) and ab6656 (goat) from Abcam only detect FITC-Col4 (α1₍₂₎α2) conjugates. The comparison of the staining of anti-FITC and anti-Col4 (α1₍₂₎α2) antibodies indicates that FITC labeled Col4 (α1₍₂₎α2) is diminished, suggesting that extensive FITC labeling may have either masked Col4 (α1₍₂₎α2) epitopes or reduced Col4 (α1₍₂₎α2) stability.

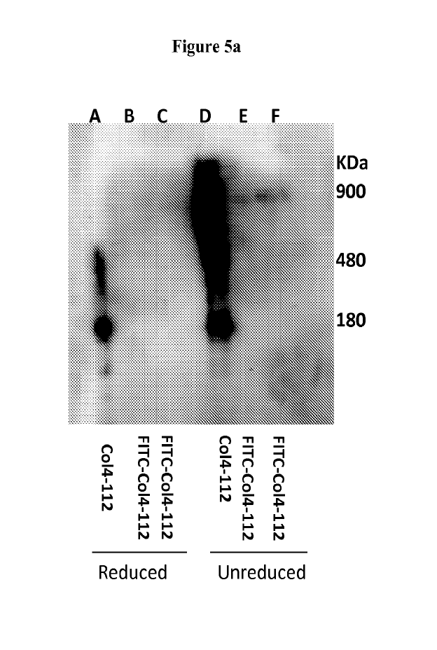

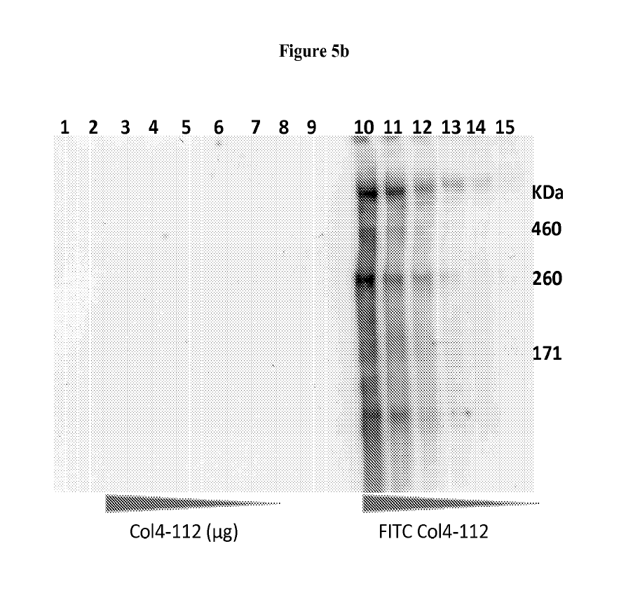

The quality of FITC-Col4 (α1₍₂₎α2) conjugates was analyzed by SDS-PAGE. A representative gel image is shown in FIG. 5a. Consistent with the results of ELISA assays, the band size analysis indicates that detection of FITC-Col4 (α1₍₂₎α2) is greatly diminished, suggesting that extensive FITC labeling may have either masked Col4 epitopes or reduced its stability. However, anti-FITC immunoblot with ab19492 (1:20,000 dilution) revealed sensitive detection of FITC-Col4 (α1₍₂₎α2), which predominantly are dimers and individual chains (shown in FIG. 5b). These results suggest that FITC-Col4 (α1₍₂₎α2) conjugates are suitable for injection if the quantitation of protein concentration and injected amounts are estimated and adjusted.

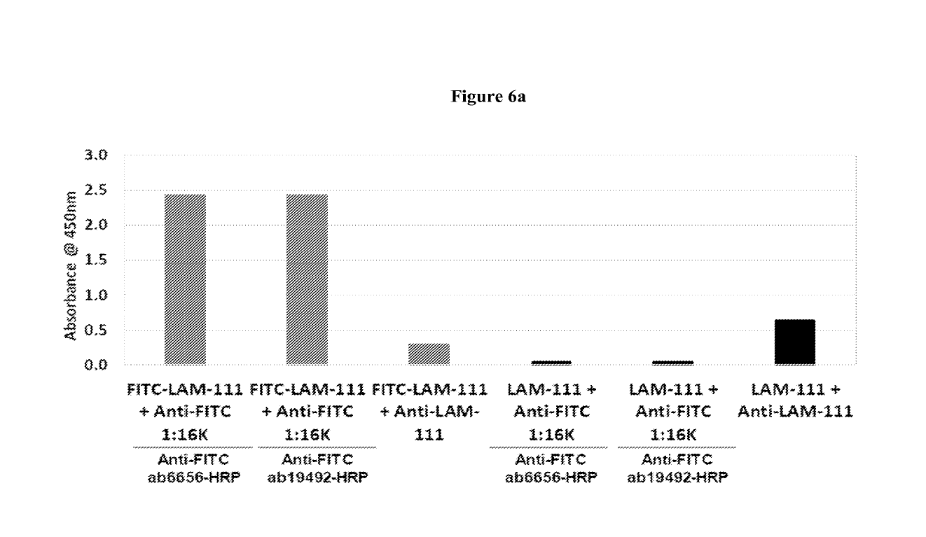

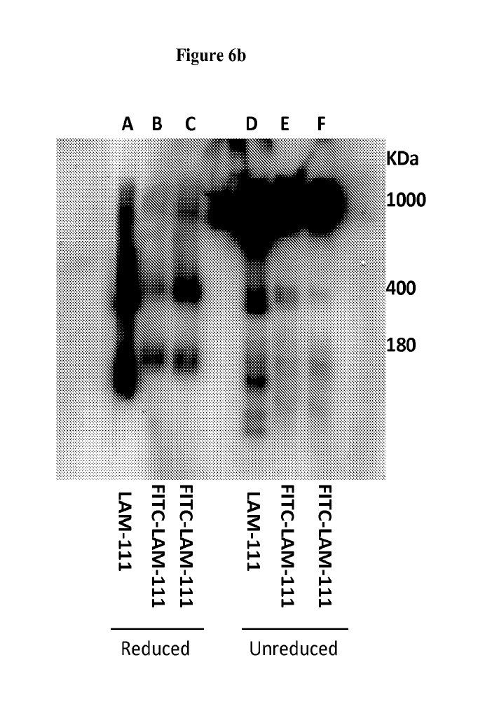

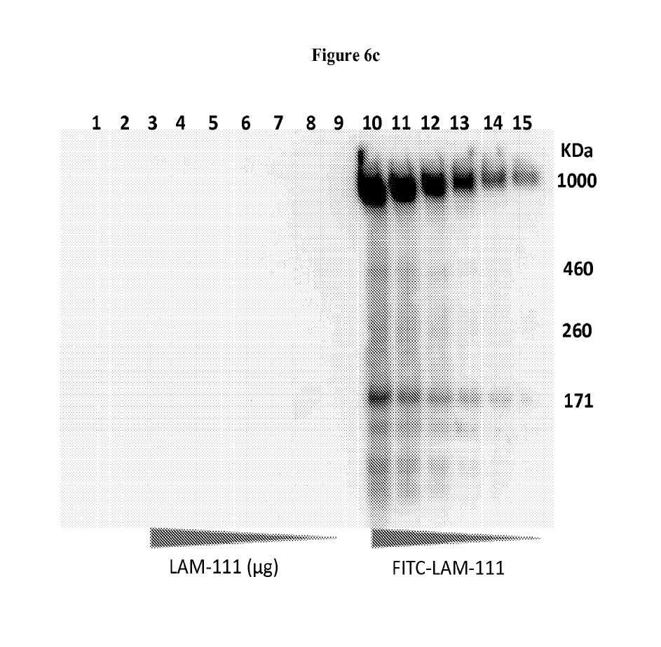

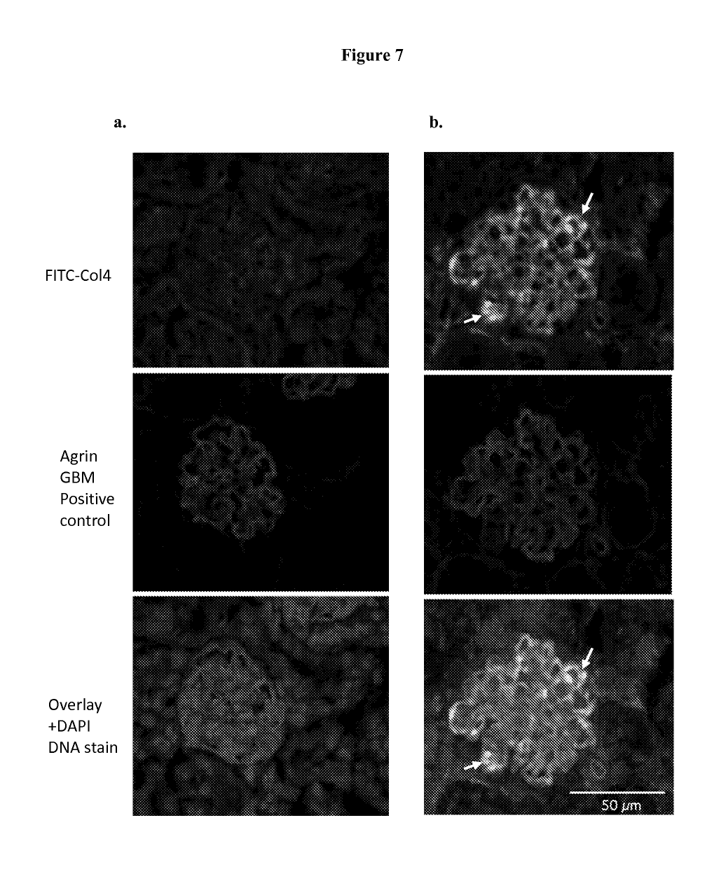

FITC-LAM-111 conjugates are similar to FITC-Col4 (α1₍₂₎α2) conjugates when tested by ELISA assays and immunoblot (FIGS. 6a-6c).

### Example 6: In Vivo Administration of Col4-α1₍₂₎α2 and Detection in the GBM

FITC-Col4 (α1₍₂₎α2) and FITC-LAM-111 conjugates, prepared as described in previous examples, were systemically administrated to the wild type, heterogeneous and Alport mice, and the localization of FITC-Col4 (α1₍₂₎α2) and FITC-LAM-111 conjugates in the GBM of kidney was examined.

Col4+/− and Col4−/− mice at either B6 or 1295, or hybrid background were intravenously injected with either one or 6 doses of FITC-Col4 (α1₍₂₎α2) or FITC-LAM-111 conjugates, respectively. Mice were observed and recorded for any abnormalities and tissue samples were collected at either end of the study or during the intervals of dosing. The dosing schedule and time intervals are listed in Table 5.

Collected tissue samples were processed following standard procedures described in the art for immunofluorescent (IF) staining. Anti-agrin antibody LG1123 (Schlötzer-Schrehardt et al., Exp Eye Res., 2007, 85(6): 845-860) and anti-FITC-HRP antibody ab6656 (Abcam) were used for double staining. Stained samples were examined and staining images were taken and analyzed using confocal microscopy. For each staining, sections of kidney were stained with anti-agrin antibody LG1123 only as a control and FITC signal was examined. No or very weak FITC signals were observed in glomeruli, indicating FITC signals seen in FITC-Col4-α1₍₂₎α2 and FITC-LAM-111 conjugates injected tissue samples are specific to these FITC conjugates. The staining patterns of FITC-Col4-α1₍₂₎α2 protein in glomeruli from each mouse were also summarized in Table 5.

These results demonstrate that systemic administered FITC-Col4-α1₍₂₎α2 and FITC-LAM-111 conjugates (e.g., intravenous injection) can be delivered to kidney and penetrate into the GBM of the mouse kidney. It also suggests that the FITC-Col4-α1₍₂₎α2 deposition into the GBM appears potent.

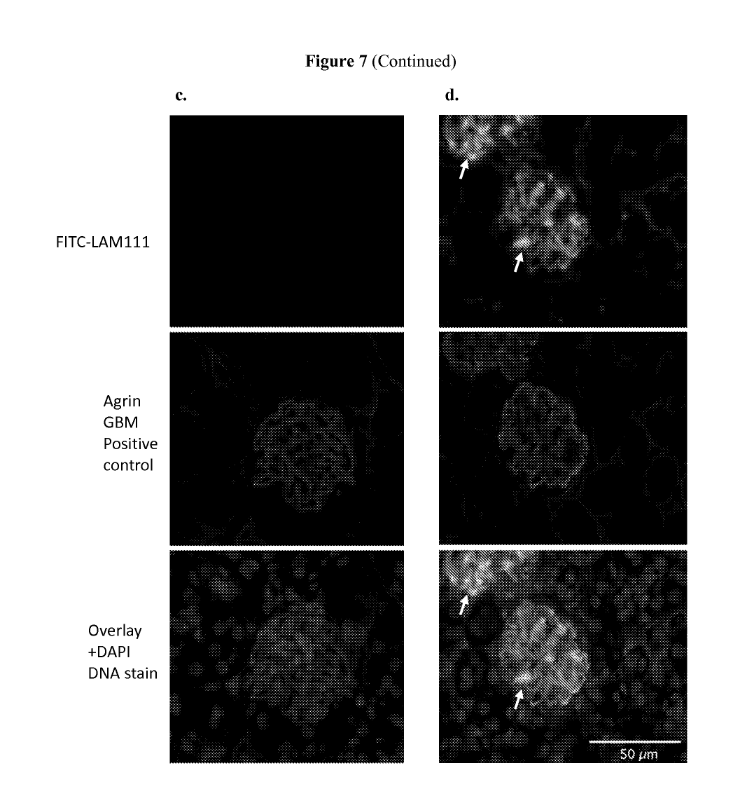

Interestingly, confocal images (FIGS. 7a-7d) show that the FITC-Col4 signals detected are mainly overlapping with Agrin signals but only part of the FITC-LAM-111 signals are overlapping with Agrin signals. That is, the FITC-Col4-α1₍₂₎α2 injected kidney showed more localization of FITC signals to the GBM than the FITC-LAM-111 injected kidney.

No toxicities were observed following up to 6 injections administered over three days. The data demonstrates that the Col4 product, as well as the LAM-111 comparator (each is high molecular weight proteins) are able to deposit into the mouse GBM following systemic delivery.

Such deposition of FITC-Col4-α1₍₂₎α2 and FITC-LAM-111 in the GBM can be further investigated to examine if the deposited Col4-α1₍₂₎α2 proteins can integrate into the collagen network in the GBM and rescue the functionality of Alport GBM. Evaluation of whether chronic repeat dosing of Col4-α1₍₂₎α2 is therapeutic in the Alport mouse model will be studied, such as described in Examples 1 and 2.

In addition, Deposition of FITC-LAM-111 into the GBM indicates that other laminin isoforms, such as LAM-521, may be therapeutic for other kidney diseases such as Pierson Syndrome.

TABLE 5
Systematic administration of FITC labeled Col4-α1₍₂₎α2 and LAM-111

| | | | | | Dual FITC and |
|---|---|---|---|---|---|
| Genotype | | | | | agrin IF |
| and strain | | | | | staining results |
| background | Gender | Age | Injection | Sample collection | in kidney |
| Col4+/− | M | 4.4 m | No injection | Urine collected at | no FITC signals |
| (129S) | | | | 0 hr; | in glomeruli |
| | | | | Urine, kidney, | |
| | | | | lung, liver, | |
| | | | | quadriceps muscle | |
| | | | | collected at 4 hrs | |
| Col4+/−(B6) | M | 3.2 m | No injection | Urine collected at | no FITC signals |
| | | | | 0 hr; | in glomeruli |
| | | | | kidney, lung, | |
| | | | | liver, quadriceps | |
| | | | | muscle collected at | |
| | | | | end of study (at | |
| | | | | 71 hrs) | |
| Col4+/− | F | 2.4 m | No injection | Urine collected at | no FITC signals |
| (Hybrid) | | | | 4 hrs, 28 hrs; | in glomeruli |
| | | | | kidney, lung, | |
| | | | | liver, quadriceps | |
| | | | | muscle collected at | |
| | | | | end of study (at 71 hrs) | |
| Col4−/− (B6) | M | 5.4 m | 1 dose of FITC- | Urine collected at | moderate or |
| | | | LAM-111 | 0 hr; | distinct FITC |
| | | | conjugate at 0 hr | Urine, kidney, | signals in |
| | | | | lung, liver, | glomeruli with |
| | | | | quadriceps muscle | some signals |
| | | | | collected at 4 hrs | seen in agrin- |
| | | | | | positive GBM |
| | | | | | and the rest seen |
| | | | | | in mesangium |
| Col4−/− (B6) | F | 2.9 m | 6 doses of | Urine before each | distinct FITC |
| | | | FITC-LAM-111 | dose and dose | signals in all |
| | | | conjugates at | interval (at 0 hr, | glomeruli with |
| | | | 0 hr, 7 hrs, 22 hrs, | 7 hrs, 22 hrs and | some signals |
| | | | 31 hrs, 46 hrs and | 46 hrs); | seen in agrin- |
| | | | 55 hrs | kidney, lung, liver, | positive GBM |
| | | | | quadriceps muscle | and the rest seen |
| | | | | collected after last | in mesangium |
| | | | | dose (at 71 hrs) | |
| Col4−/− | F | 2.4 m | 6 doses of | Urine collected at | moderate FITC |
| (Hybrid) | | | FITC-Col4- | dose intervals (at | signals in |
| | | | α1(2)α2 | 4 hrs, 28 hrs; | glomeruli with |
| | | | conjugates at | kidney, lung, liver, | most signals |
| | | | 0 hr, 7 hr, 22 hr, | quadriceps muscle | seen in agrin- |
| | | | 31 hrs, 46 hrs and | collected after last | positive GBM; |
| | | | 55 hrs | dose (at 71 hrs) | some clumps |
| | | | | | with bright |
| | | | | | FITC signals |
| | | | | | seen in lumen of |
| | | | | | tubules |
| Col4(−/−) | F | 3 m | 1 dose of FITC- | Urine collected at | moderate FITC |
| (B6) | | | FITC-Col4 | 0 hr and 4 hr after | signals in all |
| | | | conjugate at 0 hr | Col4 dosing; | glomeruli: in |
| | | | | kidney, lung, liver, | some agrin- |
| | | | | quadriceps muscle | positive GBM |
| | | | | collected at 4 hrs | and in |
| | | | | | mesangium |
| Col4(−/−) | F | 2 m | 1 dose of FITC- | Urine collected at | Distinct FITC |
| (B6) | | | FITC-Col4 | 0 hr and 4 hr after | signals in all |
| | | | conjugate at 0 hr | Col4 dosing; | glomeruli: in |
| | | | | kidney, lung, liver, | some agrin- |
| | | | | quadriceps muscle | positive GBM |
| | | | | collected at 4 hrs | and in |
| | | | | | mesangium |
| Col4−/− | F | 4.5 m | 6 doses of | Urine collected | Distinct FITC |
| (B6) | | | FITC-Col4- | before dosing and | signals in all |
| | | | α1(2)α2 | 8 hrs after last dose | glomeruli: in |
| | | | conjugates at | kidney, lung, liver, | agrin positive |
| | | | 0 hr, 7 hr, 24 hr, | quadriceps muscle | GBM and in |
| | | | 31 hrs, 48 hrs and | collected 8 hrs after | mesangium |
| | | | 55 hrs | last dose | |
| Col4−/− | F | 3 m | 6 doses of | Urine collected | Distinct FITC |
| (B6) | | | FITC-Col4- | before dosing and | signals in all |
| | | | α1(2)α2 | 8 hrs after last dose | glomeruli: in |
| | | | conjugates at | kidney, lung, liver, | agrin positive |
| | | | 0 hr, 7 hr, 24 hr, | quadriceps muscle | GBM and in |
| | | | 31 hrs, 48 hrs and | collected 8 hrs after | mesangium |
| | | | 55 hrs | last dose | |
| Col4−/− | F | 2 m | 6 doses of | Urine collected | Distinct FITC |
| (B6) | | | FITC-Col4- | before dosing and | signals in all |
| | | | α1(2)α2 | 8 hrs after last dose | glomeruli: in |
| | | | conjugates at | kidney, lung, liver, | agrin positive |
| | | | 0 hr, 7 hr, 24 hr, | quadriceps muscle | GBM and in |
| | | | 31 hrs, 48 hrs and | collected 8 hrs after | mesangium |
| | | | 55 hrs | last dose | |
| Col4−/− | M | 3 m | 6 injections of | Urine collected | No FITC signals |
| (B6) | | | vehicle at 0 hr, | before injection and | in glomeruli |
| | | | 7 hr, 24 hr, 31 hrs, | 8 hrs after last | |
| | | | 48 hrs and 55 hrs | injection | |
| | | | | kidney, lung, liver, | |
| | | | | quadriceps muscle | |
| | | | | collected 8 hrs after | |
| | | | | last injection | |

### Example 7: Chronic Repeat Dosing of Col4-α1₍₂₎α2 and Therapeutic Effect in Alport Mice

The therapeutic efficacy of collagen IV replacement was tested using Alport mice. Alport and control mice were repeatedly dosed with Col4-α1₍₂₎α2 protein at a dose of 5 mg/kg over a period of time. The injection solution was prepared by mixing 130 μl FITC-Col4-α1₍₂₎α2 (0.5 mg/ml) and 14.5 μl 10×Tris buffered saline. As illustrated in Table 6, mice were dosed twice per week starting at postnatal day 28 (p28) for at least six weeks and the dosing continued if lifespan of a test animal is maintained. Each animal was monitored for their general health and daily lifespan was recorded. Urine samples from each treated animal were regularly collected and further analyzed.

TABLE 6
Chronic repeat dosing efficacy study

| | NO. of | | | | |
|---|---|---|---|---|---|
| | animal | | | Weeks of | Route of |
| Group | (N =) | Genotype | Dose | dosing | administration |
| 1 | 9 | Alport | 5.0 mg/kg | 6+ | Retroorbital |
| | | | | | injection |
| 2 | 6 | Alport | Vehicle | 6+ | Retroorbital |
| | | | | | injection |
| 3 | 5 | Het/WT | vehicle | 6+ | Retroorbital |
| | | | | | injection |

### Results

### Col4-α1(a)α2 Protein Deposit

Similar staining was carried out by dual immunofluorescence immunostaining of Col4-α1₍₂₎α2 and agrin, a known GBM protein in kidney, of mice after repeat dosing of Col4-α1₍₂₎α2 or control vehicle for at least six weeks. Consistent with the previous observations (as discussed in Example 6 and shown in FIGS. 7a-7d), Col4-α1₍₂₎α2 proteins deposit into the glomeruli in kidney and co-localize with other proteins of the GBM (e.g., agrin).

### Morphology of Glomeruli

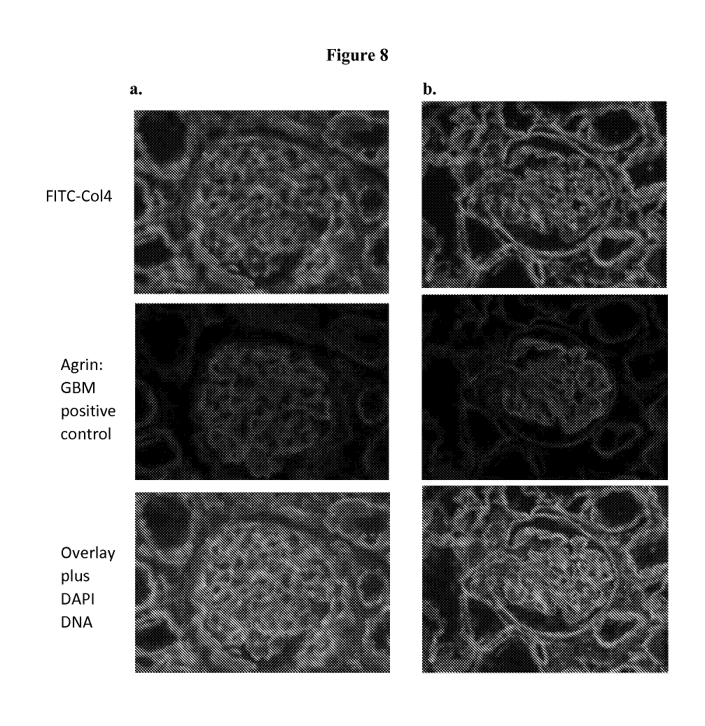

The morphology of glomeruli of experimental mice was also examined. It is indicated that Col4-α1₍₂₎α2 injected Alport mice (Col4−/−) retain open capillary loops and crisp linear staining of the GBM (FIGS. 8a and 8b) and have fewer sclerotic glomeruli and reduced inflammation, as compared to control (i e uninjected or vehicle injected) Alport mice (Col4−/−). As shown in FIG. 8, at least 100 glomeruli from each mouse after 6 weeks of Col4-α1₍₂₎α2 dosing (at postnatal day 70) were counted and analyzed statistically. The statistical data indicates that Col4-α1₍₂₎α2 treated Alport mice (Col4−/−) have 61% of non-sclerotic glomeruli, while un-treated Alport mice (col4−/−) and vehicle treated Alport mice (Col4−/−) have 36% and 29% of non-sclerotic glomeruli, respectively.

### Lifespan

Survival data indicates that Alport mice (Col4−/−) treated with Col4-α1₍₂₎α2 lived longer than vehicle treated Alport mice (Col4−/−). The lifespan of an Alport mouse is the day it must be humanely terminated because its body weight has dropped 15% of its peak weight. Among seven Alport mice treated with Col4-α1₍₂₎α2 two remained alive for 97 and 105 days; well past the ˜90 day lifespan of vehicle treated Alport mice (Table 9). Col4-α1₍₂₎α2, an embryonic isoform of COL4, is already expressed within the adult Alport kidney and yet is known to be more susceptible to proteolytic digestion than Col4-α3/α2/α5 ((Kalluri et al, J. Clin. Invest. 99(10), 1997, 2470-2478; and Gunwar, et al, J. Biol. Chem., 273(15), 1998, 8767-8775). Therefore, the administration and deposition of exogenous Col4-α1₍₂₎α2 into the glomerulus; in conjunction with preexisting Col4-α1₍₂₎α2 appeared to maintain the glomerular basement membrane and delay glomerular sclerosis. Given the resistance of Col4-α3/α2/α5 to proteolytic digestion, treatment of Alport mice with COL4-345 is expected to result in greater efficacy and lifespan than Col4-α1₍₂₎α2, particularly if treatment begins sooner in life. Additionally, no signs of toxicity in treated Alport mice were observed, suggesting that repeat dosing of the highest dose of Col4-α1₍₂₎α2 chronically is safe.

### Glomerular Capillaries

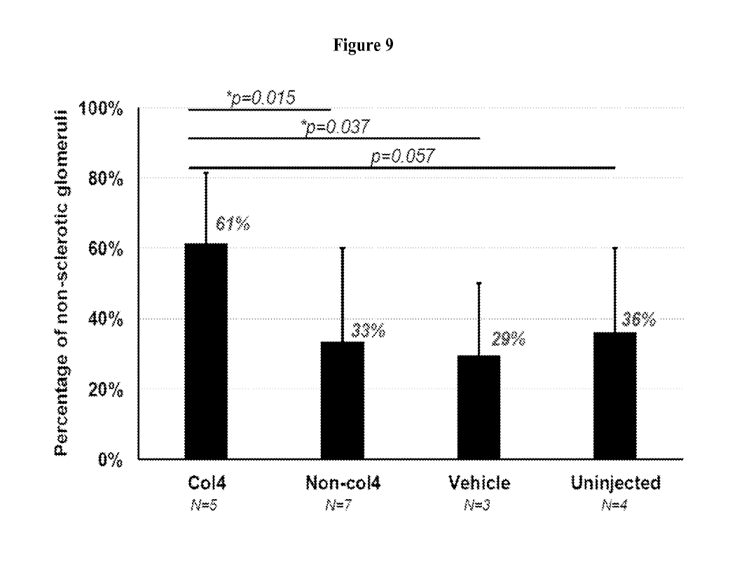

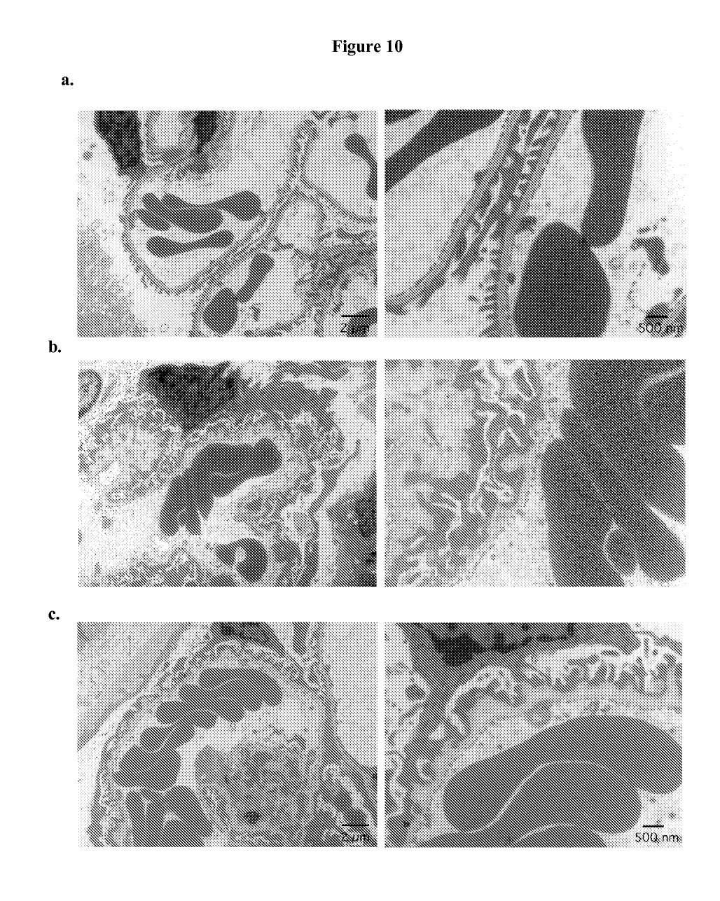

Detailed glomerular capillaries in Alport mice were further analyzed by electron microscopy. The capillary networks in vehicle treated and Col4-α1₍₂₎α2 treated Alport mice (Col4−/−) share similar patterns. The lessons in the glomerular basement membrane don't show significant differences in Col4-α1₍₂₎α2 injected Alport mice (FIG. 9c), as compared to those in vehicle injected Alport mice (FIG. 9b) and both are significantly different from control mice (heterozygous Col4+/− mice) (FIG. 9a).

### Blood Urea Nitrogen (BUN) Analysis

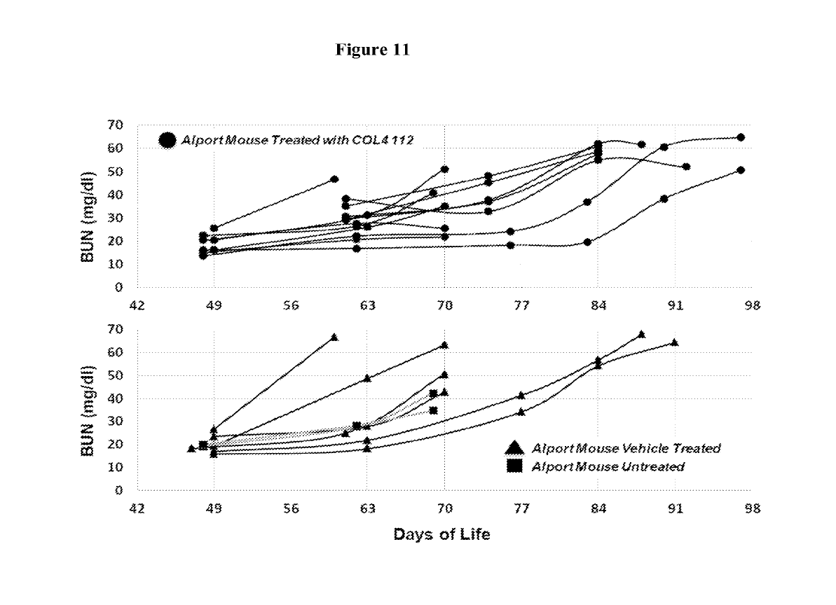

Though no significant difference in glomerular capillaries was observed in Alport mice (Col4−/− injected with Col4−(α1₍₂₎α2), blood urea nitrogen (BUN) test indicates a benefit of exogenous collagen IV proteins in some treated Alport mice, as shown in FIG. 11. Table 7 lists BUN measurements in each treated Alport mouse at different time points during repeat dosing.

TABLE 7
BUN measurements in each treated Alport mouse

| | | ~7 weeks | | ~9 weeks | | ~10 weeks | | ~11 weeks | | ~12 weeks | | ~13 weeks | | ~14 weeks | |
|---|---|---|---|---|---|---|---|---|---|---|---|---|---|---|---|
| Genotype/ | | Age/ | BUN | Age/ | BUN | Age/ | BUN | Age/ | BUN | Age/ | BUN | Age/ | BUN | Age/ | BUN |
| Gender | Treatment | Day | (mg/dl) | Day | (mg/dl) | day | (mg/dl) | Day | (mg/dl) | Day | (mg/dl) | Day | (mg/dl) | Day | (mg/dl) |
| COL−/ | Col4- | | | 61 | 35.2 | | | 74 | 48.1 | 84 | 60.9 | | | | |
| −(129)/(M) | 112 | | | | | | | | | | | | | | |
| COL−/ | Col4- | | | 61 | 29.3 | | | 74 | 45.3 | 84 | 58.9 | | | | |
| −(129)/(M) | 112 | | | | | | | | | | | | | | |
| COL−/ | Col4- | | | 61 | 38.3 | | | 74 | 32.8 | 84 | 55.0 | 92 | 52.1 | | |
| −(129)/(M) | 112 | | | | | | | | | | | | | | |
| COL−/ | Col4- | | | 61 | 30.6 | | | 74 | 37.1 | 84 | 57.7 | 88 | 42.0 | | |
| −(129)/(M) | 112 | | | | | | | | | | | | | | |
| COL−/ | Col4- | | | 61 | 29.5 | | | 74 | 37.8 | 84 | 62.1 | 88 | 61.9 | | |
| −(129)/(F) | 112 | | | | | | | | | | | | | | |
| COL−/ | Col4- | 48 | 20.3 | 62 | 27.7 | 70 | 25.6 | | | | | | | | |
| −(129)/(M) | 112 | | | | | | | | | | | | | | |
| COL−/ | Col4- | 48 | 15.1 | 62 | 20.7 | 70 | 21.8 | | | | | | | | |
| −(129)/(F) | 112 | | | | | | | | | | | | | | |
| COL−/ | Col4- | 48 | 16.2 | 62 | 16.9 | | | 76 | 18.3 | 83 | 19.7 | 90 | 38.3 | 97 | 50.7 |
| −(129)/(F) | 112 | | | | | | | | | | | | | | |
| COL−/ | Col4- | 48 | 13.6 | 62 | 22.2 | | | 76 | 24.3 | 83 | 37.0 | 90 | 60.8 | 97 | 65.1 |
| −(129)/(F) | 112 | | | | | | | | | | | | | | |
| COL−/ | Col4- | 48 | 22.3 | 62 | 26.5 | 69 | 40.8 | | | | | | | | |
| −(129)/(F) | 112 | | | | | | | | | | | | | | |
| COL−/ | Col4- | 49 | 16.1 | 63 | 26.0 | 70 | 35.1 | | | | | | | | |
| −(129)/(M) | 112 | | | | | | | | | | | | | | |
| COL−/ | Col4- | 49 | 20.5 | 63 | 31.4 | 70 | 51.2 | | | | | | | | |
| −(129)/(F) | 112 | | | | | | | | | | | | | | |
| COL−/ | Col4- | 49 | 25.6 | 60 | 46.7 | | | | | | | | | | |
| −(129)/(F) | 112 | | | | | | | | | | | | | | |
| COL−/ | vehicle | 49 | 15.8 | 63 | 18.2 | | | 77 | 34.1 | 84 | 54.1 | 91 | 64.3 | | |
| −(129)/(M) | | | | | | | | | | | | | | | |
| COL−/ | vehicle | 49 | 17.0 | 63 | 21.8 | | | 77 | 41.3 | 84 | 56.7 | 88 | 68.0 | | |
| −(129)/(M) | | | | | | | | | | | | | | | |
| COL−/ | vehicle | 47 | 18.2 | 61 | 24.9 | 70 | 50.5 | | | | | | | | |
| −(129)/(M) | | | | | | | | | | | | | | | |
| COL−/ | vehicle | 49 | 19.3 | 63 | 48.7 | 7 | 63.2 | | | | | | | | |
| −(129)/(F) | | | | | | | | | | | | | | | |
| COL−/ | vehicle | 49 | 26.5 | 60 | 66.8 | | | | | | | | | | |
| −(129)/(F) | | | | | | | | | | | | | | | |
| COL−/ | vehicle | 49 | 23.5 | 63 | 27.9 | 70 | 42.7 | | | | | | | | |
| −(129)/(F) | | | | | | | | | | | | | | | |
| COL−/ | un- | 48 | 19.2 | 62 | 27.7 | 69 | 42.3 | | | | | | | | |
| −(129)/(M) | injected | | | | | | | | | | | | | | |
| COL−/ | un- | 48 | 19.9 | 62 | 28.4 | 69 | 34.8 | | | | | | | | |
| −(129)(M) | injected | | | | | | | | | | | | | | |
| COL+/ | vehicle | | | 61 | 17.2 | | | 74 | 15.8 | 84 | 13.2 | 92 | 30.4 | | |
| +(129)/(F) | | | | | | | | | | | | | | | |
| COL+/(129)/ | vehicle | 48 | 17.8 | 62 | 15.1 | 70 | 20.9 | | | | | | | | |
| (M) | | | | | | | | | | | | | | | |
| COL+/(129)/ | Col4- | 48 | 16.3 | 62 | 20.2 | 70 | 23.0 | | | | | | | | |
| (M) | 112 | | | | | | | | | | | | | | |
| COL+/(129)/ | vehicle | 47 | 16.1 | 61 | 16.8 | 70 | 19.5 | | | | | | | | |
| (M) | | | | | | | | | | | | | | | |
| COL+/ | vehicle | 49 | 17.1 | 63 | 19.1 | 70 | 21.9 | | | | | | | | |
| +(129/(F) | | | | | | | | | | | | | | | |
| COL+/(129)/ | un- | 48 | 21.3 | 62 | 23.0 | 69 | 24.9 | | | | | | | | |
| (M) | injected | | | | | | | | | | | | | | |
| COL+/(129)/ | Col4- | 49 | 17.4 | 63 | 40.8 | 70 | 34.4 | | | | | | | | |
| (M) | 112 | | | | | | | | | | | | | | |

### Urine Albumin to Creatinine Ratio (UACR) Analysis

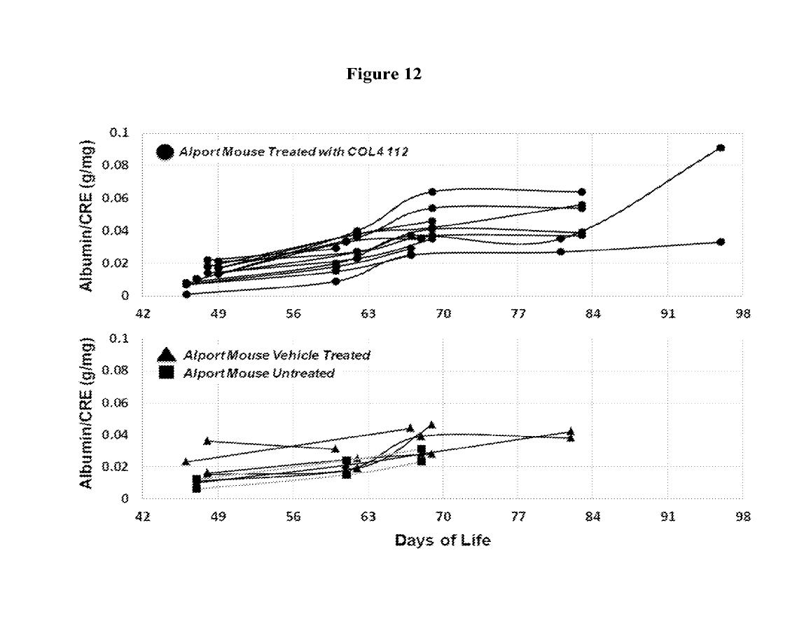

Similarly, urine albumin and creatinine ratios of Alport mice (Col4−/−) injected with Col4−(α1₍₂₎α2), as compared to Alport mice (Col4−/−) injected with vehicle, suggest a benefit of exogenous collagen IV treatment (FIG. 12). Table 8 lists urine Albumin/Creatinine ratios in each treated Alport mouse at different time points during repeat dosing.

TABLE 8
Urine Albumin/Creatinine ratios of each treated Alport mouse

| | | ~7 weeks | | ~9 weeks | | ~10 weeks | | ~12 weeks | | ~14 weeks | |
|---|---|---|---|---|---|---|---|---|---|---|---|
| | | | albumin/ | | albumin/ | | albumin/ | | albumin/ | | albumin/ |
| | | | CRE | | CRE | | CRE | | CRE | | CRE |
| Genotype/Gender | Treatment | Age | (g/mg) | Age | (g/mg) | Age | (g/mg) | Age | (g/mg) | Age | (g/mg) |
| COL−/ | Col4- | 49 d | 0.017 | 62 d | 0.036 | 69 d | 0.054 | 83 d | 0.054 | | |
| −(129)/(M) | 112 | | | | | | | | | | |
| COL−/ | Col4- | 49 d | 0.021 | 62 d | 0.027 | 69 d | 0.037 | 83 d | 0.048 | | |
| −(129) (M) | 112 | | | | | | | | | | |
| COL−/ | Col4- | 49 d | 0.014 | 62 d | 0.027 | 69 d | 0.041 | 83 d | 0.039 | | |
| −(129)/(M) | 112 | | | | | | | | | | |
| COL−/ | Col4- | 49 d | 0.017 | 62 d | 0.038 | 69 d | 0.042 | 83 d | 0.056 | | |
| −(129)/(M) | 112 | | | | | | | | | | |
| COL−/ | Col4- | 49 d | 0.013 | 62 d | 0.040 | 69 d | 0.064 | 83 d | 0.064 | | |
| −(129)/(F) | 112 | | | | | | | | | | |
| COL−/ | Col4- | 46 d | 0.007 | 60 d | 0.018 | 67 d | 0.029 | | | | |
| −(129)/(M) | 112 | | | | | | | | | | |
| COL−/ | Col4- | 46 d | 0.007 | 60 d | 0.015 | 67 d | 0.025 | | | | |
| −(129)/(F) | 112 | | | | | | | | | | |
| COL−/ | Col4- | 46 d | 0.001 | 60 d | 0.009 | 67 d | 0.025 | 81 d | 0.027 | 96 d | 0.033 |
| −(129)/(F) | 112 | | | | | | | | | | |
| COL−/ | Col4- | 46 d | 0.008 | 60 d | 0.020 | 67 d | 0.037 | 81 d | 0.035 | 96 d | 0.091 |
| −(129)/(F) | 112 | | | | | | | | | | |
| COL−/ | Col4- | 47 d | 0.010 | 61 d | 0.033 | 68 d | 0.035 | | | | |
| −(129)/(F) | 112 | | | | | | | | | | |
| COL−/ | Col4- | 48 d | 0.014 | 62 d | 0.023 | 69 d | 0.035 | | | | |
| −(129)/(M) | 112 | | | | | | | | | | |
| COL−/ | Col4- | 48 d | 0.018 | 62 d | 0.038 | 69 d | 0.046 | | | | |
| −(129)/(F) | 112 | | | | | | | | | | |
| COL−/ | Col4- | 48 d | 0.022 | 60 d | 0.029 | | | | | | |
| −(129)/(F) | 112 | | | | | | | | | | |
| COL−/ | vehicle | 47 d | 0.010 | 61 d | 0.021 | 68 d | 0.028 | 82 d | 0.042 | | |
| −(129)/(M) | | | | | | | | | | | |
| COL−/ | vehicle | 47 d | 0.011 | 61 d | 0.018 | 68 d | 0.039 | 82 d | 0.038 | | |
| −(129)/(M) | | | | | | | | | | | |
| COL−/ | vehicle | 46 d | 0.023 | 60 d | | 67 d | 0.044 | | | | |
| −(129)/(M) | | | | | | | | | | | |
| COL−/ | vehicle | 48 d | 0.015 | 62 d | 0.019 | 69 d | 0.046 | | | | |
| −(129)/(F) | | | | | | | | | | | |
| COL−/ | vehicle | 48 d | 0.036 | 60 d | 0.031 | | | | | | |
| −(129)/(F) | | | | | | | | | | | |
| COL−/ | vehicle | 48 d | 0.016 | 62 d | 0.025 | 69 d | 0.028 | | | | |
| −(129)/(F) | | | | | | | | | | | |
| COL−/ | uninjected | 47 d | 0.006 | 61 d | 0.015 | 68 d | 0.023 | | | | |
| −(129)/(M) | | | | | | | | | | | |
| COL−/ | uninjected | 47 d | 0.012 | 61 d | 0.024 | 68 d | 0.031 | | | | |
| −(129)/(M) | | | | | | | | | | | |

As summarized in table 9, exogenous collagen IV protein replacement in Alport (Col4−/−) mice suggests a significant benefit to the syndrome.

TABLE 9
The effects of Col4-(α1₍₂₎α2 replacement in Alport (Col4−/−) mice

| | Alport (Col4−/−) | Alport (Col4−/−) | Alport (Col4−/−) |
|---|---|---|---|
| | un-injected | Vehicle injected | Col 4α112 injected |
| FITC-Col4 IF staining | Negative | Negative | Positive |
| Glomerular | 36% non- | 29% non- | 61% non-sclerotic |
| morphology/pathology | sclerotic | sclerotic | glomeruli |
| (on Day 70) | glomeruli | glomeruli | |
| Lifespan (days; | Not | 88, 91 | 83, 84, 88, 90, 92, 92 |
| individual mice) | Determined | | 97, 105 |
| BUN | NO benefit | NO benefit | Improvements (See |
| | (See detailed | (See detailed | detailed measurements |
| | measurements | measurements in | in Table 7) |
| | in Table 7) | Table 7) | |
| Urine | NO benefit | NO benefit | Improvements (See |
| albumin/Creatinine | (See detailed | (See detailed | detailed measurements |
| ratio | measurements | measurements in | in Table 8) |
| | in Table 8) | Table 8) | |

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

1. A pharmaceutical composition comprising recombinant collagen IV protein and one or more pharmaceutically acceptable excipients.

2. The pharmaceutical composition of claim 1, wherein the recombinant collagen IV protein is a collagen IV protomer, dimer, tetramer, multimer and/or a mixture thereof.

3. (canceled)

4. The pharmaceutical composition of claim 2, wherein said collagen IV protomer is
a) a heterotrimer consisting of three α(IV) polypeptides selected from the group consisting of the α3(IV), α4(IV), and α5(IV) chain polypeptides;
b) a heterotrimer consisting of one α3(IV) chain polypeptide comprising the amino acid sequence of SEQ ID NO.3 and variants thereof one α4(IV) chain polypeptide comprising the amino acid sequence of SEQ ID NO.4 and variants thereof and one α5(IV) chain polypeptide comprising the amino acid sequence of SEQ ID NO.5 and variants thereof
c) a heterotrimer comprising one, two, or three chimeric α3(IV), α4(IV), and α5(IV) chain polypeptides, wherein the chimeric α3(IV) chain polypeptide is a chimeric peptide in which all or part of the NC1 domain of the α3(IV) chain is replaced with all or part of the NC1 domain of α1(IV) or α2(IV) chains; the chimeric α4(IV) chain polypeptide is a chimeric peptide in which all or part of the NC1 domain of the α4(IV) chain is replaced with all or part of the NC1 domain of α1(IV) or α2(IV) chains; and the chimeric α5(IV) chain polypeptide is a chimeric peptide in which all or part of the NC1 domain of the α5(IV) chain is replaced with all or part of the NC1 domain of α1(IV) or α2(IV) chains;
d) a heterotrimer consisting of one chimeric α3(IV) chain polypeptide in which all or part of the NC1 domain of the α3(IV) chain is replaced with all or part of the NC1 domain of α1(IV) or α2(IV) chains; one chimeric α4(IV) chain polypeptide in which all or part of the NC1 domain of the α4(IV) chain is replaced with all or part of the NC1 domain of α1(IV) or α2(IV) chains; and one chimeric α5(IV) chain polypeptide in which all or part of the NC1 domain of the α5(IV) chain is replaced with all or part of the NC1 domain of α1(IV) or α2(IV) chains;
e) a heterotrimer consisting of two copies of the α1(IV) chain polypeptide and one copy of the α2(IV) chain polypeptide; or
f) a heterotrimer consisting of two copies of the α1(IV) chain polypeptide comprising the amino acid sequence of SEQ ID NO.1 and variants thereof; one copy of the α2(IV) chain polypeptide comprising the amino acid sequence of SEQ ID NO.2 and variants thereof.

5-7. (canceled)

8. The pharmaceutical composition of claim 4, wherein the NC1 domains of α1(IV), α2(IV), α3(IV), α4 (IV), α5(IV) comprise the amino acid sequences of SEQ ID NO. 7, SEQ ID NO.8, SEQ ID NO. 9, SEQ ID NO. 10 and SEQ ID NO. 11, respectively.

9. The pharmaceutical composition of claim 2, wherein said recombinant collagen IV protein is a collagen IV dimer, wherein the dimer comprises two protomers of claim 4.

10. The pharmaceutical composition of claim 1, wherein said recombinant collagen IV protein contains 3-hydroxyproline, 4-hydroxyproline and/or hydroxylysine residues, or wherein said one or more pharmaceutically acceptable excipients comprise one or more antioxidants, one or more tonicity agents, and/or one or more chelators.

11-13. (canceled)

14. A pharmaceutical composition for improving glomerular structures and functions in a patient with Alport syndrome comprising recombinant collagen IV protein according to claim 1.

15. A method for treating a condition characterized by one or more deficiencies of collagen IV protein in a subject in need thereof, the method comprising administering to said subject an effective amount of the pharmaceutical composition of claim 1.

16. The method of claim 15, wherein said condition is characterized by one or more deficiencies selected from the group consisting of one or more deficiencies of α3(IV) chain; one or more deficiencies of α4(IV) chain; and one or more deficiencies of α5(IV) chain.

17. The method of claim 16, wherein said one or more deficiencies of α3(IV) chain are caused by mutations in the COL4A3 gene; said one or more deficiencies of α4(IV) chain are caused by mutations in the COL4A4 gene; and said one or more deficiencies of α5(IV) chain are caused by mutations in the COL4A5 gene.

18. The method of claim 17, wherein said condition comprises thin basement membrane nephropathy (TBMN), Alport syndrome, familial hematuria, end stage renal disease (ESRD), progressive renal insufficiency, glomerular hematuria, proteinuria, perinatal cerebral hemorrhage and porencephaly, hemorrhagic stroke, X-linked Alport syndrome, autosomal recessive Alport syndrome, and autosomal dominant Alport syndrome.

19-20. (canceled)

21. The method of claim 18, wherein
a) said subject is a male and said Alport Syndrome is X-linked Alport syndrome;
b) said subject is a male or a female and said Alport syndrome is autosomal recessive Alport syndrome;
c) said subject is a male or a female and said Alport syndrome is autosomal dominant Alport syndrome;
d) Alport syndrome is diagnosed by family history; or
e) the method further comprises co-administering to said subject one or more prophylactic drugs, said prophylactic drugs comprising anti-thrombotics and/or anti-inflammatory drugs.

22-26. (canceled)

27. The method of claim 21, wherein said anti-thrombotic agent is an antiplatelet drug, an anticoagulant, or a thrombolytic drug selected from the group consisting of aspirin, triflusal, clopidogrel, prasugrel, ticagrelor, ticlopidine, cilostazol, abciximab, eptifibatide, tirofiban, dipyridamole, thromboxane synthase inhibitors, thromboxane receptor antagonists, teruthroban, warfarin, heparin, acenocoumarol, atromentin, brodifacoum, phenindione, alteplase, reteplase, tenecteplase, anistreplase, streptokinase and urokinase; or said anti-inflammatory drugs are selected from the group consisting of NSAIDS, acetaminophen, heparin, coumadin, corticosteroids, anti-histamines, and/or antibodies to the complement cascade.

28. (canceled)

29. The method of claim 15, wherein said administration to said subject is delivered by an intravenous injection, intraperitoneal injection, intramuscular injection, subcutaneous injection, intrathecal injection, intracerebral ventricular administration, intracranial delivery, intraocular delivery, intraaural delivery, and/or by an acute or chronically placed catheter.

30-31. (canceled)

32. A method for reversing, ameliorating, slowing, halting, improving or preventing one or more abnormalities in a mammal, the method comprising administering to said mammal the pharmaceutical composition of claim 1.

33. The method of claim 32, wherein said one or more abnormalities comprising thinning and splitting glomerular basement membrane (GBM), heavy proteinuria, mild proteinuria, hematuria, renal deficiency, progression to end stage renal disease, auditory dysfunction, ocular abnormalities, porencephaly, brain small vessel disease with hemorrhage, brain small vessel disease with Axenfeld-Rieger anomaly, hereditary angiopathy with nephropathy, aneurysms, and muscle, and/or intracerebral hemorrhage; or wherein said mammal is a mouse, a rat, a dog or a human.

34. (canceled)

35. A method for producing recombinant collagen IV protein, said method comprising modifying proline residues to generate 3-hydroxyproline and/or 4-hydroxyproline.

36. A cell line for producing recombinant collagen IV protein, wherein said cell line is genetically engineered.

37. (canceled)

38. A chimeric cDNA construct for expressing a chimeric α(IV) chain polypeptide, wherein the chimeric α(IV) chain polypeptide is selected from the group consisting of the chimeric α3(IV), α4(IV) and α5(IV) chain polypeptides.

39. The chimeric cDNA construct of claim 38, wherein the chimeric α3(IV), α4(IV) and α5(IV) chain polypeptides are chimeric peptides in which all or part of the NC1 domain of each of the α3(IV), α4(IV) and α5(IV) chain polypeptides is replaced with all or part of the NC1 domains of α1(IV) and/or α2(IV) chains.

40. The chimeric cDNA construct of claim 39, wherein the NC1 domains of α1(IV), α2(IV), α3(IV), α4(IV), α5(IV) comprise the amino acid sequences of SEQ ID NO.7, SEQ ID NO.8, SEQ ID NO. 9, SEQ ID NO. 10 and SEQ ID NO. 11, respectively.

41. An expression system for producing a chimeric α(IV) polypeptide, wherein the expression system contains the chimeric cDNA of claim 38.

42-50. (canceled)
